(19) United States
(12) Patent Application Publication
(10) Pub. No.: US 2015/0191797 A1
(43) Pub. Date: Jul. 9, 2015

(54) SYSTEMS AND METHODS FOR DIAGNOSING A PREDISPOSITION TO COLON CANCER

(71) Applicant: Institute for Cancer Research d/b/a The Research Institute of Fox Chase Cancer Center, Philadelphia, PA (US)

(72) Inventors: GREG H. ENDERS, VILLANOVA, PA (US); MARK ANDRAKE, HUNTINGTON VALLEY, PA (US); MICHAEL J. HALL, GLENSIDE, PA (US); BIAO LUO, PRINCETON, NJ (US); TIMOTHY J. YEN, HAVERFORD, PA (US)

(21) Appl. No.: 14/660,989

(22) Filed: Mar. 18, 2015

Related U.S. Application Data
(63) Continuation of application No. PCT/US2013/060264, filed on Sep. 18, 2013.
Application No. PCT/US2013/060264 is a continuation-in-part of application No. 13/833,946, filed on Mar. 15, 2013.
(60) Provisional application No. 61/702,423, filed on Sep. 18, 2012.
(60) Provisional application No. 61/731,506, filed on Nov. 30, 2012.

(51) Int. Cl. C12Q 1/68

(52) U.S. Cl. CPC C12Q 1/6886; C12Q 2600/156; C12Q 2600/16; C12Q 2600/158

(57) ABSTRACT

Systems and methods for diagnosing or characterizing a predisposition to colon cancer are provided. Cell nuclei may be evaluated for the presence or quantity of gamma-H2AX foci or their total gamma-H2AX levels. Nucleic acids may be evaluated for the presence, type, or quantity of genomic instability or surrogates of dsDNA breaks such as ataxia telangiectasia mutated (ATM), Rad3-related protein (ATR), and Tumor suppressor p53-binding protein 1 (53BP1) in gamma-H2AX foci. Nucleic acids comprising a germline nucleic acid sequence of the ERCC6, WRN, TERT, SHPRH, and FAAP100 genes may be sequenced or probed to determine if the nucleic acid sequence includes one or more alterations that cause genomic instability, dsDNA breaks, or gamma-H2AX foci or otherwise predispose a subject to develop colon cancer.

### CROSS REFERENCE TO RELATED APPLICATIONS

This application is a continuation of International Application No. PCT/US2013/060264 filed Sep. 18, 2013, which claims priority to U.S. application Ser. No. 13/833,946, filed on Mar. 15, 2013, U.S. Provisional Application No. 61/702,423, filed on Sep. 18, 2012, and U.S. Provisional Application No. 61/731,506, filed on Nov. 30, 2012, the contents of each application are incorporated by reference herein, in their entirety and for all purposes.

### REFERENCE TO A SEQUENCE LISTING

This application includes a Sequence Listing submitted electronically as a text file named Genomic Instability_ST25.txt, created on Sep. 16, 2013 with a size of 250,000 bytes. The Sequence Listing is incorporated by reference herein.

### FIELD OF THE INVENTION

The invention relates generally to the field of cancer diagnostics. More particularly, the invention relates to methods for diagnosing a predisposition to develop colon cancer. The invention also relates to arrays, systems, polynucleotides, and polypeptides, which may be used for practicing diagnostic methods.

### BACKGROUND OF THE INVENTION

Various publications, including patents, published applications, accession numbers, technical articles and scholarly articles are cited throughout the specification. Each of these cited publications is incorporated by reference, in its entirety and for all purposes, in this document.

Colon cancer is the second most common fatal cancer in the United States. About one quarter of colon cancer appears to have an inherited predisposition in that families show a greater frequency of the disease than the general population (e.g., the cancer is familial), and/or the cancer manifests an early age of onset (less than age 50). In most such cases, the molecular cause of the predisposition to cancer is unknown.

Currently, in the absence of such insight, many patients who are suspected of a predisposition to develop colon cancer but do not carry an increased risk needlessly receive frequent invasive and expensive colon examinations, while others who harbor an unrecognized predisposition fail to receive potentially life-saving colon examinations. There is a need for better diagnostics for predicting patient risk factors for developing colon cancer that may aid in early detection, facilitate screening of patients at risk, and reduce the need for invasive tests on patients with reduced risk factors.

### SUMMARY OF THE INVENTION

The invention features methods for diagnosing a predisposition to develop colon cancer. The methods may, for example, comprise determining the quantity of gamma-H2AX foci in a cell or cell nucleus sample obtained from a subject, comparing the determined quantity with reference values for a quantity of gamma-H2AX foci indicative of a predisposition to develop colon cancer, and optionally with reference values for a quantity of gamma-H2AX indicative of a lack of a predisposition to develop colon cancer, and diagnosing whether the subject has a predisposition to develop colon cancer based on the comparison. The methods may comprise determining genomic instability in a nucleic acid sample obtained from a subject, comparing the type of genomic instability with reference values for a type of genomic instability indicative of a predisposition to develop colon cancer, and optionally with reference values for a type of genomic instability indicative of a lack of a predisposition to develop colon cancer, and diagnosing whether the subject has a predisposition to develop colon cancer based on the comparison. The methods may comprise determining double stranded DNA breaks in a nucleic acid sample obtained from a subject, comparing the determined quantity of breaks with reference values for a quantity of breaks indicative of a predisposition to develop colon cancer, and optionally with reference values for a quantity or location of breaks indicative of a lack of a predisposition to develop colon cancer, and diagnosing whether the subject has a predisposition to develop colon cancer based on the comparison. The methods may comprise determining a surrogate of double stranded DNA breaks such as a quantity of one or more of phosphorylated ataxia telangiectasia mutated (ATM), Rad3-related protein (ATR), and Tumor suppressor p53-binding protein 1 (53BP1) in gamma-H2AX foci, comparing the determined quantity of ATM, ATR, and/or 53BP1 in the gamma-H2AX foci with reference values for a quantity of ATM, ATR, and/or 53BP1 in gamma-H2AX foci indicative of a predisposition to develop colon cancer, and optionally with reference values for a quantity of ATM, ATR, and/or 53BP1 in gamma-H2AX foci indicative of a lack of a predisposition to develop colon cancer, and diagnosing whether the subject has a predisposition to develop colon cancer based on the comparison.

The comparing step may be carried out using a processor programmed to compare determined quantities of gamma-H2AX foci with reference values of a quantity of gamma-H2AX foci, or programmed to compare determined types of genomic instability with reference values of types of genomic instability, programmed to compare a determined quantity or location of double stranded DNA breaks with reference values of quantities or locations of double stranded DNA breaks, or programmed to compare a determined quantity of ATM, ATR, and/or 53BP1 in gamma-H2AX foci with reference values for ATM, ATR, and/or 53BP1 in gamma-H2AX foci. The reference values may indicate a high, moderate, low, or no significant probability of a subject having a predisposition to develop colon cancer. The methods may further comprise determining variations in one or more of the ERCC6 gene, the WRN gene, the TERT gene, the SHPRH gene, or the FAAP100 gene associated with causing genomic instability, a DNA damage response, or a predisposition to develop colon cancer. In some aspects, the variations may be any variation described or exemplified herein. Determining such gene variations may be carried out according to any method described or exemplified herein.

In some aspects, the methods comprise determining whether a nucleic acid comprising the ERCC6 gene obtained from a subject encodes a tyrosine at position 180 of the Cockayne Syndrome B protein, and diagnosing whether the subject has a predisposition to develop colon cancer based on the presence or absence of a nucleic acid sequence encoding tyrosine at position 180. In some aspects, the methods comprise determining whether a nucleic acid comprising the WRN gene obtained from a subject encodes an isoleucine at position 705 of the Werner protein, or encodes a tyrosine at position 1292 of the Werner protein, and diagnosing whether the subject has a predisposition to develop colon cancer based on the presence or absence of a nucleic acid sequence encoding isoleucine at position 705 or a nucleic acid sequence encoding tyrosine at position 1292. In some aspects, the methods comprise determining whether a nucleic acid comprising the TERT gene obtained from a subject encodes an arginine at position 198 of the Telomerase Reverse Transcriptase protein, and diagnosing whether the subject has a predisposition to develop colon cancer based on the presence or absence of a nucleic acid sequence encoding arginine at position 198. In some aspects, the methods comprise determining whether a nucleic acid comprising the FAAP100 gene obtained from a subject encodes a leucine at position 466 of the Fanconi anemia associated protein of 100 kD protein, and diagnosing whether the subject has a predisposition to develop colon cancer based on the presence or absence of a nucleic acid sequence encoding leucine at position 466. In some aspects, the methods comprise determining whether a nucleic acid comprising the SHPRH gene obtained from a subject encodes a cysteine at position 1184 of the SNF2 histone linker PHD RING finger helicase protein, and diagnosing whether the subject has a predisposition to develop colon cancer based on the presence or absence of a nucleic acid sequence encoding cysteine at position 1184.

The determining step may comprise determining the sequence of the nucleic acid comprising the ERCC6 gene, comparing the determined sequence with one or more reference nucleic acid sequences encoding a tyrosine at position 180 of the Cockayne Syndrome B protein and optionally one or more reference nucleic acid sequences that do not encode a tyrosine at position 180 of the Cockayne Syndrome B protein, and determining whether the determined sequence encodes a tyrosine at position 180 based on the comparison. The determining step may comprise determining the sequence of the nucleic acid comprising the WRN gene, comparing the determined sequence with one or more reference nucleic acid sequences encoding an isoleucine at position 705 of the Werner protein or one or more reference nucleic acid sequences encoding a tyrosine at position 1292 of the Werner protein, and optionally one or more reference nucleic acid sequences that do not encode an isoleucine at position 705 or a tyrosine at position 1292 of the Werner protein, and determining whether the determined sequence encodes an isoleucine at position 705 or a tyrosine at position 1292 based on the comparison. The determining step may comprise determining the sequence of the nucleic acid comprising the TERT gene, comparing the determined sequence with one or more reference nucleic acid sequences encoding an arginine at position 198 of the Telomerase Reverse Transcriptase protein and optionally one or more reference nucleic acid sequences that do not encode an arginine at position 198 of the Telomerase Reverse Transcriptase protein, and determining whether the determined sequence has the alteration based on the comparison. The determining step may comprise determining the sequence of the nucleic acid comprising the FAAP100 gene, comparing the determined sequence with one or more reference nucleic acid sequences encoding a leucine at position 466 of the Fanconi anemia associated protein of 100 kD and optionally one or more reference nucleic acid sequences that do not encode a leucine at position 466 of the Fanconi anemia associated protein of 100 kD, and determining whether the determined sequence encodes a leucine at position 466 based on the comparison. The determining step may comprise determining the sequence of the nucleic acid comprising the SHPRH gene, comparing the determined sequence with one or more reference nucleic acid sequences encoding a cysteine at position 1184 of the SNF2 histone linker PHD RING finger helicase protein and optionally one or more reference nucleic acid sequences that do not encode a cysteine at position 1184 of the SNF2 histone linker PHD RING finger helicase protein, and determining whether the determined sequence encodes a cysteine at position 1184 based on the comparison. The comparing step may be carried out using a processor programmed to compare determined nucleic acid sequences and reference nucleic acid sequences.

The determining step may comprise contacting the nucleic acid obtained from a subject with one or more polynucleotide probes having a nucleic acid sequence complementary to a nucleic acid sequence encoding a tyrosine at position 180 of the Cockayne Syndrome B protein under stringent conditions, and optionally contacting the nucleic acid obtained from a subject with one or more reference polynucleotide probes having a nucleic acid sequence complementary to a nucleic acid sequence that does not encode a tyrosine at position 180 of the Cockayne Syndrome B protein under stringent conditions, determining whether the one or more probes, and optionally, whether the one or more reference polynucleotide probes, have hybridized with the nucleic acid obtained from the subject, and determining whether the subject has a predisposition to develop colon cancer based on the determination of whether the probes or reference probes have hybridized with the nucleic acid. The determining step may comprise contacting the nucleic acid obtained from a subject with one or more polynucleotide probes having a nucleic acid sequence complementary to a nucleic acid sequence encoding an isoleucine at position 705 of the Werner protein under stringent conditions, or one or more polynucleotide probes having a nucleic acid sequence complementary to a nucleic acid sequence encoding a tyrosine at position 1292 of the Werner protein under stringent conditions, and optionally contacting the nucleic acid obtained from a subject with one or more polynucleotide probes having a nucleic acid sequence complementary to a nucleic acid sequence that does not encode an isoleucine at position 705 or a tyrosine at position 1292 of the Werner protein, determining whether the one or more probes, and optionally, whether the one or more reference probes, have hybridized with the nucleic acid obtained from the subject, and determining whether the subject has a predisposition to develop colon cancer based on the determination of whether the probes have hybridized with the nucleic acid. The determining step may comprise contacting the nucleic acid obtained from a subject with one or more polynucleotide probes having a nucleic acid sequence complementary to a nucleic acid sequence encoding an arginine at position 198 of the Telomerase Reverse Transcriptase protein under stringent conditions, and optionally contacting the nucleic acid obtained from a subject with one or more polynucleotide probes having a nucleic acid sequence complementary to a nucleic acid sequence that does not encode an arginine at position 198 of the Telomerase Reverse Transcriptase protein under stringent conditions, determining whether the one or more probes, and optionally, whether the one or more reference probes, have hybridized with the nucleic acid obtained from the subject, and determining whether the subject has a predisposition to develop colon cancer based on the determination of whether the probes have hybridized with the nucleic acid. The determining step may comprise contacting the nucleic acid obtained from a subject with one or more polynucleotide probes having a nucleic acid sequence complementary to a nucleic acid sequence encoding a leucine at position 466 of the Fanconi anemia associated protein of 100 kD under stringent conditions, and optionally contacting the nucleic acid obtained from a subject with one or more reference polynucleotide probes having a nucleic acid sequence complementary to a nucleic acid sequence that does not encode a leucine at position 466 of the Fanconi anemia associated protein of 100 kD under stringent conditions, determining whether the one or more probes, and optionally, whether the one or more reference polynucleotide probes, have hybridized with the nucleic acid obtained from the subject, and determining whether the subject has a predisposition to develop colon cancer based on the determination of whether the probes or reference probes have hybridized with the nucleic acid. The determining step may comprise contacting the nucleic acid obtained from a subject with one or more polynucleotide probes having a nucleic acid sequence complementary to a nucleic acid sequence encoding a cysteine at position 1184 of the SNF2 histone linker PHD RING finger helicase protein under stringent conditions, and optionally contacting the nucleic acid obtained from a subject with one or more reference polynucleotide probes having a nucleic acid sequence complementary to a nucleic acid sequence that does not encode a cysteine at position 1184 of the SNF2 histone linker PHD RING finger helicase protein under stringent conditions, determining whether the one or more probes, and optionally, whether the one or more reference polynucleotide probes, have hybridized with the nucleic acid obtained from the subject, and determining whether the subject has a predisposition to develop colon cancer based on the determination of whether the probes or reference probes have hybridized with the nucleic acid. The nucleic acid may be comprised within a cell, and the method may comprise contacting the nucleic acid in the cell with the one or more polynucleotide probes, and optionally with the one or more reference polynucleotide probes. If more than one probe was contacted with the nucleic acid, the method may comprise the step of identifying which of the probes hybridized with the nucleic acid.

The methods may further comprise determining the presence or absence of genomic instability in subjects determined to have one or more of the ERCC6, WRN, TERT, SHPRH, or FAAP100 gene alterations described or exemplified herein. Genomic instability may comprise aneuploidy or polyploidy among the subject's chromosomes. Genomic instability may comprise one or more of chromosomal translocations, chromosomal inversions, chromosome deletions, broken DNA chains, or abnormal DNA structure. Genomic instability may comprise double stranded DNA breaks. Determining the presence or absence of genomic instability may be carried out using any methodology suitable in the art, including those described or exemplified herein. Such methods include, without limitation, karyotyping, metaphase spreads, flow cytometry of propidium iodide-stained cells, immunofluorescence, immunohistochemistry, and determination of the activation of a DNA damage response.

A nucleic acid sequence encoding tyrosine at position 180 may comprise an A to T substitution in the codon encoding asparagine at position 180 of the Cockayne Syndrome B protein. The A to T substitution may occur at a position corresponding to position number 50,408,777 in the ERCC6 gene locus of human chromosome number 10. The Cockayne Syndrome B protein may comprise the amino acid sequence of SEQ ID NO:5.

A nucleic acid sequence encoding an isoleucine at position 705 of the Werner protein may comprise a C to T substitution in the codon encoding threonine at position 705 of the Werner protein. The C to T substitution may occur at a position corresponding to position number 31,088,698 in the WRN gene locus of human chromosome number 8. A nucleic acid sequence encoding a tyrosine at position 1292 of the Werner protein may comprises a C to A substitution in the codon encoding serine at position 1292 of the Werner protein. The C to A substitution may occur at a position corresponding to position number 31,134,481 in the WRN gene locus of human chromosome number 8. The Werner protein may comprise the amino acid sequence of SEQ ID NO:10.

A nucleic acid sequence encoding arginine at position 198 may comprise a G to C substitution in the codon encoding glycine at position 198 of the Telomerase Reverse Transcriptase protein. The G to C substitution may occur at a position corresponding to position number 1,347,409 in the TERT gene locus of human chromosome number 5. The Telomerase Reverse Transcriptase protein may comprise the amino acid sequence of SEQ ID NO:19.

A nucleic add sequence encoding leucine at position 466 may comprise a C to T substitution in the codon encoding serine at position 466 of the Fanconi anemia associated protein of 100 kD. The C to T substitution may occur at a position corresponding to position number 77,124,711 in the FAAP100 gene locus of human chromosome number 17. The Fanconi anemia associated protein of 100 kD may comprise the amino acid sequence of SEQ ID NO:24.

A nucleic acid sequence encoding cysteine at position 1184 may comprise a C to T substitution in the codon encoding arginine at position 1184 of the SNF2 histone linker PHD RING finger helicase protein. The C to T substitution may occur at a position corresponding to position number 146,243,968 in the SHPRH gene locus of human chromosome number 6. The SNF2 histone linker PHD RING finger helicase protein may comprise the amino acid sequence of SEQ ID NO:29 or SEQ ID NO:30.

The invention also features isolated polynucleotides. The polynucleotides may be affixed to a support, including an array. In some aspects, an isolated polynucleotide comprises the ERCC6 gene comprising a nucleic acid sequence encoding a tyrosine at position 180 of the Cockayne Syndrome B protein. The ERCC6 gene may comprise an A to T substitution at a position corresponding to position number 50,408,777 in the ERCC6 gene locus of human chromosome number 10. The nucleic acid sequence may comprise SEQ ID NO:1. The nucleic acid sequence may encode the amino acid sequence of SEQ ID NO:4.

In some aspects, an isolated polynucleotide comprises the WRN gene comprising a nucleic acid sequence encoding an isoleucine at position 705 of the Werner protein. The WRN gene may comprises a C to T substitution at a position corresponding to position number 31,088,698 in the WRN gene locus of human chromosome number 8. The nucleic acid sequence may comprise SEQ ID NO:6. The nucleic acid sequence may encode the amino acid sequence of SEQ ID NO:9.

In some aspects, an isolated polynucleotide comprises the WRN gene comprising a nucleic acid sequence encoding a tyrosine at position 1292 of the Werner protein. The WRN gene may comprise a C to A substitution at a position corresponding to position number 31,134,481 in the WRN gene locus of human chromosome number 8. The nucleic acid sequence may comprise SEQ ID NO:13. The nucleic acid sequence may encode the amino acid sequence of SEQ ID NO:14. The nucleic acid sequence may comprise SEQ ID NO:11. The nucleic acid sequence may encode the amino acid sequence of SEQ ID NO:12.

In some aspects, an isolated polynucleotide comprises the TERT gene comprising a nucleic acid sequence encoding an arginine at position 198 of the Telomerase Reverse Transcriptase protein. The TERT gene may comprise a G to C substitution at a position corresponding to position number 1,347,409 in the TERT gene locus of human chromosome number 5. The nucleic acid sequence may comprise SEQ ID NO:15. The nucleic acid sequence may encode the amino acid sequence of SEQ ID NO:18.

In some aspects, an isolated polynucleotide comprises the FAAP100 gene comprising a nucleic acid sequence encoding a leucine at position 466 of the Fanconi anemia associated protein of 100 kD. The FAAP100 gene may comprises a C to T substitution at a position corresponding to position number 77,124,711 in the FAAP100 gene locus of human chromosome number 17. The nucleic acid sequence may comprise SEQ ID NO:20. The nucleic acid sequence may encode the amino acid sequence of SEQ ID NO:23.

In some aspects, an isolated polynucleotide comprises the SHPRH gene comprising a nucleic acid sequence encoding a cysteine at position 1184 of the SNF2 histone linker PHD RING finger helicase protein. The SHPRH gene may comprises a C to T substitution at a position corresponding to position number 146,243,968 in the SHPRH gene locus of human chromosome number 6. The nucleic acid sequence may comprise SEQ ID NO:25. The nucleic acid sequence may encode the amino acid sequence of SEQ ID NO:28.

### BRIEF DESCRIPTION OF THE DRAWINGS

FIG. 1A shows a metaphase spread (right panel) from patient 120713, showing a gain of chromosome 9 as identified on the ordered array (left panel). Phytohemaglutinen (PHA)-stimulated peripheral blood lymphocytes were used as a source of the chromosomes. FIG. 1B shows a second metaphase spread (right panel) from patient 120713, showing a gain of chromosome 11 as identified on the ordered array (left panel). Two other gains were identified among 50 well-separated metaphase spreads in patient 120713 (not shown).

FIG. 2 shows an increased 5 phase fraction in patient 118294 compared to its matched control (102615) and all other samples, and shows an increase G2/M fraction in patient 120713 compared to its matched control (237313) and all other samples. The plots were obtained from flow cytometry analysis of propidium iodide (PO-stained, PHA-stimulated lymphocytes. 5 phase fractions are marked by hatching. G2/M phase fractions are shown in dark grey on the right-hand side of the plot.

FIG. 3 shows multiple reads of the ERCC6 variant from patient 120713, affirming validity of the N180Y change in the CSB protein sequence.

FIG. 4 shows the location of the CSB protein variant N180Y (arrow) in patient 120713 within a highly conserved region predicted to be a surface-exposed region of the protein, and therefore functionally significant. Amino acids predicted to be functionally significant are designated by bold typeface.

FIG. 5 shows multiple reads of the WRN gene sequence variants from patients 120713 and 118294, affirming validity of the T705I (top panel) and S1292Y (bottom panel) changes in the Werner protein sequence.

FIG. 6 shows greater DDR foci in patient 120713 than its control. Lymphocytes were treated with 4 J/m² UV or 3 μM aphidicolin for 2 h and fixed 5 h (UV) or 1 h later (aph). γH2AX IF foci were scored in blinded fashion. Results shown are the percent of cells (y-axis) with ≧10 nuclear foci (total of 1,117 cells scored). Insert: 120713 cell foci after UV. Comparison with (no) Rx: P=0.08.

FIG. 7 shows greater gamma-H2AX foci in lymphocytes from patient 120713 than the control in another experiment. The foci are higher at the baseline (no Rx), as well as in response to amphidicolin, camptothecin, and etopside treatments.

FIG. 8A shows a Coomassie blue stain of recombinant wildtype (WT) and variant (T646I) Werner protein (arrow). FIG. 8B shows the loss of helicase activity in the Werner protein in patient 120713.

FIG. 9 shows reduced nucleosome remodeling in the patient 120713 ERCC6 variant.

FIG. 10 shows increased gamma-H2AX foci in patient 118294 following treatment with aphidicolin, relative to the control (con).

FIG. 11 shows greater gamma-H2AX levels in untreated cells from patient 118294 (Pt) relative to the control (con).

### DETAILED DESCRIPTION OF THE INVENTION

Various terms relating to aspects of the present invention are used throughout the specification and claims. Such terms are to be given their ordinary meaning in the art, unless otherwise indicated. Other specifically defined terms are to be construed in a manner consistent with the definition provided in this document.

As used throughout, the singular forms “a,” “an,” and “the” include plural referents unless expressly stated otherwise.

A molecule such as a polynucleotide has been “isolated” if it has been removed from its natural environment and/or altered by the hand of a human being.

A nucleotide in a nucleic acid sequence such as but not limited to a cDNA, mRNA, or derivative thereof may correspond to a nucleotide in the genomic nucleic acid sequence. In this respect, corresponding to comprises a positional relationship of nucleotides in the genomic DNA gene sequence relative to nucleotides in a polynucleotide sequence (e.g., cDNA, mRNA) obtainable from the genomic DNA sequence.

The terms subject and patient are used interchangeably. A subject may be any animal, and preferably is a mammal. A mammalian subject may be a farm animal (e.g., sheep, horse, cow, pig), a companion animal (e.g., cat, dog), a rodent or laboratory animal (e.g., mouse, rat, rabbit), or a non-human primate (e.g., old world monkey, new world monkey). Human beings are highly preferred.

It has been observed in accordance with the invention that certain variations, which include deletions, substitutions, rearrangements, and combinations thereof, in the germline nucleic acid sequence of one or more of the Excision Repair Cross-Complementing Rodent Repair Deficiency Complementation Group 6 (ERCC6) gene, the Werner Syndrome RecQ Helicase-like (WRN) gene, the Telomerase Reverse Transcriptase (TERT) gene, the Fanconi anemia associated protein of 100 kD (FAAP100) gene, and the SNF2 histone linker PHD RING finger helicase (SHPRH) gene predispose subjects having such variations to genomic instability, double stranded DNA breaks, and/or extensive phosphorylation of the histone H2AX, forming gamma-H2AX foci proximal to the DNA breaks. It has also been observed that certain DNA damage response proteins such as phosphorylated ataxia telangiectasia mutated (ATM), Rad3-related protein (ATR), and Tumor suppressor p53-binding protein 1 (53BP1) are recruited into such foci. Without intending to be limited to any particular theory or mechanism of action, it is believed that such genomic instability, double stranded DNA breaks, and/or enhanced gamma-H2AX foci are markers of a predisposition to develop colon cancer. The quantity of gamma-H2AX foci may be serve, at least in part, as a proxy for underlying variations in certain proteins, particularly those that play a role in DNA repair, which variations impair the biologic activity of such proteins and, ultimately, impair DNA repair. Accordingly, the invention features methods for diagnosing a predisposition to develop colon cancer. Any of the methods may be carried out in vivo, in vitro, or in situ.

In general, the methods comprise determining genomic instability and/or double stranded DNA breaks in a nucleic acid sample obtained from a subject, and/or determining gamma-H2AX foci in a cell or cell nucleus sample obtained from a subject. Determining genomic instability, double stranded DNA breaks, and/or gamma-H2AX foci may be carried out according to any suitable method, including the methods described or exemplified herein. The determined genomic instability, double stranded DNA breaks, and/or gamma-H2AX foci may be compared with quantitative or qualitative reference values for genomic instability, double stranded DNA breaks, and/or gamma-H2AX foci associated with a predisposition to develop colon cancer, and optionally with quantitative or qualitative reference values for genomic instability, double stranded DNA breaks, and/or gamma-H2AX foci not associated with a predisposition to develop colon cancer, for example, reference values of a healthy subject or a subject not at risk to develop colon cancer based on these markers. The reference values may, for example, comprise values indicative of a high risk for developing colon cancer, values indicative of a moderate risk for developing colon cancer, and/or values indicative of a low risk for developing colon cancer. The comparing step may be carried out using a processor programmed to compare determined quantitative or qualitative values for genomic instability, double stranded DNA breaks, and/or gamma-H2AX foci with quantitative or qualitative reference values for such markers.

The methods for diagnosing a predisposition to develop colon cancer may further comprise (e.g., in addition to determining genomic instability, double stranded DNA breaks, and/or gamma-H2AX foci), or comprise in the alternative (e.g., without determining genomic instability, double stranded DNA breaks, and/or gamma-H2AX foci), identifying germline nucleic acid sequence alterations in the ERCC6, WRN, TERT, SHPRH and/or FAAP100 genes that predispose a subject to develop colon cancer. The nucleic acid sequence alterations may encode a variant of the respective protein with impaired or diminished biologic activity, which may comprise DNA repair activity, and diminished DNA repair may enhance the presence of gamma-H2AX foci. In some aspects, the methods comprise determining whether a nucleic acid comprising the ERCC6, WRN, TERT, SHPRH and/or FAAP100 gene obtained from a subject comprises an alteration in the nucleic acid sequence that has been associated with predisposing a subject to develop colon cancer. In some detailed aspects, the methods comprise comparing nucleic acid sequences. For example, such methods may comprise the steps of comparing the sequence of a nucleic acid comprising the ERCC6, WRN, TERT, SHPRH and/or FAAP100 gene obtained from a tissue sample obtained from a subject with one or more reference nucleic acid sequences comprising one or more alterations in the ERCC6, WRN, TERT, SHPRH and/or FAAP100 germline sequence that predispose a subject to genomic instability, and determining whether the ERCC6, WRN, TERT, SHPRH and/or FAAP100 gene sequence obtained from the subject has the alteration based on the comparison. The comparing step may be carried out using a processor programmed to compare nucleic acid sequences, for example, to compare the nucleic acid sequences obtained from the subject and the reference nucleic acid sequences. The methods may optionally include the step of determining the sequence of the nucleic acid comprising the ERCC6, WRN, TERT, SHPRH and/or FAAP100 gene obtained from the subject. The methods may comprise the step of diagnosing whether the subject has a predisposition to genomic instability and/or has a predisposition to develop colon cancer based on the presence or absence of an alteration associated with a predisposition to genomic instability and/or to develop colon cancer in the nucleic acid comprising the ERCC6, WRN, TERT, SHPRH and/or FAAP100 gene obtained from the subject.

From the subject, the sample may be from any tissue or cell in which genomic DNA or a genomic DNA sequence may be obtained. Non-limiting examples include blood, hair, and buccal tissue or cells. The methods may include the step of obtaining the tissue sample, and may include the step of obtaining the nucleic acid, and may include the step of obtaining a cell nucleus. The nucleic acid may be any nucleic acid that has, or from which may be determined, the presence and/or quantity of genomic instability or double stranded DNA breaks, and the cell or nucleus may be any cell or nucleus that has, or from which may be determined, the presence and/or quantity of gamma-H2AX foci. The nucleic acid may be any nucleic acid that has, or from which may be obtained, the germline nucleic acid sequence of the ERCC6, WRN, TERT, SHPRH and/or FAAP100 genes, or the complement thereof, or any portion thereof. For example, the nucleic acid may be chromosomal or genomic DNA, may be mRNA, or may be a cDNA obtained from the mRNA. The sequence of the nucleic acid may be determined using any sequencing method suitable in the art.

In some detailed aspects, the methods comprise hybridizing nucleic acids. For example, such methods may comprise the steps of contacting (preferably under stringent conditions), a nucleic acid comprising the ERCC6, WRN, TERT, SHPRH and/or FAAP100 gene obtained from the subject with one or more polynucleotide probes that have a nucleic acid sequence complementary to an ERCC6, WRN, TERT, SHPRH and/or FAAP100 nucleic acid sequence having one or more alterations that predispose a subject to develop colon cancer, and determining whether the one or more probes hybridized with the nucleic acid comprising the ERCC6, WRN, TERT, SHPRH and/or FAAP100 gene obtained from the subject. The methods may comprise the step of diagnosing whether the subject has a predisposition to develop colon cancer based on whether the probes have hybridized with the nucleic acid.

The probes may comprise a detectable label. The nucleic acid obtained from a subject may be labeled with a detectable label. Detectable labels may be any suitable chemical label, metal label, enzyme label, fluorescent label, radiolabel, or combination thereof. The methods may comprise detecting the detectable label on probes hybridized with the nucleic acid comprising the ERCC6, WRN, TERT, SHPRH and/or FAAP100 gene. The probes may be affixed to a support, such as an array. For example, a labeled nucleic acid obtained from a subject may be contacted with an array of probes affixed to a support. The probes may include any probes described or exemplified herein.

In some detailed aspects, the hybridization may be carried out in situ, for example, in a cell obtained from the subject. For example, the methods may comprise contacting (preferably under stringent conditions) a cell comprising a nucleic acid comprising the ERCC6, WRN, TERT, SHPRH and/or FAAP100 gene obtained from the subject, or contacting (preferably under stringent conditions) a nucleic acid in the cell, with one or more polynucleotide probes comprising a nucleic acid sequence complementary to a ERCC6, WRN, TERT, SHPRH and/or FAAP100 germline nucleic acid sequence having one or more alterations that predispose a subject to develop colon cancer and determining whether the one or more probes hybridized with the nucleic acid comprising the ERCC6, WRN, TERT, SHPRH and/or FAAP100 gene in the cell. The methods may comprise the step of diagnosing whether the subject has a predisposition to develop colon cancer based on whether the probes have hybridized with the nucleic acid. The probes may comprise a detectable label, and the method may comprise detecting the detectable label on probes hybridized with the nucleic acid comprising the ERCC6, WRN, TERT, SHPRH and/or FAAP100 gene. Detectable labels may be any suitable chemical label, metal label, enzyme label, fluorescent label, radiolabel, or combination thereof.

In any of the hybridization assays, the probes may be DNA or RNA, are preferably single stranded, and may have any length suitable for avoiding cross-hybridization of the probe with a second target having a similar sequence with the desired target. Suitable lengths are recognized in the art as from about 20 to about 60 nucleotides optimal for many hybridization assays (for example, see the Resequencing Array Design Guide available from Affymetrix: http://www.affymetrix.com/support/technical/byproduct.affx?product=cseq), though any suitable length may be used, including shorter than 20 or longer than 60 nucleotides. It is preferred that the probes hybridize under stringent conditions to the ERCC6, WRN, TERT, SHPRH and/or FAAP100 nucleic acid sequence of interest. It is preferred that the probes have 100% complementary identity with the target sequence.

The methods described herein, including the hybridization assays, whether carried out in vitro, on an array, or in situ, may be used to determine any alteration in the ERCC6, WRN, TERT, SHPRH and/or FAAP100 germline nucleic acid sequence that has a known or suspected association with predisposing a subject to genomic instability and/or to develop colon cancer, including any of those described or exemplified herein. In any of the methods described herein, the alterations may be, for example, a mutation or variation in the germline nucleic acid sequence relative to a germline nucleic acid sequence that has no known or suspected association with predisposing a subject to develop colon cancer. The alteration may comprise one or more nucleotide substitutions, an addition of one or more nucleotides in one or more locations, a deletion of one or more nucleotides in one or more locations, an inversion or other DNA rearrangement, or any combination thereof. A substitution may, but need not, change the amino acid sequence of the protein encoded by the ERCC6, WRN, TERT, SHPRH and/or FAAP100 gene. Any number of substitutions, additions, or deletions of nucleotides are possible. The alteration may occur in an intron, an exon, or both.

The one or more alterations in the ERCC6 gene may be located in human chromosome 10, for example, at segment 10q11.2. One non-limiting example of a particular alteration that may predispose a subject to develop colon cancer includes an A to T substitution in exon 3. The substitution may occur at position 50,408,777 of human chromosome 10, and may comprise an A to T substitution at this position. The substitution may comprise a polynucleotide having the nucleic acid sequence of SEQ ID NO:1. The polynucleotide having the substitution may comprise SEQ ID NO:1, or a portion thereof. The substitution may occur in the polynucleotide at the position corresponding to position 537 of SEQ ID NOs:1 or 2, and may comprise an A to T substitution at this position. The substitution may occur in the polynucleotide at the position corresponding to position 692 of the mRNA nucleic acid sequence of Accession No. NM_{—}000124 (SEQ ID NO: 3), and may comprise an A to T substitution at this position.

The ERCC6 gene encodes the Cockayne Syndrome B protein (CSB protein). Thus, in some aspects, one or more alterations in the ERCC6 gene may change the amino acid sequence of the CSB protein. One non-limiting example of a particular amino acid alteration that may predispose a subject to develop colon cancer includes an asparagine to tyrosine substitution at position 180 in the CSB protein. The amino acid alteration may comprise a polypeptide having the amino acid sequence of SEQ ID NO:4. The amino acid alteration may comprise a substitution of tyrosine with asparagine in the position corresponding to position 180 in the CSB protein sequence of SEQ ID NO:5. In some aspects, nucleic acid alterations in the ERCC6 gene encode a tyrosine at position 180 in the CSB protein. Thus, the methods may comprise determining whether a nucleic acid comprising the ERCC6 gene obtained from the subject encodes a tyrosine at position 180 of the CSB protein.

The one or more alterations in the WRN gene may be located in human chromosome 8, for example, at segment 8p12. One non-limiting example of a particular alteration that may predispose a subject to develop colon cancer includes a C to T substitution in exon 19. The substitution may occur at position 31,088,698 of human chromosome 8, and may comprise a C to T substitution at this position. The substitution may comprise a polynucleotide having the nucleic acid sequence of SEQ ID NO:6. The polynucleotide having the substitution may comprise SEQ ID NO:6, or a portion thereof. The substitution may occur in the polynucleotide at the position corresponding to position 2113 of SEQ ID NOs:6 or 7, and may comprise a C to T substitution at this position. The substitution may occur in the polynucleotide at the position corresponding to position 2902 of the mRNA nucleic acid sequence of Accession No. NM_{—}000553 (SEQ ID NO:8), and may comprise a C to T substitution at this position.

Another non-limiting example of a particular WRN gene alteration that may predispose a subject to develop colon cancer includes a C to A substitution in exon 19. The substitution may occur at position 31,134,481 of human chromosome 8, and may comprise a C to A substitution at this position. The substitution may comprise a polynucleotide having the nucleic acid sequence of SEQ ID NO:11. The polynucleotide having the substitution may comprise SEQ ID NO:11, or a portion thereof. The substitution may occur in the polynucleotide at the position corresponding to position 3875 of SEQ ID NOs:11 or 7, and may comprise a C to A substitution at this position. The substitution may occur in the polynucleotide at the position corresponding to position 4663 of the mRNA nucleic acid sequence of Accession No. NM_{—}000553 (SEQ ID NO:8), and may comprise a C to A substitution at this position.

In some aspects, the WRN gene may include both the C to T alteration at position 31,088,698 of human chromosome 8 and the C to A alteration at position 31,134,481 of human chromosome 8. The dual substitution may comprise a polynucleotide having the nucleic acid sequence of SEQ ID NO:13. The polynucleotide having the substitution may comprise SEQ ID NO:13, or a portion thereof. The dual substitution may occur in the polynucleotide at the position corresponding to position 2113 and position 3875 of SEQ ID NO:6, 7, or 11, and may comprise a C to T substitution at position 2113 and a C to A substitution at position 3875. The dual substitution may occur in the polynucleotide at the position corresponding to position 2902 and the position corresponding to position 4663 of the mRNA nucleic acid sequence of Accession No. NM_{—}000553 (SEQ ID NO:8), and may comprise a C to T substitution at position 2902 and a C to A substitution at position 4663.

The WRN gene encodes the Werner protein. Thus, in some aspects, one or more alterations in the WRN gene may change the amino acid sequence of the Werner protein. One non-limiting example of a particular amino acid alteration that may predispose a subject to develop colon cancer includes a threonine to isoleucine substitution at position 705 in the Werner protein. The amino acid alteration may comprise a polypeptide having the amino acid sequence of SEQ ID NO:9. The amino acid alteration may comprise a substitution of threonine with isoleucine in the position corresponding to position 705 in the Werner protein sequence of SEQ ID NO:10. In some aspects, nucleic acid alterations in the WRN gene encode an isoleucine at position 705 in the Werner protein. Thus, the methods may comprise determining whether a nucleic acid comprising the WRN gene obtained from the subject encodes an isoleucine at position 705 of the Werner protein.

Another non-limiting example of a particular amino acid alteration that may predispose a subject to develop colon cancer includes a serine to tyrosine substitution at position 1292 in the Werner protein. The amino acid alteration may comprise a polypeptide having the amino acid sequence of SEQ ID NO:12. The amino acid alteration may comprise a substitution of serine with tyrosine in the position corresponding to position 1292 in the Werner protein sequence of SEQ ID NO:10. In some aspects, nucleic acid alterations in the WRN gene encode a tyrosine at position 1292 in the Werner protein. Thus, the methods may comprise determining whether a nucleic acid comprising the WRN gene obtained from the subject encodes a tyrosine at position 1291 of the Werner protein.

In some aspects, two or more alterations in the Werner protein amino acid sequence may predispose a subject to develop colon cancer. For example, the altered Werner protein amino acid sequence may comprise a threonine to isoleucine substitution at position 705 and a serine to tyrosine substitution at position 1292 of the Werner protein. The amino acid alteration may comprise a polypeptide having the amino acid sequence of SEQ ID NO:14. The amino acid alteration may comprise a substitution of threonine with isoleucine at position 705 and a substitution of serine with tyrosine at position 1292 in the Werner protein sequence of SEQ ID NO:10. In some aspects, nucleic acid alterations in the WRN gene encode both an isoleucine at position 705 and a tyrosine at position 1292 in the Werner protein. Thus, the methods may comprise determining whether a nucleic acid comprising the WRN gene obtained from the subject encodes an isoleucine at position 705 of the Werner protein and determining whether a nucleic acid comprising the WRN gene obtained from the subject encodes a tyrosine at position 1292 of the Werner protein.

The one or more alterations in the TERT gene may be located in human chromosome 5, for example, at segment 5p15.3. One non-limiting example of a particular alteration that may predispose a subject to develop colon cancer includes a G to C substitution in exon 2. The substitution may occur at position 1,347,409 of human chromosome 5, and may comprise a G to C substitution at this position. The substitution may comprise a polynucleotide having the nucleic acid sequence of SEQ ID NO:15. The polynucleotide having the substitution may comprise SEQ ID NO:15, or a portion thereof. The substitution may occur in the polynucleotide at the position corresponding to position 591 of SEQ ID NOs:15 or 16, and may comprise a G to C substitution at this position. The substitution may occur in the polynucleotide at the position corresponding to position 650 of the mRNA nucleic acid sequence of Accession No. NM_{—}198253 (SEQ ID NO:17), and may comprise a G to C substitution at this position.

The TERT gene encodes the Telomerase Reverse Transcriptase protein. Thus, in some aspects, one or more alterations in the TERT gene may change the amino acid sequence of the Telomerase Reverse Transcriptase protein. One non-limiting example of a particular amino acid alteration that may predispose a subject to develop colon cancer includes an glycine to arginine substitution at position 198 in the Telomerase Reverse Transcriptase protein. The amino acid alteration may comprise a polypeptide having the amino acid sequence of SEQ ID NO:18. The amino acid alteration may comprise a substitution of glycine with arginine in the position corresponding to position 198 in the amino acid sequence of SEQ ID NO:19. In some aspects, nucleic acid alterations in the TERT gene encode an isoleucine at position 198 in the Telomerase Reverse Transcriptase protein. Thus, the methods may comprise determining whether a nucleic acid comprising the TERT gene obtained from the subject encodes an arginine at position 198 of the Telomerase Reverse Transcriptase protein.

The FAAP100 gene (also known as C17Orf70) encodes the Fanconi anemia-associated protein of 100 kD. Thus, in some aspects, one or more alterations in the FAAP100 gene may change the amino acid sequence of the Fanconi anemia-associated protein of 100 kD. One non-limiting example of a particular amino acid alteration that may predispose a subject to develop colon cancer includes a serine to leucine substitution at position 466 in the Fanconi anemia-associated protein of 100 kD. The amino acid alteration may comprise a polypeptide having the amino acid sequence of SEQ ID NO:23. The amino acid alteration may comprise a substitution of serine with leucine in the position corresponding to position 466 in the Fanconi anemia-associated protein of 100 kD sequence of SEQ ID NO:24. In some aspects, nucleic acid alterations in the FAAP100 gene encode a leucine at position 466 in the Fanconi anemia-associated protein of 100 kD. Thus, the methods may comprise determining whether a nucleic acid comprising the FAAP100 gene obtained from the subject encodes a leucine at position 466 of the Fanconi anemia-associated protein of 100 kD.

The one or more alterations in the FAAP100 gene may be located in human chromosome 17, for example, at segment 77124711. One non-limiting example of a particular alteration that may predispose a subject to develop colon cancer includes a C to T substitution in exon 4. The substitution may occur at position 77,124,711 of human chromosome 17, and may comprise a C to T substitution at this position. The substitution may comprise a polynucleotide having the nucleic acid sequence of SEQ ID NO:20. The polynucleotide having the substitution may comprise SEQ ID NO:20, or a portion thereof. The substitution may occur in the polynucleotide at the position corresponding to position 1397 of SEQ ID NO:20, and may comprise a C to T substitution at this position. The substitution may occur in the polynucleotide at the position corresponding to position 1443 of the mRNA nucleic acid sequence of Accession No. BC_{—}117141 (SEQ ID NO:22), and may comprise a C to T substitution at this position.

In some aspects, one or more alterations in the SHPRH gene may change the amino acid sequence of the SNF2 histone linker PHD RING finger helicase protein. One non-limiting example of a particular amino acid alteration that may predispose a subject to develop colon cancer includes an arginine or histidine to cysteine substitution at position 1184 in the SHPRH protein. The amino acid alteration may comprise a polypeptide having the amino acid sequence of SEQ ID NO:28. The amino acid alteration may comprise a substitution of arginine or histidine with cysteine in the position corresponding to position 1184 in the SHPRH sequence of SEQ ID NO:29 (arg) or SEQ ID NO:30 (his). In some aspects, nucleic acid alterations in the SHPRH gene encode a cysteine at position 1184 in the SHPRH protein. Thus, the methods may comprise determining whether a nucleic acid comprising the SHPRH gene obtained from the subject encodes a cysteine at position 1184 of the SHPRH protein.

The one or more alterations in the SHPRH gene may be located in human chromosome 6, for example, at the SNP rs 148401398. One non-limiting example of a particular alteration that may predispose a subject to develop colon cancer includes a C to T substitution in the coding strand, which may occur at position 146,243,968 of human chromosome 6, and may comprise a G to A substitution in the coding strand complement. The substitution may comprise a polynucleotide having the nucleic acid sequence of SEQ ID NO:25. The polynucleotide having the substitution may comprise SEQ ID NO:25, or a portion thereof. The substitution may occur in the polynucleotide at the position corresponding to position 3950 of SEQ ID NO:26, and may comprise a C to T substitution at this position. The substitution may occur in the polynucleotide at the position corresponding to position 3949 of the mRNA nucleic acid sequence of Accession No. NM_{—}001042683 (SEQ ID NO:27), and may comprise a C to T substitution at this position.

The reference nucleic acid sequences used in nucleic acid sequence comparison aspects of the methods may comprise one or more of SEQ ID NO:1, SEQ ID NO:6, SEQ ID NO:11, SEQ ID NO:13, SEQ ID NO:15, SEQ ID NO:20, and SEQ ID NO:25, or portion thereof having one or more alterations associated with a predisposition/risk of developing colon cancer. The reference nucleic acid sequences may also include nucleic acid sequences that do not have any nucleotide alterations that are associated with a predisposition/risk of developing colon cancer to serve as controls in the comparison, or for determinations that the subject does not have a germline nucleic acid sequence alteration that predisposes to develop colon cancer. Non-limiting examples of nucleic acid sequences without such alterations include SEQ ID NO:2, SEQ ID NO:3, SEQ ID NO:7, SEQ ID NO:8, SEQ ID NO:16, SEQ ID NO:17, SEQ ID NO:21, SEQ ID NO:22, SEQ ID NO:26, and SEQ ID NO:27. Reference nucleic acid sequences having any portion of the sequence of these sequence identifiers may be used.

The polynucleotide probes used in nucleic acid hybridization aspects may comprise a portion of one or more of SEQ ID NO:1, SEQ ID NO:6, SEQ ID NO:11, SEQ ID NO:13, SEQ ID NO:15, SEQ ID NO:20, and SEQ ID NO:25, the portion containing the genomic instability and/or colon cancer risk-associated alteration. The nucleic acid sequence of the probes may be complementary to the relevant portion of SEQ ID NO:1, SEQ ID NO:6, SEQ ID NO:11, SEQ ID NO:13, SEQ ID NO:15, SEQ ID NO:20, or SEQ ID NO:25.

Polynucleotide probes having a nucleic acid sequence without any alterations associated with a predisposition to develop genomic instability and/or colon cancer may be used to serve as controls in hybridization assays, or for determinations that the subject does not have a germline nucleic acid sequence alteration that predisposes to genomic instability or colon cancer. Non-limiting examples of nucleic acid sequences without an alteration, from which such probes may be derived, include SEQ ID NO:2, SEQ ID NO:3, SEQ ID NO:7, SEQ ID NO:8, SEQ ID NO:16, SEQ ID NO:17, SEQ ID NO:21, SEQ ID NO:22, SEQ ID NO:26, and SEQ ID NO:27, and the probes may be obtained from the regions of these sequences where the respective alteration is located. The probe nucleic acid sequence may be complementary to the appropriate portion of SEQ ID NO:2, SEQ ID NO:3, SEQ ID NO:7, SEQ ID NO:8, SEQ ID NO:16, SEQ ID NO:17, SEQ ID NO:21, SEQ ID NO:22, SEQ ID NO:26, and SEQ ID NO:27.

The methods for diagnosing, whether based on sequence comparison or probe hybridization, may further comprise the steps of treating the subject with a regimen capable of inhibiting the onset of colon cancer. These steps may be included, for example, if it is determined that the subject has a predisposition to develop colon cancer. In some aspects, the treatment regimen may comprise administering to the subject an effective amount of the CSB, Werner, Telomerase Reverse Transcriptase protein, Fanconi anemia associated protein of 100 kD, SNF2 histone linker PHD RING finger helicase protein, or genes that encode these proteins in vectors that can integrate and express in tissue stem cells. In some aspects, the treatment regimen comprises administering to the subject an effective amount of a compound or pharmaceutical composition capable of delaying or inhibiting the onset of colon cancer. In some aspects, the treatment regimen comprises one or more of diet management, vitamin supplementation, nutritional supplementation, exercise, psychological counseling, social counseling, education, and regimen compliance management. In some aspects, the treatment regimen comprises administering to the subject an effective amount of a compound or pharmaceutical composition that enhances the activity of one or more of the CSB protein, the Werner protein, the Telomerase Reverse Transcriptase protein, SNF2 histone linker PHD RING finger helicase protein, and the Fanconi anemia associated protein of 100 kD.

In the diagnostic methods, the tissue sample obtained from the subject may be from any tissue in which replicating cells and/or a genomic DNA sequence may be obtained. Non-limiting examples include blood, hair, and buccal tissue. Blood may comprise peripheral blood lymphocytes (PBLs). The methods may include the step of obtaining the tissue sample, and may include the step of obtaining the nucleic acid. The nucleic acid may be any nucleic acid that has, or from which may be obtained, the germline nucleic acid sequence for the ERCC6, WRN, TERT, SHPRH and/or FAAP100 genes, or the complement thereof, or any portion thereof. For example, the nucleic acid may be chromosomal or genomic DNA, may be mRNA, or may be a cDNA obtained from the mRNA.

The diagnostic methods are preferably based on determining alterations in the germline nucleic acid sequences of the ERCC6, WRN, TERT, SHPRH, and FAAP100 genes that predispose a subject having such alterations to develop colon cancer, including any of the alterations described or exemplified herein. The reference nucleic acid sequences and the probes are thus based on alterations that predispose to develop colon cancer, and based on control sequences that do not have alterations that predispose to develop colon cancer.

The invention also provides isolated polynucleotides comprising a nucleic acid comprising the ERCC6, WRN, TERT, SHPRH and/or FAAP100 gene and having one or more alterations that predispose a subject to develop colon cancer. The invention also provides isolated polynucleotides comprising a probe having a nucleic acid sequence complementary to a nucleic acid sequence having one or more alterations in the ERCC6, WRN, TERT, SHPRH and/or FAAP100 gene that predispose a subject to develop colon cancer. Probes may have any suitable number of nucleotide bases. The one or more alterations may be any of the alterations described or exemplified herein. The probes preferably hybridize to a nucleic acid comprising the ERCC6, WRN, TERT, SHPRH and/or FAAP100 gene under stringent conditions

Polynucleotides include polyribonucleotides and polydeoxyribonucleotide, which may be unmodified RNA or DNA or modified RNA or DNA, and include single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. Polynucleotides may have triple-stranded regions comprising RNA or DNA or both RNA and DNA, modified bases, unusual bases such as inosine, modified backbones, and enzymatic or metabolic modifications.

The alterations may comprise, for example, a nucleic acid sequence encoding a tyrosine at position 180 of the CSB protein. The CSB protein may comprise SEQ ID NO:4. A nucleic acid sequence encoding a tyrosine at position 180 of the CSB protein may comprise an A to T substitution in the codon encoding an asparagine at position 180 of the CSB protein, and the A to T substitution may occur at a position corresponding to position number 50,408,777 in the ERCC6 gene locus on human chromosome number 10.

The alterations may comprise, for example, a nucleic acid sequence encoding an isoleucine at position 705 of the Werner protein. The Werner protein may comprise SEQ ID NO:9. A nucleic acid sequence encoding an isoleucine at position 705 of the Werner protein may comprise a C to T substitution in the codon encoding a threonine at position 705 of the Werner protein, and the C to T substitution may occur at a position corresponding to position number 31,008,698 in the WRN gene locus on human chromosome number 8. In addition to, or in the alternative to a nucleic acid sequence encoding an isoleucine at position 705 of the Werner protein, the alteration may comprise a nucleic acid sequence encoding a tyrosine at position 1292 of the Werner protein. The Werner protein may comprise SEQ ID NO:12 or SEQ ID NO:14. A nucleic acid sequence encoding a tyrosine at position 1292 of the Werner protein may comprise a C to A substitution in the codon encoding a serine at position 1292 of the Werner protein, and the C to A substitution may occur at a position corresponding to position number 31,134,481 in the WRN gene locus on human chromosome number 8.

The alterations may comprise, for example, a nucleic acid sequence encoding an arginine at position 198 of the Telomerase Reverse Transcriptase protein. The Telomerase Reverse Transcriptase protein may comprise SEQ ID NO:18. A nucleic acid sequence encoding an arginine at position 198 of the Telomerase Reverse Transcriptase protein may comprise a G to C substitution in the codon encoding a serine at position 198 of the Telomerase Reverse Transcriptase protein, and the G to C substitution may occur at a position corresponding to position number 1,347,409 in the TERT gene locus on human chromosome number 5.

The alterations may comprise, for example, a nucleic acid sequence encoding a leucine at position 466 of the Fanconi anemia associated protein of 100 kD. The Fanconi anemia associated protein of 100 kD may comprise SEQ ID NO:23. A nucleic acid sequence encoding a leucine at position 466 of the Fanconi anemia associated protein of 100 kD may comprise a C to T substitution in the codon encoding a serine at position 466 of the Fanconi anemia associated protein of 100 kD, and the C to T substitution may occur at a position corresponding to position number 77,124,711 in the FAAP100 gene locus on human chromosome number 17.

The alterations may comprise, for example, a nucleic acid sequence encoding a cysteine at position 1184 of the SNF2 histone linker PHD RING finger helicase protein. The SNF2 histone linker PHD RING finger helicase protein may comprise SEQ ID NO:29 or SEQ ID NO:30. A nucleic acid sequence encoding a cysteine at position 1184 of the SNF2 histone linker PHD RING finger helicase protein may comprise a C to T substitution in the codon encoding an arginine at position 1184 of the SNF2 histone linker PHD RING finger helicase protein, and the C to T substitution may occur at a position corresponding to position number 146,243,968 in the SHPRH gene locus on human chromosome number 6.

The invention also features a support comprising a plurality of polynucleotides comprising a nucleic acid sequence, or portion thereof, comprising the ERCC6, WRN, TERT, SHPRH and/or FAAP100 genes and having one or more alterations in the nucleic acid sequence that predispose a subject to develop colon cancer, and optionally, a plurality of polynucleotides comprising a nucleic acid sequence, or portion thereof, comprising the ERCC6, WRN, TERT, SHPRH and/or FAAP100 genes and not having any alterations in the nucleic acid sequence that are known to predispose a subject to develop colon cancer. The support may comprise an array. The polynucleotides may be probes. The probes may comprise a portion of the nucleic acid sequence of SEQ ID NO:1, SEQ ID NO:6, SEQ ID NO:11, SEQ ID NO:15, SEQ ID NO:20, or SEQ ID NO:25 comprising an alteration associated with predisposing a subject to genomic instability and/or to develop colon cancer, and the alteration may comprise any alteration described or exemplified herein. The probes may comprise the complement of the portion of the nucleic acid sequence of SEQ ID NO:1, SEQ ID NO:6, SEQ ID NO:11, SEQ ID NO:15, SEQ ID NO:20, or SEQ ID NO:25 comprising an alteration associated with predisposing a subject to genomic instability and/or to develop colon cancer.

The invention also features isolated polypeptides, including isolated proteins comprising a polypeptide having an amino acid sequence encoded by a polynucleotide comprising one or more alterations that predispose a subject to develop colon cancer. Polypeptides include polymers of amino acid residues, one or more artificial chemical mimetic of a corresponding naturally occurring amino acid, as well as to naturally occurring amino acid polymers and non-naturally occurring amino acid polymers.

The polypeptides may comprise the CSB protein comprising a tyrosine at position 180. The polypeptides may comprise the Werner protein comprising an isoleucine at position 705. The polypeptides may comprise the Werner protein comprising a tyrosine at position 1292. The polypeptides may comprise the Werner protein comprising an isoleucine at position 705 and a tyrosine at position 1292. The polypeptides may comprise the Telomerase Reverse Transcriptase protein comprising an arginine at position 198. The polypeptides may comprise the Fanconi anemia associated protein of 100 kD comprising a leucine at position 466. The polypeptides may comprise the SNF2 histone linker PHD RING finger helicase protein comprising a cysteine at position 1184. The polypeptides may comprise an amino acid sequence encoded by the nucleic acid sequence of SEQ ID NO:1, SEQ ID NO:6, SEQ ID NO:11, SEQ ID NO:15, SEQ ID NO:20, or SEQ ID NO:25. The polypeptides may comprise the amino acid sequence of SEQ ID NO:4, SEQ ID NO:9, SEQ ID NO:12, SEQ ID NO:18, SEQ ID NO:23, or SEQ ID NO:28.

The invention also features systems for diagnosing a predisposition to develop colon cancer. In general, the systems comprise a data structure comprising one or more reference nucleic acid sequences having one or more alterations in ERCC6, WRN, TERT, SHPRH and/or FAAP100 gene associated with predisposing a subject to develop colon cancer, and a processor operably connected to the data structure. Optionally, the data structure may comprise one or more reference nucleic acid sequences that do not have any alterations in the ERCC6, WRN, TERT, SHPRH and/or FAAP100 genes associated with a predisposition of a subject to develop colon cancer. The processor is preferably capable of comparing, and preferably programmed to compare determined nucleic acid sequences (for example, those determined from nucleic acids obtained from a subject) with reference nucleic acid sequences.

The reference nucleic acid sequences may comprise the one or more alterations described or exemplified herein. For example, the alterations may comprise a nucleic acid sequence encoding a tyrosine at position 180 of the CSB protein. The alterations may comprise a nucleic acid sequence encoding an isoleucine at position 705 of the Werner protein and/or a nucleic acid sequence encoding a tyrosine at position 1292 of the Werner protein. The alterations may comprise a nucleic acid sequence encoding an arginine at position 198 of the Telomerase Reverse Transcriptase protein. The alterations may comprise a nucleic acid encoding a leucine at position 466 of the Fanconi anemia associated protein of 100 kD. The alterations may comprise a nucleic acid encoding a cysteine at position 1184 of the SNF2 histone linker PHD RING finger helicase protein. The reference nucleic acid sequences may comprise the nucleic acid sequence of one or more of SEQ ID NO:1, SEQ ID NO:2, SEQ ID NO: 6, SEQ ID NO:7, SEQ ID NO:11, SEQ ID NO:13, SEQ ID NO:15, SEQ ID NO:16, SEQ ID NO:20, SEQ ID NO:21, SEQ ID NO:25, or SEQ ID NO:26.

Optionally, the system may comprise an input for accepting determined nucleic acid sequences obtained from tissue samples from a subject. Optionally, the system may comprise an output for providing results of a sequence comparison to a user such as the subject, or a technician, or a medical practitioner. Optionally, the system may comprise a sequencer for determining the sequence of a nucleic acid such as a nucleic acid obtained from a subject. Optionally, the system may comprise a detector for detecting a detectable label on a nucleic acid.

Optionally, the system may comprise computer readable media comprising executable code for causing a programmable processor to determine a diagnosis of the subject, for example whether the subject has a predisposition to develop colon based on whether or not a nucleic acid obtained from the subject includes a sequence alteration associated with a predisposition to develop colon cancer. The diagnosis may be based on the comparison of determined nucleic acid sequences with reference nucleic acid sequences. The diagnosis may be based on a determination of hybridization of a nucleic acid probe with a nucleic acid obtained from the subject. Thus, the system may comprise an output for providing a diagnosis to a user such as the subject, or a technician, or a medical practitioner. Optionally, the system may comprise computer readable media that comprises executable code for causing a programmable processor to recommend a treatment regimen for the subject, for example, a treatment regimen for preventing, inhibiting, or delaying the onset of colon cancer.

In any of the systems, a computer may comprise the processor or processors used for determining information, comparing information and determining results. The computer may comprise computer readable media comprising executable code for causing a programmable processor to determine a diagnosis of the subject. The systems may comprise a computer network connection, including an Internet connection.

The invention also provides computer-readable media. In some aspects, the computer-readable media comprise executable code for causing a programmable processor to compare the nucleic acid sequence of the ERCC6, WRN, TERT, SHPRH and/or FAAP100 gene determined from a nucleic acid obtained from a tissue sample obtained from a subject with one or more reference nucleic acid sequences having one or more alterations in the ERCC6, WRN, TERT, SHPRH and/or FAAP100 gene sequence associated with predisposing a subject to develop genomic instability and/or to develop colon cancer. The alterations may be any alteration described or exemplified herein. Optionally, the computer-readable media comprise executable code for causing a programmable processor to compare the nucleic acid sequence of the ERCC6, WRN, TERT, SHPRH and/or FAAP100 gene determined from a nucleic acid obtained from a tissue sample obtained from a subject with one or more reference nucleic acid sequences that do not have any alterations in the ERCC6, WRN, TERT, SHPRH and/or FAAP100 gene sequence associated with predisposing a subject to genomic instability and/or to develop colon cancer. The computer readable media may comprise a processor, which may be a computer processor.

The reference nucleic acid sequences may comprise any of the one or more alterations described or exemplified herein. For example, the alterations may comprise a nucleic acid sequence encoding a tyrosine at position 180 of the CSB protein. The alterations may comprise a nucleic acid sequence encoding an isoleucine at position 705 of the Werner protein and/or a nucleic acid sequence encoding a tyrosine at position 1292 of the Werner protein. The alterations may comprise a nucleic acid sequence encoding an arginine at position 198 of the Telomerase Reverse Transcriptase protein. The alterations may comprise a nucleic acid encoding a leucine at position 466 of the Fanconi anemia associated protein of 100 kD. The alterations may comprise a nucleic acid encoding a cysteine at position 1184 of the SNF2 histone linker PHD RING finger helicase protein. The reference nucleic acid sequences may comprise the nucleic acid sequence of one or more of SEQ ID NO:1, SEQ ID NO:2, SEQ ID NO: 6, SEQ ID NO:7, SEQ ID NO:11, SEQ ID NO:13, SEQ ID NO:15 SEQ ID NO:16, SEQ ID NO:20, SEQ ID NO:21, SEQ ID NO:25, or SEQ ID NO:26.

The systems and computer readable media may be used in any of the methods described or exemplified herein, for example, methods for diagnosing a predisposition to develop colon cancer. For example, the systems and computer readable media may be used to facilitate comparisons of gene sequences, or to facilitate a diagnosis.

The methods, systems, and computer readable media comprise various reference values. For example, the reference values comprise certain quantities such as a quantity of gamma-H2Ax or a quantity of double stranded DNA breaks, and comprise certain qualities such as the presence or absence of a type of polymorphism in a gene sequence or the presence or absence of a type of genomic instability such as chromosomal aneuploidy. In general, such reference values may be established according to studies of individuals and/or studies of populations. It is contemplated that, over time, as more and more individuals and larger populations are studied, the reference values, particularly the quantitative reference values, may become more precise or established to have a greater confidence. Reference value quantities may comprise quantities based on available information for any given period of time.

The following examples are provided to describe the invention in greater detail. They are intended to illustrate, not to limit, the invention.

### Example 1

### Identification of Sequence Alterations Relevant to Colon Cancer Predisposition

It is believed that dysfunction of genes that maintain genome stability underlies a substantial fraction of familial colorectal carcinoma (FCRC). Based on this hypothesis, preliminary studies utilized colorectal carcinoma (CRC) patients in an in-house Gastrointestinal Cancer Risk Assessment Program who met the following criteria: (1) they developed CRC before the age of 50 and/or had a first degree relative with colon cancer and, (2) had tested negative for Lynch Syndrome/Hereditary Non-Polyposis Colorectal Cancer (HNPCC) by standard tests of their tumor for microsatellite instability and/or immunohistochemistry for levels of mismatch repair proteins and tested negative for Familial Adenomatous Polyposis coli by having fewer than five polyps detected by colonoscopy. Many of these patients had clinical features atypical for MUTYH polyposis.

All patients in the Program had donated peripheral blood from which buffy coat white blood cells (WBCs) were frozen in dimethylsulfoxide (DMSO) and used to prepare genomic DNA and had signed a broad consent for research, and Controls were selected from the same BioSample Repository that had no personal history of cancer or cancer in a first degree relative, and were matched by sex and age. Lymphocytes were cultured from eight independent patients, using stimulation with phytohemagluttinin (PHA) and Interleukin 2 (IL-2). Seven samples yielded enough cells to generate metaphase spreads, and several of these yielded enough cells to evaluate by flow cytometry.

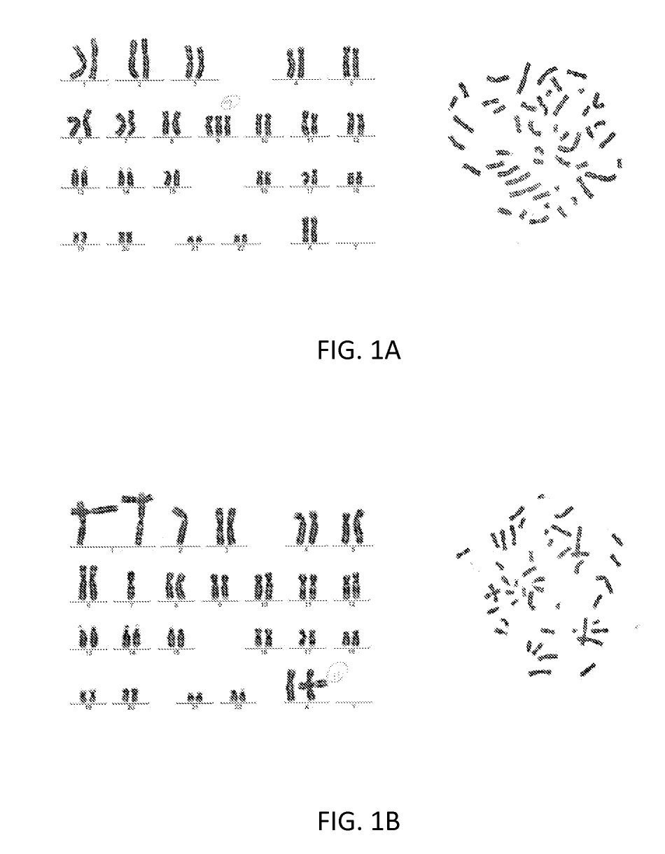

Metaphase spreads were generated from proliferating cultures by addition of colcemid, swelling in hypotonic buffer, and dropping from height onto a slide. Chromosomes were stained with Giemsa stain to identify them. At least 50 well-separated spreads with condensed chromosomes from all 7 patients and 3 controls were scored by standard clinical cytogenetics criteria for any notable abnormality, including premature chromatid separation, aneuploidy, and chromosomal rearrangements. One patient, number 120713, showed 4 out of 50 spreads with chromosomal gains (8%; gains are viewed as more reliable than losses), each different (FIG. 1A and FIG. 1B, which show 2 of the 4 spreads). This was an unusually high degree of aneuploidy.

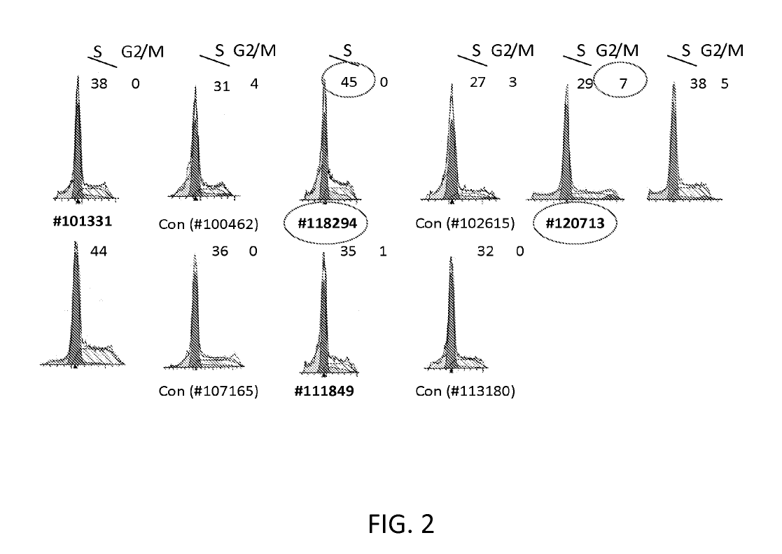

Only one chromosomal gain was seen in the remaining 6 cases examined (0.2%) and only 3 of 3 controls (1%), consistent with a published mode frequency of gains in normal lymphocytes of 1.3% (Cimino M C et al. (1986) Mutat. Res. 167:107-22). A second patient, number 118294, showed a complex chromosomal rearrangement. Flow cytometry of propidium-iodide-stained cells show the highest level of S phase in this patient, among 3 other cases and 5 controls (FIG. 2). Flow cytometry of cells from patient 120713 showed the highest level of G2/M phase, among the cases and controls. On this basis, patients 120713 and 118294 were selected for further analysis.

Exome sequencing was performed on their peripheral blood DNAs by SeqWright Services (Texas). The library size was good and >85% of target sequences had >20× coverage. All sequence variants were initially screened by eye for potential involvement in cell replication, DNA repair, cell cycle checkpoints or mitosis and the severity of the molecular change. The following uninformative sequence variants were found: (1) non-sense changes in 120713: EFCAB3, C22orf30, SELP, C2orf65, PRAMEEF1, ULK4, and ZNF571; and non-sense changes in 118294: FAM83A, ZNF5858, C17orf58, and ALKBH4; and (2) internal deletions or splice site changes in 120713: FAM113A, C14orf13, MED13L, PDGFD, and HERPUD2; and internal deletions or splice site changes in 118294: TRPM3, FAM113A, FLJ41603, SPEN, PASK, GAPVD1, SOX1. None of these changes affected proteins with known roles in cell replication and/or genome stability. In addition, patient 120713 displayed 265 missense variants, and patient 118292 displayed 262 missense variants. Among these affected genes, several had roles in replication and/or genome stability: for patient 120713: ERCC6, WRN, CDKN1a, and DUB3, and for patient 118294: TERT, WRN, and EXO1.

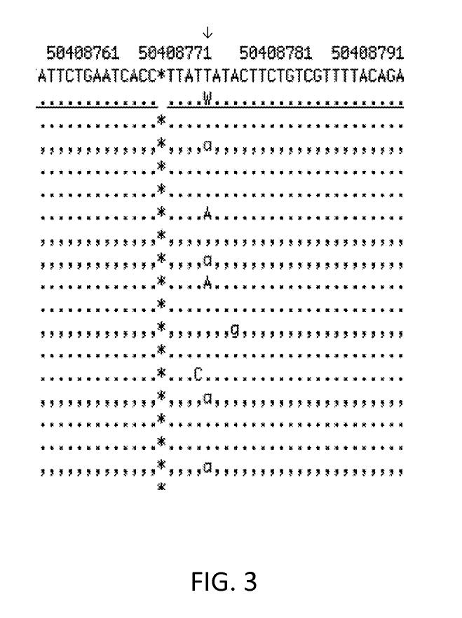

Among the missense variants in these latter genes, it is believed that the variants in ERCC6 and TERT had not been previously reported in publicly available single nucleotide polymorphism (SNP) databases (National Heart Lung and Blood Institute Exome Sequencing Project server (http://evs.gs.washington.edu/EVS/). The sequence quality in these regions was verified as excellent by direct inspection, and the reads were unambiguously assigned (FIG. 3).

ERCC6 is a chromatin remodeling protein (CSB protein) that is implicated in transcription-coupled DNA repair. Homozygous inherited inactivating proteins in ERCC6 cause Cockayne syndrome, a growth disorder associated with sensitivity to ultraviolet light. Polymorphisms within the ERCC6 gene have been statistically associated with head and neck tumors, bladder carcinoma, and lung cancer in other studies. The likelihood that these variants degrade protein function was evaluated using Consurf (University of Massachusetts) and PolyPhen2 (PP2; Harvard University) software.

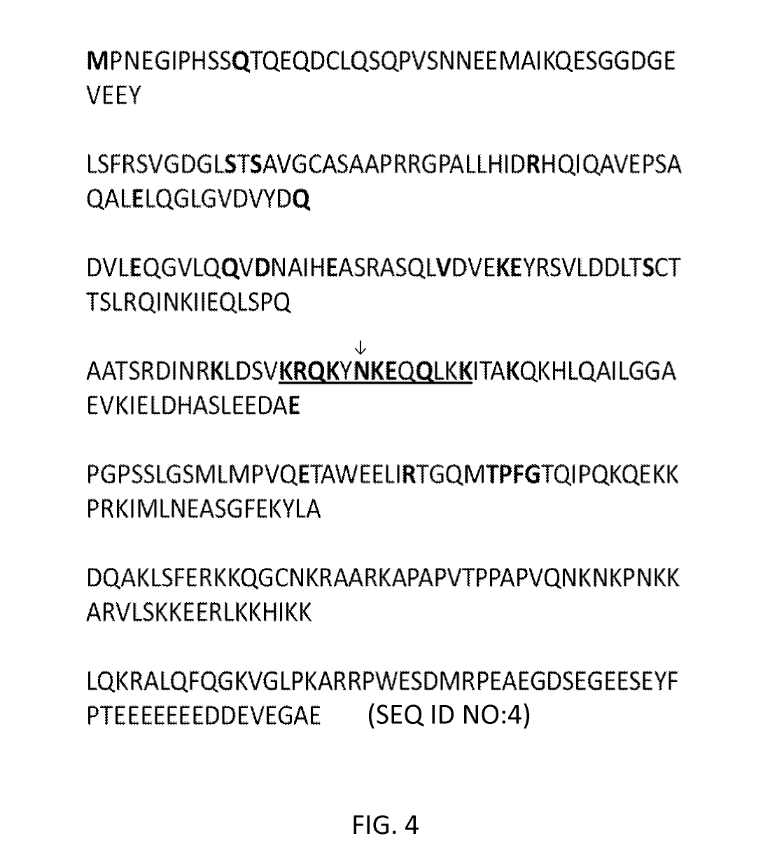

The ERCC6 (CSB) protein variant, N180Y, represents in a tyrosine substitution for asparagine, a non-conservative change in a residue that is completely conserved among vertebrate ERCC6-encoded proteins. Furthermore, this residue is within a stretch of 9 highly conserved residues (FIG. 4) that comprise a coiled-coil motif.

The PP2 program predicted the variant to be probably damaging to function of the protein, with its highest possible confidence score of 1.0. It is believed that the coiled-coil motif currently has no known ascribed function. However, the amino-terminal 400 amino acids have been implicated in three important biochemical function of ERCC6: intramolecular inhibition of ATPase activity, inhibition of non-specific DNA binding, and interaction with the transcription complex. It is believed that the coiled-coil motif is the strongest region of sequence conservation in this region of the protein. These motifs are thought to mediate protein-protein interactions. Therefore, this motif is a logical candidate region to mediate one or more of these biochemical functions.

Examination of the clinical history of patient 120713 revealed four characteristics that may be caused by ERCC6 deficiency: (1) This patient has a history of colon cancer at age 48, without the polyposis of APC or MUTYH diseases or the microsatellite instability or mismatch repair protein expression abnormalities of Lynch. Somatic ERCC6 gene mutations have recently been found in genome-wide sequencing studies in 6% of CRCs (Wood L D et al. (2007) Science 318:110843; and Network CGA. (2012) Nature 487:330-7). This frequency was notable, but did not reach statistical significance, and ERCC6 was not classified as a ‘driver’. It is believed that ERCC6 may contribute to the development of both sporadic CRC and FCRC. (2) The patient developed basal cell carcinoma (BCC) at the unusually early age of 23. The patient's brother developed BCC at age 50 and both the patient's mother and father had multiple BCCs in their 40s. Although Cockayne Syndrome patients do not develop BCC, their cells are particularly sensitive to UV light; BCC is believed to be a highly UV-driven tumor. Moreover, mice with inherited ERCC6 mutations are prone to UV-induced skin tumors. (3) The patient developed macular degeneration (MD) at an unusually early age (in her 40's). A sequence polymorphism in ERCC6 has been linked to MD, although this association was not confirmed in two follow-up studies. (4) The patient's father developed bladder carcinoma at age 62. Somatic ERCC6 mutations have recently been reported in some bladder cancers in Southeast Asia. Thus, there are potential links to ERCC6 dysfunction in the patient's history of colon cancer, BCC, MD, and family history of bladder cancer. These observations are suggestive of an inherited constitutional predisposition to cancer and degenerative disease with features of ERCC6 dysfunction.

WRN is a helicase that plays an important role in DNA repair, although the mechanisms of repair remain under active investigation. Mutations in WRN cause Werner's syndrome, a growth disorder associated with features of premature aging. Regions of the protein that help form the helicase domain have been mapped. The variant in the Werner protein from patient 120713 is T705I. This variant is within the helicase domain, and was predicted by the PolyPhen2 program to be probably damaging, with a high confidence score (>0.9). The WRN variant from patient 118294 is S1292Y. This variant was scored by the PP2 program as being possibly damaging.

TERT is required to maintain telomeres at chromosome ends, thereby preventing them from causing chromosomal rearrangements and being recognized as damaged DNA. TERT mutations cause progressive diseases including aplastic anemia and pulmonary fibrosis. Progress has been made in identifying regions of TERT that contribute to its RNA-directed DNA polymerase activity and its interaction with protein partners. The TERT variant from patient 118294 is G198R. It was predicted by PolyPehn2 to be possibly damaging.

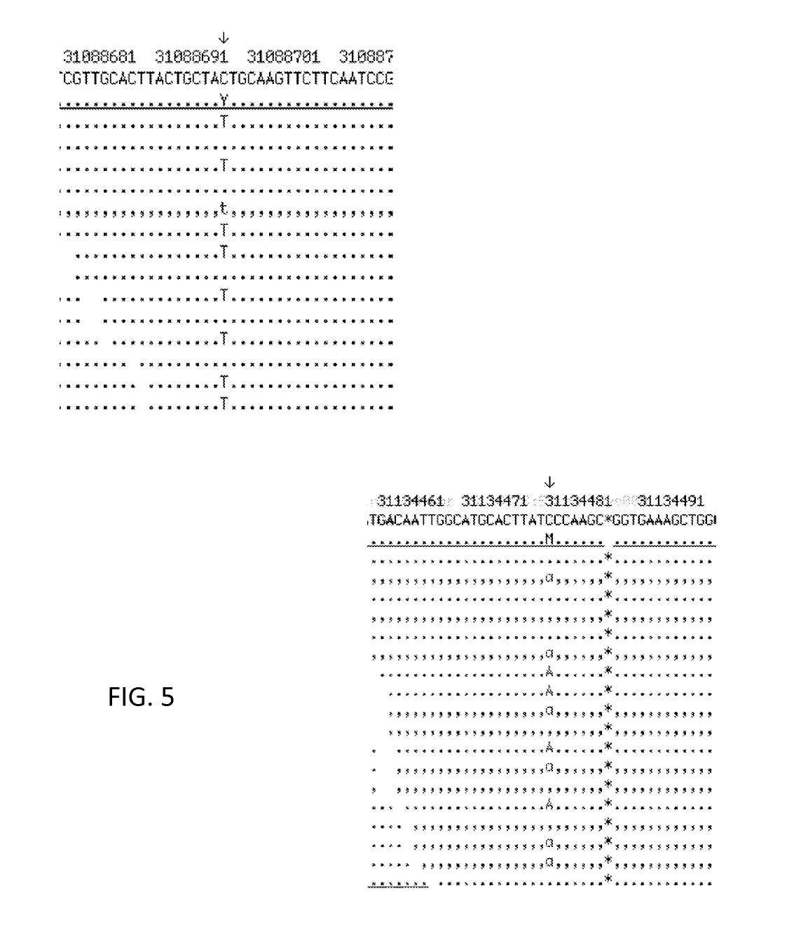

The ERCC6 N180Y, WRN T705I, TERT G198R, and FAAP100 S466L (see more below) variants were each confirmed by direct polymerase chain reaction (PCR) amplification of patient DNA and Sanger DNA sequencing (FIG. 5). The other potential variants were excluded on the basis of sequence changes that were either common (e.g., >1/1000) in SNP databases and/or predicted to represent benign changes in the encoded proteins.

This analysis of variants was repeated more rigorously by identifying all genes in Gene Ontology (GO) consortium databases to be associated with the terms DNA replication, DNA repair, checkpoint, mitosis, or mitotic. Thirty four variants in patient 118294 and nineteen variants in patient 120713 were associated with these GO terms. These variants were loaded into the PP2 program by batch methods; analysis was by a somewhat more stringent version of the program. Variants were then excluded that were present at a frequency less than 20% in the exome sequencing reads (and, therefore, unreliably constitutional), present in the NHLBI SNP databases at frequencies >1/1000, or predicted to likely be benign by the PP2 program.

ERCC6 N180Y and WRN T705I were again the two leading candidate variants that emerged from this analysis, with PP2 scores >0.99. A new top-tier candidate variant emerged from this analysis: FAAP100/C17Orf70 S466L, with a PP@ score >0.98. Te FAAP100 protein was recently identified as an essential component of the Fanconi's Anemia DNA repair pathway (see below). Additional candidates emerged from this analysis which were designated as ‘second tier’ because they manifested higher SNP frequencies, lower PP2 scores, and/or carried less evidence for direct involvement in genome stability. From patient 120713: TRERF1, a transcription factor that may regulate the mitotic spindly checkpoint (1/13005 in SNP databases, PP2 score of 0.99); DYNC1H1, a protein implicated in mitotic spindle organization (not present in SNP databases, PP2 score 0.93 (probably damaging)); TRPM1, a transcription factor implicated in the DNA damage checkpoint (not present in SNP databases, PP2 score 0.90 (possibly damaging)), and SMC1B, a mediator of chromosomal condensation (not present in SNP databases, PP2 score possibly damaging).

The GO gene analysis from patient 118294 demoted the TERT variant to probably benign by PP2 analysis and yielded three new candidate variants: PTPRT, a protein tyrosine phosphatase that is mutated somatically in a fraction of CRCs (not present in SNP databases, probably damaging by PP2); TBRG4, protein that drives yeast cells into the cell cycle (5/13005 in SNP databases, probably damaging by PP2), and CDC14A, a phosphatase implicated in mitotic anaphase (not present in SNP databases, possibly damaging by PP2). Thus, none of the variants in patient 118294, including TERT, are believed to be top-tier.

Given that each of the second tier variants from patient 120713 and the CDC14 variant from patient 118294 has a direct or indirect role in regulating mitosis, the next stages of investigation will include an interrogation of the efficiency of mitosis in cells from each patient. Isolated cells will be infected with a retrovirus encoding a green fluorescent protein (GFP)-histone H2B fusion protein, and chromosome dynamics during mitosis will be observed in living cells. These experiments are somewhat technically challenging, given the small size of lymphocytes and the fact that they generally do not adhere to tissue culture dish bottoms, but preliminary experiments are underway.

In summary, eight independent FCRC cases were screened for constitutional genomic instability (CGI) by analyzing metaphase spreads and flow cytometry-generated cell cycle profiles of cultured peripheral lymphocytes. Two patients showed evidence of CGI in the form of aneuploidy (patient #120713), a chromosomal rearrangement (patient #118294), and/or increased fractions of cells within replicative phases (both patients). Exome sequencing revealed novel or rare heterozygous sequence variants in relevant genes. 120713 has a novel variant in ERCC6/CSB, a nucleotide excision repair gene. The variant is a strong candidate for being causal: it encodes a non-conservative change in a highly conserved residue in a region of the protein with biochemically-defined functions. The patient harboring this allele has three other clinical conditions consistent with ERCC6 dysfunction. Each patient also has a rare sequence variant in WRN, a DNA repair helicase. 120713 also carries a rare sequence variant in FAAP100, a scaffolding protein of the Fanconi's anemia DNA repair pathway. These observations provide evidence that ERCC6 and possibly WRN contribute to CGI and colon cancer in these FCRC cases.

### Example 2

### Follow Up Studies

The studies described in Example 1 suggest that constitutional genomic instability is more widespread than currently recognized. It is believed that heterozygous mutations will be functionally important, due to haplo-insufficiency and/or dominant negative effects. Currently recognized FCRC syndromes are autosomal dominant at the organismic level, but are thought to be largely recessive at the cellular level. The following describes additional experiments to be undertaken.

Studies will evaluate whether the sequence variants of the ERCC6, WRN, TERT, and FAAP100 genes described in this specification inactivate the function of the proteins they encode. It is believed that dysfunction of genes that maintain genome stability underlies a substantial fraction of FCRC. These studies will proceed along the following basic outline: (1) Test whether the sequence variants inactivate protein function by (a) introducing the sequence variants into expression vectors by site-directed mutagenesis, (b) testing whether the variant proteins fail to rescue cellular deficiencies in the respective proteins, and (c) testing whether the variant proteins exert dominant negative effects; (2) Further define the nature and severity of CGI in the FCRC patients by (a) repeating metaphase spread and flow cytometry assays on primary cells, (b) performing assays for activation of the DNA damage response on primary cells, (c) establishing immortalized lymphocytes from the patients and assess their expression of the variant proteins and CGI, (d) testing whether patient cells are hypersensitive to exogenous DNA damage, and (e) test whether cell phenotypes can be rescued by exogenous expression of candidate genes; and, (3) Screen 30 additional FCRC patients for CGI and relevant sequence variants by (a) examining metaphase spreads, cell cycle profiles, and DNA damage foci in peripheral lymphocytes, and (b) perform exome sequencing in patients with evidence for CGI.

It is believed that these studies will provide new molecular insights into causes of FCRC and CGI and functional elements of DNA repair proteins while offering new methods to screen for predisposition to colon cancer and to diagnose affected members of FCRC families in pre-clinical stages. This capability should allow intensive colon cancer screening by endoscopy to be focused on those patients who should benefit strongly and to be avoided in those who will not. Related clinical conditions, such as predisposition to basal cell carcinoma, macular degeneration, and bladder cancer, may also be better managed.

(1) Testing Whether the Sequence Variants Inactivate Protein Function.

(a) Introduce the Sequence Variants into Expression Vectors by Site-Directed Mutagenesis.

The investigation will begin with introducing the sequence variants into expression vectors encoding the wild type proteins. The vectors have already been prepared, and expression experiments are underway.

### (b) Test Whether the Variant Proteins Fail to Rescue Cellular Deficiencies in the Respective Proteins.

ERCC6 deficient cells have been established from patients with Cockayne's syndrome and are being maintained in culture. These cells are sensitive to UV treatment, consistent with the known role of ERCC6 in DNA repair. This phenotype can be rescued by expression of the wild type protein, providing a convenient assay system for protein function. As an initial test of ERCC6 function, the wild type and variant protein from patient 120713 will be expressed in parallel in the cognate deficient cells, and these proteins will be assayed to determine whether the variant fails to restore resistance to UV irradiation. The ability of ERCC6 to complement UV sensitivity likely integrates several biochemical activities of the protein and provides a good screen for functionally important defects. To further define the molecular defect, the wild type and variant proteins will be expressed in mammalian cells, and nuclear extracts will be prepared from these cells. These extracts will then be incubated with chromatin prepared from untreated or UV-irradiated cells. The UV-induced chromatin binding of the proteins will be compared. The protein will also be expressed in bacteria with an epitope tag, and the purified protein will be assayed for ATPase activity on DNA templates. Additional experiments may be suggested by these assays. These biochemical assays might also reveal a defect that failed to be detected during overexpression of the protein in the assays of UV sensitivity.

WRN-deficient cells have been established from patients with Werner's syndrome and are being maintained in culture. However, the most straightforward test of WRN function is to test its helicase activity, the activity central to WRN function in DNA repair. This activity is most readily tested by purifying the protein from bacterial extracts and incubating it with short double-stranded oligonucleotides with single-stranded 5′ ends. WRN will unwind these templates, an activity readily detected by a shift in mobility on non-denaturing gel electrophoresis. The activities of wild type and variant WRN protein will be tested in this assay.

Most primary cells are TERT-deficient and can be infected with the retroviral vector. The wild type and variant TERT protein will be expressed in parallel, and telomerase activity will be evaluated in vitro using a standard assay.

FAAP100 acts as a scaffold upon which BRCA1 and other DNA repair proteins concentrate at lesions, to activate Chk1 and degrade Ccdc25A, among other functions. We will compare the ability of wild type and variant FAAP100 proteins to perform these actions.

### (c) Test Whether the Variant Proteins Exert Dominant Negative Effects.

Defective proteins that occupy limited sites where the protein must normally act can exert dominant negative effects. It is believed that in some cases, expression of a defective protein disrupts function of the remaining wild type protein. Such sites may be homo- or hetero-multimeric complexes involving the protein. There is some evidence that ERCC6 multimerizes. This is also true for WRN. TERT must function as a complex with a small RNA that templates synthesis of telomeric DNA. In addition, TERT interacts with a small set of proteins that protect telomeres from recognition by the DNA damage pathway. As a scaffolding protein, FAAP100 may sequester other proteins involved in DNA damage responses, including DNA repair and cell cycle arrest.

These experiments will test whether expression of the ERCC6 variant protein confers sensitivity to UV irradiation. The variant will be titrated in co-transfections with limiting amounts of vector that rescues UV sensitivity of Cockayne syndrome cells, and the extent to which expression of the variant restores sensitivity or is inert will be assessed.

As well, whether the WRN variant confers sensitivity to the topoisomerase I poison camptothecin will be investigated. WRN syndrome cells do not show increased sensitivity to UV, but demonstrate distinctly increased apoptosis during S phase following exposure to this drug. The detailed mechanism is unknown, but the drug is known to trap topo I on DNA and to involve inhibition of transcription during S phase. It is thought to potentially reflect an inability of the WRN helicase to resolve and repair collisions between RNA polymerase complexes and/or DNA polymerase complexes and protein-modified DNA, with resulting double strand DNA breaks. Camptothecin doses of 20-50 nM cause S phase delay and a 5-6-fold increase in apoptosis of Werner cells.

It is believed that the ERCC6 N180Y variant will disrupt protein function, given the constellation of clinical findings in patient 120713 consistent with ERCC6 dysfunction, the evidence that the variant residue is likely damaging, and the critical roles played by amino-terminal region the protein. The variant is anticipated to help unravel the function of the central motif in this region, the coiled-coil domain motif.

For example, follow-up studies will compare intramolecular and extrinsic protein-protein interactions mediated by this domain and disrupted by the variant (e.g., with the carboxy-terminal protein and transcription complex, by ‘pull-down’ assays, etc.) and will test whether the variant exhibits the marked conformational change thought to occur with lesion-induced activation of ATPase activity. Most extant ERCC6 mutations in Cockayne's syndrome and engineered mutations compromise the ATPase activity of ERCC6.

Whether the variant may be haploinsufficient or dominant negative is more difficult to predict. It is evident that patient 120713 did not have full-blown Cockayne syndrome, so the variant does not entirely inactivate ERCC6 function. Cockayne syndrome carriers are heterozygous for ERCC6 mutations. There is some evidence for phenotypes in their cells, such as modest UV sensitivity, but little clinical data addressing relevant diseases. If the variant ablates inhibition of ATPase activity of the protein, it may bind more indiscriminately and remodel chromatin structure in deleterious ways. It may, thereby, potentially alter transcription and/or divert repair factors, exerting dominant negative effects not seen with standard Cockayne syndrome mutations that inactivate ATPase activity. This molecular mechanism provides a possible alternative explanation for potential dominant negative effects of the variant without compromise of an ERCC6 homopolymeric complex.

It is believed that the WRN variant in patient 120713 will also inactivate protein function, and is predicted to be probably damaging. This variant may therefore compromise DNA repair is a second way in patient 120713, with additive or synergistic effects. Neoplasia is present in both maternal and paternal lineages of the patient, suggesting that there may be independent gene variants that predispose to neoplasia in the pedigree. However, if cell lines may be established, they will be tested for whether they exhibit major ongoing genetic instability and whether complementation with wild type ERCC6, WRN, or both are needed to restore genome stability.

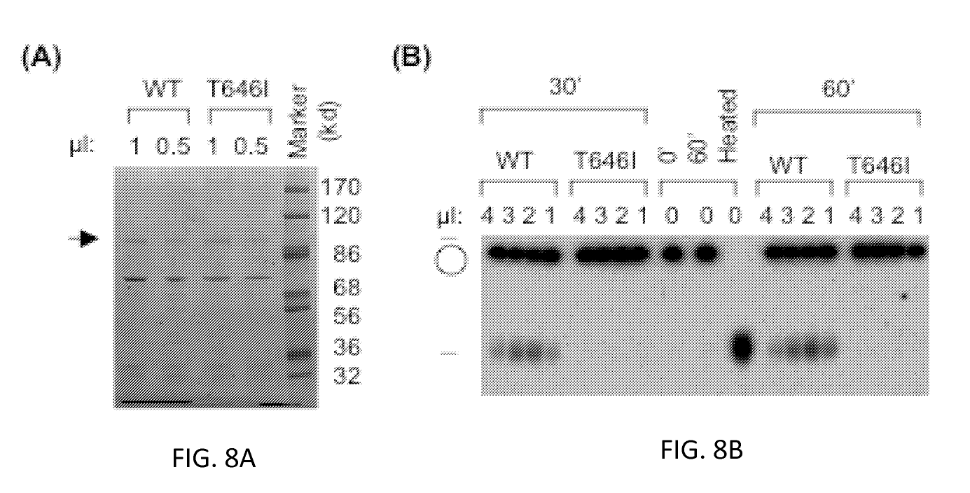

Initial experiments have uncovered direct biochemical evidence that patient 120713 WRN variant T646I inactivates helicase activity of the protein (FIG. 8A and FIG. 8B). The WRN helicase domain is highly conserved between humans and Xenopus. The homologous patient mis-sense variant was introduced into the Xenopus WRN helicase domain, which has been used routinely in helicase assays. The recombinant wild type and T646I variant were produced in bacteria, purified, and assayed for helicase activity using a ³²P-labeled oligonucleotide.

The expressed wild type and variant T646I proteins were stained with Coomassie blue (FIG. 8A, arrow) in two different dilutions. The purified proteins were assayed for helicase activity in vitro for 30 or 60 minutes using a ³²P-labeled oligonucleotide annealed to a longer single stranded DNA molecule. The oligonucleotide dissociated from the complex by helicase activity (FIG. 8B, upper line and circle, left-most column) migrated more rapidly (FIG. 8B, lower line). The T646I variant had no detectable helicase activity as shown by the absence of the dissociated radio-labeled oligonucleotide in the T646I tests, either at 30 minutes or 60 minutes. Similar results were observed in two other experiments using two independent protein preparations (data not shown).

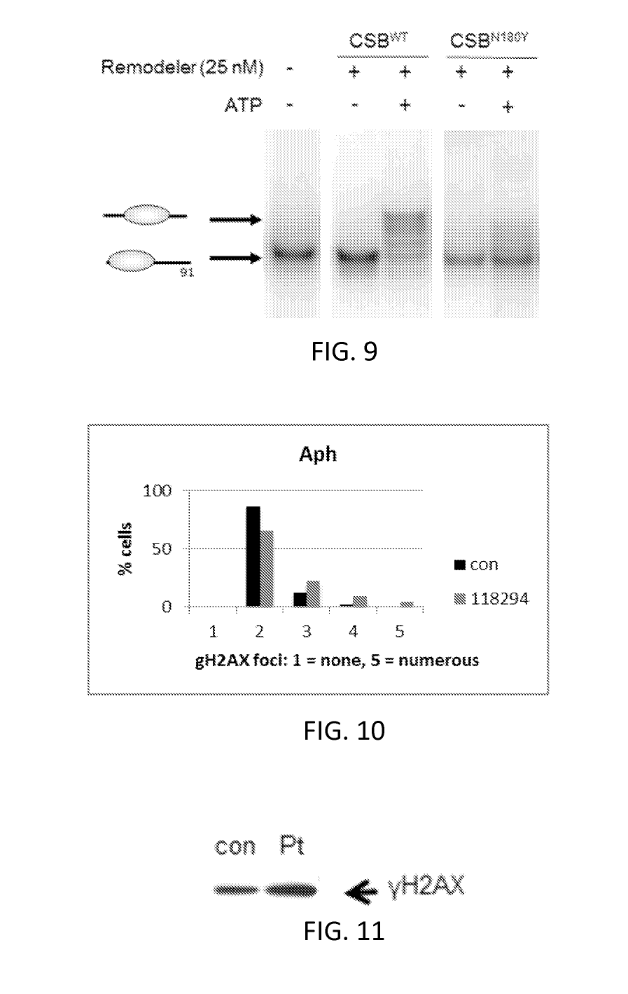

Initial experiments also uncovered direct biochemical evidence that patient 120713 ERCC6 variant N180Y reduces chromatin remodeling activity of the CSB protein (FIG. 9). The recombinant wild type CSB and N180Y variant were expressed in bacteria, and the chromatin remodeling activities of the purified proteins were assayed in vitro using a ³²P-labeled nucleotide with bound nucleosome.

The 32P-labeled end-positioned mononucleosomes (FIG. 9, lower band) were used as substrates in vitro for recombinant CSB wild-type and CSB N180Y variant proteins, and subjected to polyacrylamide gel electrophoresis. The N180Y variant exhibited markedly reduced remodeling activity relative to the wild type protein.

These data (reduced activity of Werner and CSB proteins upon amino acid sequence alteration due to a gene alteration) identify two candidate gene variants to account for genomic instability and colon cancer risk. The WRN and ERCC6 variants, and potentially also the FAAP100 variant, are believed to be responsible for genomic instability, as evidenced by the genomic instability and cancer predisposition of patient 120713 and patient 120713's family.

(2) Further Examining Cells for Evidence of CGI.

The presence of 4 chromosomal gains in 50 metaphase spreads (8%), from patient 120713 is unlikely to represent a chance occurrence in normal cells. This rate of gains greatly exceeds the published rate of gains seen in normal stimulated lymphocytes (mode 0.4%) and the rate observed in the rest of the case and control samples in this study (0.7%). Gains are considered more reliable than losses, as the latter are sometimes artifacts of chromosome spreading. However, gains in well-separated spreads such as these are typically not technical artifacts. The spreads were generated by an in-house Genomics Facility, which has extensive experience with this method and performs it routinely for clinical analysis. Nonetheless, these assays will be repeated on cell lines established from patient 120713 and controls, to further validate the CGI and more accurately determine its level.

Patient 118294 exhibited a complex chromosomal rearrangement. This event cannot be artifactual, as it must be formed within the cell and is a rare event in normal cells. However, it is desired to gauge more accurately the rate of such events in cells from this patient. This patient also demonstrated the highest S phase fraction of any sample tested. The difference (14% above the mean S phase fraction in control samples) is well beyond the normal technical variation in S phase fraction in such samples (ca. 2-3%).

Generation of metaphase spreads and flow cytometry cell cycle profiles is useful for screening patients for CGI. However, the nature and severity of CGI in such cells have not been fully defined. Most GI is associated with double strand DNA breaks. Low levels of such lesions are difficult to detect directly. Nonetheless, their presence can often be detected indirectly by detecting activation of the DNA damage response (DDR). This response involves the concentration of repair proteins around the lesions, forming what is termed DNA damage foci. These foci are commonly visualized by immunofluorescence. Markers of DDR will be tested to identify this response in patients 120713 and 118294, by immunofluorescence (IF; most sensitive), immunohistochemistry (IHC; readily performed in most clinical pathology labs), and immunoblotting (IB; most specific for histone variant γH2AX).

(a) Repeating Metaphase Spread and Flow Cytometry Assays on Excess Primary Cells.

These experiments will verify and better quantitate the rate of generation of chromosomal and cell cycle abnormalities in patients 118294 relative to controls. Cultured cells will be stimulated with PHA. Some will then be treated with the mitotic spindle poison colchicine, permeabilized, dropped onto slides to generate spreads, and stained with Giemsa, to stain chromosomal bands and allow identification of individual chromosomes. At least 50 well-separated chromosome spreads per patient will be scored for aneuploidy and chromosomal rearrangements in triplicate. A portion of each PHA-stimulated culture (at least 100,00 cells) will be fixed in ethanol, stained with propidium iodide, and analyzed by flow cytometry, for DNA content in triplicate. The fraction of cells with S and G2/M phases, respectively, will be compared.

(b) Establishing Immortalized Lymphocytes from the Patients and CGI Assays.

A retroviral TERT vector has been transfected into a packaging cell line, and high titer viral supernatants have been generated. These will be used to infect control cells, to verify the method, and then samples from 120713 and 118294 will be used. T lymphocyte growth will be fostered by addition of IL-2. These polyclonal cultures will be expanded and aliquots frozen in DMSO. Other portions will be used to repeat the metaphase spread and flow cytometry analyses. Finally, a portion of each PHA-stimulate primary cell culture will be infected with retrovirus expressing SV40 large T antigen. These polyclonal cultures will be expanded and frozen in DMSO. In addition, we are preparing Epstein Barr Virus-transformed B lymphocyte cell lines form patient 120713 and controls.

### (c) Performing Assays for DNA Damage Markers.

Primary cells, if available, or immortalized cells will be pelleted by low-speed centrifugation, embedded in histogel, fixed in paraformaldehyde (PFA) or formaldehyde, respectively, and sectioned as per a tissue block. The PFA-fixed material will be subjected to IF for DDR markers. The formalin-fixed material will be subjected to immunohistochemistry for DDR markers. Protein extracts will be prepared from other cells and subjected to immunoblotting for γH2AX. DNA will be damaged in samples of normal cells, as positive controls, using UV- and X-irradiation and treatment with camptothecin.

Given that there is a TERT gene variant in patient 118294, and defective telomerase activity has been linked to ds DNA breaks and genomic instability as well as intestinal tumorigenesis, telomere integrity will be evaluated in this patient. Telomere length will be estimated by in situ hybridization using a probe complementary to the TERT repeat and high-resolution fluorescence microscopy. Telomere-associated DNA damage foci will be assayed in cells fixed with paraformaldehyde by co-immunofluorescence for the telomere protein TRAP1 or TRF1 and DNA damage response markers γH2AX and 53BP1.

### (d) Testing Whether Patient Cells are Hypersensitive to Exogenous DNA Damage.

Cockayne syndrome patients and their cells are hypersensitive to UV-irradiation. Patient 120713 has a personal and family history of basal cell carcinoma, a UV-associated tumor, and a history of macular degeneration, thought to be in part a UV-driven disease. Exogenous damage may elicit a sensitivity that is less apparent in un-treated cells. Cells will be exposed to 4 J/m2 joules of UV-irradiation from a UV lamp and examined for DDR foci. Cells will also be assayed for their long-term proliferative capacity by the colony-outgrowth assay. Similar assays will be performed following X-irradiation and treatment with cisplatin, respectively, as controls for more general defects in cells from patient 120713 and to detect other potential defects in DNA repair and/or the DDR in patient 118294.

(e) Testing Whether Cell Phenotypes can be Rescued by Exogenous Expression of Candidate Genes.

Whether observed patient cell phenotypes of GI, UV sensitivity, camptothecin sensitivity, and telomeric DNA damage foci can be rescued by overexpression of the respective wild types proteins will be tested. It is believed that the repeat assays of CGI will confirm it in the patients and help determine its severity. The results will also clarify whether the CGI differs qualitatively in the two patients. For example, it will be determined whether or not the CGI in patient 120713 primarily causes aneuploidy, without chromosomal rearrangement and whether or not the reverse is true to patient 118194. Although ERCC6 has primarily been implicated in nucleotide excision repair of bulky lesions, which do not necessarily form double strand DNA breaks, bulky lesions or their partially repaired intermediates are thought to often be converted to ds breaks when encountered by replication forks. In addition, ERCC6 has been implicated to lesser degrees in other forms of DNA repair, including homologous recombination, a favored route for repair of ds breaks. It is believed that cells from patient 120713 will be hypersensitive to UV-irradiation. In this case, whether this phenotype can be rescued by overexpression of ERCC6 wild-type more effectively than the variant allele will be investigated. If the WNR allele from this patient also appears to be defective, whether exogenous WRN expression can reduce sensitivity will be investigated.

Initial experiments identified evidence that lymphocytes from patient 118294 form increased double-stranded DNA breaks, at baseline and in response to exposure to DNA damaging agents. Lymphocytes isolated from patient 118294 were treated with aphidicolin for 2 hours, and subject to immunofluorescence staining for gamma-H2AX. One hundred control lymphocytes and one hundred cells from patient 118294 were scored in a blinded fashion for nuclear foci: 0 score=none; 1=many faint or <10 bright; 2=>10 bright; 3=>30 bright; 4=numerous bright; 5=confluent bright (FIG. 10). Patient 118294 showed more gamma-H2AX foci (p=0.002) (Fisher's exact test). Patient 118294 also showed more gamma-H2AX foci at baseline (p=0.08) and with treatment with camptothecin (p=0.006)(data not shown). Immunofluorescence showed greater gamma-H2AX foci in patient cells in response to aphidicolin and camptothecin exposure relative to control cells.

Immunoblotting studies confirmed the greater gamma-H2AX levels (FIG. 11). Chromatin extracts from the cells were normalized for H2AX content, and subject to immunoblotting for gamma-H2AX. Immunoblotting in chromatin extracts from the patient (Pt) was more intense than in normalized extracts from control cells (FIG. 11).

(3) Screen 30 Additional FCC Patients for CGI and Sequence Variants in Related Genes.

These proposed studies will triple the previous patient set and allow for the setting of initial bounds on the frequency of CGI in FCC patients. In addition, candidate genes responsible for the observed CGI have been identified. At this point, each represents a sample size of one. Examination of additional patients will provide for a determination of whether the responsible gene set is small or large. If the current experience can be extrapolated to the additional 30 patients, it is anticipated that more patients with CGI will be identified. These data can be used subsequently to design larger clinical studies to more accurately assess the frequency of involved genes and to assess the practicality of determining the underlying lesions by targeted sequencing of candidate genes, rather than exome sequencing.

Initial experiments screened lymphocytes from 5 additional familial colon cancer patients. Within these studies, lymphocytes from patient 27818 showed greater gamma-H2AX foci relative to the control cells, in response to treatment with aphidicolin (p=0.003) and camptothecin (p<0.001) treatments (data not shown). In addition, lymphocytes from patient 282617 showed greater gamma-H2AX foci relative to the control cells, in response to treatment with ultraviolet light (p<0.001)(data not shown). In total, 4/12 (33%) of familial colon cancer patients exhibited increased gamma-H2AX foci, versus 0/4 controls. These data support the use of gamma-H2AX markers and screening assays with at-risk patients.

### Example 3

### FAAP100 S466L

An additional candidate disease-causing variant in patient 120713 was identified. To systematically analyze the list of gene variants derived from the exome sequencing results, Gene Ontology (GO) consortium databases were used to focus on variant genes associated with the terms DNA replication, DNA repair, checkpoint, mitosis, or mitotic. Thirty four variants in patient 118294 and 19 variants in patient 120713 were associated. Variants were identified that represented >40% of the sequencing reads (and were, therefore, likely to be at least heterozygous), absent from NHLBI SNP databases or present at frequencies <1/1000 (thereby reducing type 1 errors), and predicted by the PolyPhen2 program (Sunyaev, Harvard University) to be probably damaging to protein function. A few were excluded that appeared to not be directed related to CGI, on the basis of being expressed primarily outside the nucleus and/or in a severely restricted tissue pattern. From this analysis, patient 118294 did not yield a strong candidate variant. However, 3 good candidate missense variants were found in patient 120713. In addition to the previously recognized variants ERCC6/CSB N180Y and WRN T705I, C17Orf70/FAAP100 S466L was identified as a strong candidate disease-causing variant.

FAAP100 is an understudied but essential component of the Fanconi's anemia (FA) DNA repair complex. FA is a rare recessive syndrome associated with bone marrow failure, genetic instability, and cancer. It involves a failure to prevent DNA double strand (ds) breaks during DNA replication. FA cells fail to mono-ubiquitinate FANCD2, the central outcome of the pathway, and are very sensitive to DNA cross-linking agents such as mitomycin C. It has recently been established that FANCD is the breast and ovarian cancer tumor suppressor BRCA2, and the complex interacts with BRCA1. FAAP100 acts as a scaffolding protein for the ubiquitin ligase FANCL, but has few defined motifs, and its functional elements have not been mapped. This gene is a potential link to the history of two paternal cousins with early onset breast and ovarian cancers, respectively. If the heterozygous variant compromises the FA pathway, this variant could account for or help account for the patient's apparent defective DNA repair (see next advance), genetic instability, and predisposition to colon cancer.

The FAAP100 variant represents a C to T change (G to A on the opposite strand) at nucleotide 1443 of accession number BC117141 (SEQ ID NO:22). This nucleotide is at position 77124711 on human chromosome 17. The change results in substitution of leucine for serine at amino acid 466 of the protein (SEQ ID NO:23). This substitution is predicted by the PolyPhen2 program to be probably damaging to protein function with high confidence (0.98 score out of 1.00).

### Example 4

### Increase in Double Stranded Breaks and Gamma-H2AX Foci

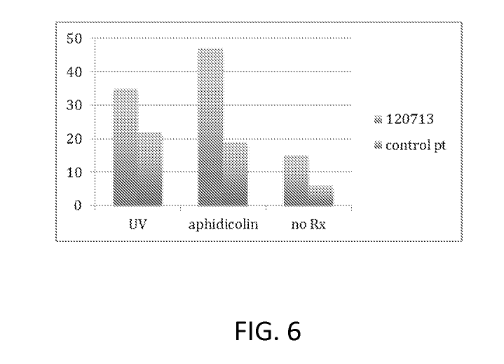

It was determined that patient 120713 exhibited an exagerated response to DNA damage, likely reflecting increased double stranded (ds) DNA breaks. Ds breaks are thought to be a major cause of instability of chromosome structure. The ds break also serves as a nidus for detection of DNA damage responses (DDRs) to a variety of damage, including bulky DNA adducts, intra- and inter-strand cross-links, and collapse of replication forks. Recent data suggest that many ds breaks are formed by replicative events, such as reverse branch migration of Holiday junctions when movement of the DNA replication fork is impaired. Thus, many repair events can result in a ds break. At such breaks, the alternate histone H2AX undergoes extensive phosphorylation, forming ‘γH2AX’ foci visible by immunofluorescence (IF). Other DDR proteins such as phosphorylated ATM/ATR and 53BP1 are recruited into such foci. During work for the project, an in-house Cell Culture Facility worked out conditions under which IL-2, anti-T-cell receptor, and anti-CD3 antibodies stimulate robust growth of primary T-lymphocytes from peripheral blood lymphocytes. In preliminary studies, lymphocytes were treated with ultraviolet light (UV) or the DNA polymerase inhibitor aphidicolin. Aphidicolin is commonly used to reveal DNA repair defects. It generates replicative stress, with collapse of stalled replication forks and generation of ds breaks. The cells were then allowed to adhere to poly-lysine-coated slides, fixed with paraformaldehyde, and stained for γH2AX. Flow cytometry confirmed equivalent fractions of replicating cells in patient 120713 and the control. It was observed that cells from patient 120713 showed substantially greater γH2AX foci in response to treatment with UV or aphidicolin when compared to its age- and sex-matched normal control (FIG. 6; each P<0.001, by Fisher's exact test).

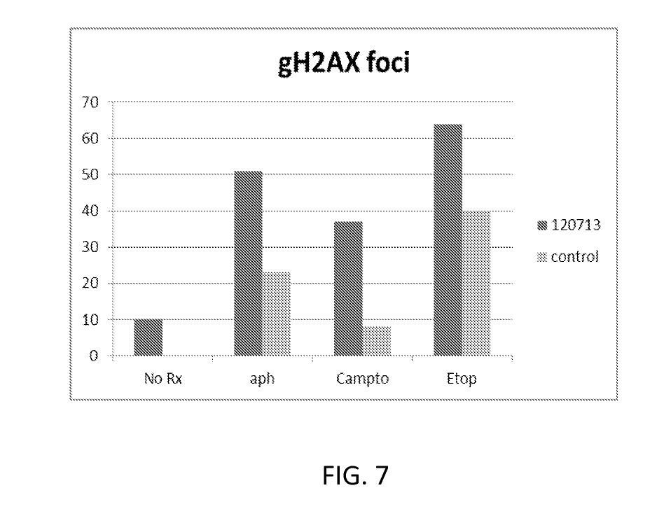

Additional data showed further evidence of a greater DNA damage response, marked by gamma-H2AX foci scored in a blinded fashion, from patient 120713 (FIG. 7). The data show ongoing DNA damage response at the baseline in the patient's lymphocytes (No Rx), as well as in response to treatment with aphidicolin (aph), camptothecin (Campto), and etoposide (Etop). The graph shows that the levels of gamma-H2AX foci are higher in patient 120713 (dark grey) relative to a control subject (light grey).

These findings provide further evidence for a DNA repair defect in patient 120713. Moreover, they offer the prospect that assaying the DDR in normal lymphocytes from at-risk individuals may help identify those with a predisposition to colon cancer. This assay might take the form of immunofluorescence staining for γH2AX, as shown here, or immunohistochemistry, immunoblotting, enzyme-linked immunosorbant assays (ELISAs), or flow cytometry.

### Example 5

### SNF2 Histone Linker PHD RING Finger Helicase Variant

Another gene variant believed to contribute to genomic instability was identified in patient 118284. This variant occurred in the SNF2 histone linker PHD ring finger helicase (SHPRH) gene, and encodes a variant protein SHPRH R1184C. SHPRH is a recently-identified orthologue of S. cerevisiae RAD5, and has regions of homology to both WRN and ERCC6. Without intending to be limited to any particular theory or mechanism of action, it is believed that this homology potentially reflects some functional similarity in the role of SHPRH in DNA repair. In the yeast, RAD5 contributes to ubiquitination of the polymerase clamp-loader PCNA in response to stalling of replication complexes at DNA structural lesions. Acute knockdown of SHPRH causes chromosomal defects, and may have other functions in post-replicative repair.

SHPRH is commonly deleted in tumors, suggesting that it may be a tumor suppressor. SHPRH particularly fosters recovery from treatment with mmc, whereas its homologue helicase-like transcription factor (HLTF) fosters recovery from irradiation. An SHPRH defect fits well with the S-phase delay observed in this patient, as a cause of defective response to polymerase pausing and/or a cause of the S-phase delay itself. R1184 is highly conserved itself and near the middle of a conserved stretch of the primary sequence, but is not within a recognized motif.

A crude molecular model of the SHPRH SNF2 domain structure was generated based on the x-ray crystal structure of the 1Z63 Sulfolobus solfataricus SWI2/SNF2 ATPase Core, with which SHPRH shares extensive homology, though only 19% identity. In this model, R1184 is well positioned to contact DNA (not shown). This residue is stringently conserved as arginine or histidine, consistent with the notion that a positive charge may be needed here to bind the acidic DNA backbone and, support the DNA repair functions of the protein. It is believed that the substituted cysteine (R1184C) may be in position to form a novel disulfide bond with a neighboring cysteine in the tertiary structure. Thus, it is believed that the SHPRH variant R1184C may contribute to genomic instability and cancer.

The invention is not limited to the embodiments described and exemplified above, but is capable of variation and modification within the scope of the appended claims.

We claim:

1. A method for reducing the risk of developing colon cancer in a human subject, comprising,
(a) determining whether peripheral blood lymphoctyes isolated from the subject have elevated levels of gamma-H2AX foci and chromosomal genomic instability, wherein the presence of both elevated levels of gamma-H2AX foci and chromosomal genomic instability indicate that the subject has a risk of developing colon cancer; and
(b) if the subject has a risk of developing colon cancer, treating the subject with a treatment regimen capable of inhibiting the onset of colon cancer.

2. The method of claim 1, further comprising isolating peripheral blood lymphocytes from the subject.

3. The method of claim 1, wherein the chromosomal genomic instability comprises a chromosomal aneuploidy.

4. The method of claim 3, wherein the chromosomal aneuploidy comprises a gain of chromosome 9 or a gain of chromosome 11.

5. The method of claim 1, wherein it is determined that the subject has a risk of developing colon cancer, and the treatment regimen comprises one or more of diet management, vitamin supplementation, nutritional supplementation, exercise, psychological counseling, social counseling, education, and regimen compliance management.

6. The method of claim 1, wherein it is determined that the subject has a risk of developing colon cancer, and the treatment regimen comprises administering to the subject an amount of the Cockayne Syndrome B protein effective to reduce the risk that the patient will develop colon cancer.

7. The method of claim 1, wherein it is determined that the subject has a risk of developing colon cancer, and the treatment regimen comprises administering to the subject an amount of the Werner protein effective to reduce the risk that the patient will develop colon cancer.

8. The method of claim 1, wherein it is determined that the subject has a risk of developing colon cancer, and the treatment regimen comprises administering to the subject an amount of the Telomerase Reverse Transcriptase protein effective to reduce the risk that the patient will develop colon cancer.

9. The method of claim 1, wherein it is determined that the subject has a risk of developing colon cancer, and the treatment regimen comprises administering to the subject an amount of the SNF2 histone linker PHD RING finger helicase protein effective to reduce the risk that the patient will develop colon cancer.

10. The method of claim 1, wherein it is determined that the subject has a risk of developing colon cancer, and the treatment regimen comprises administering to the subject an amount of the Fanconi anemia associated protein of 100 kD effective to reduce the risk that the patient will develop colon cancer.

11. The method of claim 1, wherein it is determined that the subject has a risk of developing colon cancer, and the treatment regimen comprises administering to the subject an amount of the Cockayne Syndrome B protein, Werner protein, Telomerase Reverse Transcriptase protein, SNF2 histone linker PHD RING finger helicase protein, Fanconi anemia associated protein of 100 kD, or a combination thereof, effective to reduce the risk that the patient will develop colon cancer.

12. The method of claim 1, wherein step (a) further comprises isolating gamma-H2AX foci from the peripheral blood lymphocytes and determining the level of one or more of phosphorylated ataxia telangiectasia mutated (ATM), Rad3-related protein (ATR), and Tumor suppressor p53-binding protein 1 (53BP1) in the gamma-H2AX foci, wherein the presence of elevated levels of gamma-H2AX foci and chromosomal genomic instability, and the presence of elevated levels of one or more of ATM, ATR, and 35BP1 in the gamma-H2AX foci indicate that the subject has a risk of developing colon cancer.

13. The method of claim 1, wherein step (a) further comprises isolating a gene encoding the ERCC6 gene from the peripheral blood lymphocytes and determining whether the gene encodes a tyrosine at position 180 of the Cockayne Syndrome B protein, wherein the presence of elevated levels of gamma-H2AX foci and chromosomal genomic instability, and a polymorphism in the ERCC6 gene encoding a tyrosine at position 180 of the Cockayne Syndrome B protein indicate that the subject has a risk of developing colon cancer.

14. The method of claim 1, wherein step (a) further comprises isolating a gene encoding the WRN gene from the peripheral blood lymphocytes and determining whether the gene encodes an isoleucine at position 705 of the Werner protein, wherein the presence of elevated levels of gamma-H2AX foci and chromosomal genomic instability, and a polymorphism in the WRN gene encoding an isoleucine at position 705 of the Werner protein indicate that the subject has a risk of developing colon cancer.

15. The method of claim 1, wherein step (a) further comprises isolating a gene encoding the WRN gene from the peripheral blood lymphocytes and determining whether the gene encodes a tyrosine at position 1292 of the Werner protein, wherein the presence of elevated levels of gamma-H2AX foci and chromosomal genomic instability, and a polymorphism in the WRN gene encoding a tyrosine at position 1292 of the Werner protein indicate that the subject has a risk of developing colon cancer.

16. The method of claim 1, wherein step (a) further comprises isolating a gene encoding the TERT gene from the peripheral blood lymphocytes and determining whether the gene encodes an arginine at position 198 of the Telomerase Reverse Transcriptase protein, wherein the presence of elevated levels of gamma-H2AX foci and chromosomal genomic instability, and a polymorphism in the TERT gene encoding an arginine at position 198 of the Telomerase Reverse Transcriptase protein indicate that the subject has a risk of developing colon cancer.

17. The method of claim 1, wherein step (a) further comprises isolating a gene encoding the FAAP100 gene from the peripheral blood lymphocytes and determining whether the gene encodes a leucine at position 466 of the Fanconi anemia-associated protein of 100 kD, wherein the presence of elevated levels of gamma-H2AX foci and chromosomal genomic instability, and a polymorphism in the FAAP100 gene encoding a leucine at position 466 of the Fanconi anemia-associated protein of 100 kD indicate that the subject has a risk of developing colon cancer.

18. The method of claim 1, wherein step (a) further comprises isolating a gene encoding the SHPRH gene from the peripheral blood lymphocytes and determining whether the gene encodes a cysteine at position 1184 of the SNF2 histone linker PHD RING finger helicase protein, wherein the presence of elevated levels of gamma-H2AX foci and chromosomal genomic instability, and a polymorphism in the SHPRH gene encoding a cysteine at position 1184 of the SNF2 histone linker PHD RING finger helicase protein indicate that the subject has a risk of developing colon cancer.


SEQUENCE LISTING (30 sequences)

SEQ ID NO: 1 - DNA, 4479 bp, Homo sapiens
atgccaaatgagggaatcccccactcaagtcaaactcaggagcaagactgtttacagagtcaacctgtcagtaataatgaagaaatggcaatcaagcaagaaagtggtggtgatggggaggtggaggagtacctctcctttcgttctgtgggtgacgggctgtccacctctgctgtggggtgcgcatcagcagctccgaggagagggccagccctgctgcacatcgaccgacatcagatccaggcagtagagcctagcgcccaggcccttgagctgcagggtttgggtgtggacgtctatgaccaggacgtgctggaacagggagtgcttcagcaggtggacaatgccatccatgaggccagccgtgcctcccagctcgttgacgtggagaaggagtatcggtcggtcctggatgacctcacgtcatgtacgacatccctaaggcaaatcaataaaattattgaacagcttagccctcaagctgccaccagcagagacatcaacaggaaactagattctgtaaaacgacagaagtattataaggaacaacagctaaaaaagatcactgcaaaacaaaagcatctccaggccatccttggaggagcagaggtgaaaattgaactagatcacgccagtctggaggaggatgcagagccggggccatccagtcttggcagcatgctcatgcctgtccaggagactgcctgggaagagctcatccgcactggccagatgacaccttttggtacccagatccctcagaaacaggagaaaaagcccagaaaaatcatgcttaatgaagcatcaggcttcgaaaagtatttggcagatcaagcaaaactgtcttttgaaaggaagaagcaaggttgtaataaaagagcagctagaaaagctccagccccagtcacgcctccagccccagtgcaaaataaaaacaaaccaaacaagaaagccagagttctgtccaaaaaagaggagcgtttgaaaaagcacatcaagaaactccagaagagggctttgcagttccaggggaaagtgggattgccaaaggcaaggagaccttgggagtcagacatgaggccagaggcagagggagactctgagggtgaagagtctgagtatttccccacagaggaggaggaagaggaggaagatgacgaggtggagggggcagaggcggacctgtctggagatggtactgactatgagctgaagcctctgcccaagggcgggaaacggcagaagaaagtgccagtgcaggagattgatgatgactttttcccaagttctggggaagaagctgaagctgcttctgtaggagaaggaggaggaggaggtcggaaagtgggaagataccgagatgatggagatgaagattattataagcagcggttaaggagatggaataaactgagactgcaggacaaagagaaacgtctgaagctggaggacgattctgaggaaagtgatgctgaatttgacgaaggttttaaagtgccaggttttctgttcaaaaagctttttaagtaccagcagacaggtgttaggtggctgtgggaattgcactgccagcaggcaggaggaattctgggagatgaaatgggattgggcaagaccatccagataattgccttcttggcaggtctgagctacagcaagatcaggactcgtggttcaaattacaggtttgaggggttgggtccaactgtaattgtctgtccaacaacagtgatgcatcagtgggtgaaggaatttcacacgtggtggcctccgttcagagtggcaattctacatgaaaccggttcctatacccacaaaaaggagaaactaattcgagatgttgctcattgtcatggaattttgatcacatcttactcctacattcgattgatgcaggatgacattagcaggtatgactggcactatgtgatcttggacgaaggacacaaaattcgaaatccaaatgctgctgtcacccttgcttgcaaacagtttcgcacccctcatcggatcattctgtctggctcaccgatgcaaaataacctccgagagctgtggtcgctctttgacttcatcttcccgggaaagttaggcacgttgcctgtgtttatggagcagttctccgtccccatcaccatggggggatattcaaatgcttccccagtacaggtcaaaactgcttacaagtgtgcatgtgtcttacgagataccataaatccatacctactgcggagaatgaagtcagatgtcaagatgagcctttctttgccagataaaaatgaacaggtcttattttgccgtcttacagatgagcagcataaagtctaccaaaatttcgttgattccaaagaagtttacaggattctcaatggagagatgcagattttctccggacttatagccctaagaaaaatttgcaaccaccctgatctcttttctggaggtcccaagaatctcaaaggtcttcctgatgatgaactagaagaagatcagtttgggtactggaaacgttctgggaaaatgattgttgttgagtctttgttgaaaatatggcacaagcagggtcagcgagtattgctgttttctcagtcaaggcagatgctggacatacttgaagtattccttagagcccaaaagtatacctatctcaagatggatggtaccactacaatagcttcaagacagccactgattacgagatacaatgaggacacatccatatttgtgtttcttctgaccacgcgggtgggcggcttaggtgtcaacctgacgggggcaaacagagttgtcatctatgacccagactggaacccaagcacggacacgcaggcccgggagcgagcatggagaataggccagaagaagcaagtgactgtgtacaggctcctgactgcgggcaccattgaagaaaagatctaccaccgacaaatcttcaagcagtttttgacaaatagagtgctaaaagacccaaaacaaaggcggtttttcaaatccaatgatctctatgagctatttactctgactagtcctgatgcatcccagagcactgaaacaagtgcaatttttgcaggaactggatcagatgttcagacacccaaatgccatctaaaaagaaggattcaaccagcctttggagcagaccatgatgttccaaaacgcaagaagttccctgcttctaacatatctgtaaatgatgccacatcatctgaagagaaatctgaggctaaaggagctgaagtaaatgcagtaacttctaatcgaagtgatcctttgaaagatgaccctcacatgagtagtaatgtaactagcaatgataggcttggagaagagacaaatgcagtatctggaccagaagagttgtcagtgattagtggaaatggggaatgttcaaattcttcaggaacaggcaaaacttctatgccatctggtgatgaaagcattgatgaaaagttaggtctttcttacaaaagagaaagacccagccaggctcaaacagaagctttttgggagaataaacaaatggaaaataatttttataagcacaagtcaaaaacaaaacatcatagtgtggcagaagaagagaccctggagaaacatctgagaccaaagcaaaagcctaagaactctaagcattgcagagacgccaagtttgaaggaactcgaattccacacctggtgaagaaaaggcgttaccagaagcaagacagtgaaaacaagagtgaggccaaggaacagagcaatgacgattatgttttggaaaagcttttcaaaaaatcagttggcgtgcacagtgtcatgaagcacgatgccatcatggatggagccagcccagattatgtactggtggaggcagaagccaaccgagtggcccaggatgccctgaaagcactgaggctctctcgtcagcggtgtctgggagcagtgtctggtgttcccacctggactggccacagggggatttctggtgcaccagcaggaaaaaagagtagatttggtaagaaaaggaattctaacttctctgtgcagcatccttcatcaacatctccaacagagaagtgccaggatggcatcatgaaaaaggagggaaaagataatgtccctgagcattttagtggaagagcagaagatgcagactcttcatccgggcccctcgcttcctcctcactcttggctaaaatgagagctagaaaccacctgattctgccagagcgtttagaaagtgaaagcgggcacctgcaggaagcttctgccctgctgcccaccacagaacacgatgaccttctggtggagatgagaaacttcatcgctttccaggcccacactgatggccaggccagcaccagggagatactgcaggagtttgaatccaagttatctgcatcacagtcttgtgtcttccgagaactattgagaaatctgtgcactttccatagaacttctggtggtgaaggaatttggaaactcaagccagaatactgc

SEQ ID NO: 2 - DNA, 4479 bp, Homo sapiens
atgccaaatgagggaatcccccactcaagtcaaactcaggagcaagactgtttacagagtcaacctgtcagtaataatgaagaaatggcaatcaagcaagaaagtggtggtgatggggaggtggaggagtacctctcctttcgttctgtgggtgacgggctgtccacctctgctgtggggtgcgcatcagcagctccgaggagagggccagccctgctgcacatcgaccgacatcagatccaggcagtagagcctagcgcccaggcccttgagctgcagggtttgggtgtggacgtctatgaccaggacgtgctggaacagggagtgcttcagcaggtggacaatgccatccatgaggccagccgtgcctcccagctcgttgacgtggagaaggagtatcggtcggtcctggatgacctcacgtcatgtacgacatccctaaggcaaatcaataaaattattgaacagcttagccctcaagctgccaccagcagagacatcaacaggaaactagattctgtaaaacgacagaagtataataaggaacaacagctaaaaaagatcactgcaaaacaaaagcatctccaggccatccttggaggagcagaggtgaaaattgaactagatcacgccagtctggaggaggatgcagagccggggccatccagtcttggcagcatgctcatgcctgtccaggagactgcctgggaagagctcatccgcactggccagatgacaccttttggtacccagatccctcagaaacaggagaaaaagcccagaaaaatcatgcttaatgaagcatcaggcttcgaaaagtatttggcagatcaagcaaaactgtcttttgaaaggaagaagcaaggttgtaataaaagagcagctagaaaagctccagccccagtcacgcctccagccccagtgcaaaataaaaacaaaccaaacaagaaagccagagttctgtccaaaaaagaggagcgtttgaaaaagcacatcaagaaactccagaagagggctttgcagttccaggggaaagtgggattgccaaaggcaaggagaccttgggagtcagacatgaggccagaggcagagggagactctgagggtgaagagtctgagtatttccccacagaggaggaggaagaggaggaagatgacgaggtggagggggcagaggcggacctgtctggagatggtactgactatgagctgaagcctctgcccaagggcgggaaacggcagaagaaagtgccagtgcaggagattgatgatgactttttcccaagttctggggaagaagctgaagctgcttctgtaggagaaggaggaggaggaggtcggaaagtgggaagataccgagatgatggagatgaagattattataagcagcggttaaggagatggaataaactgagactgcaggacaaagagaaacgtctgaagctggaggacgattctgaggaaagtgatgctgaatttgacgaaggttttaaagtgccaggttttctgttcaaaaagctttttaagtaccagcagacaggtgttaggtggctgtgggaattgcactgccagcaggcaggaggaattctgggagatgaaatgggattgggcaagaccatccagataattgccttcttggcaggtctgagctacagcaagatcaggactcgtggttcaaattacaggtttgaggggttgggtccaactgtaattgtctgtccaacaacagtgatgcatcagtgggtgaaggaatttcacacgtggtggcctccgttcagagtggcaattctacatgaaaccggttcctatacccacaaaaaggagaaactaattcgagatgttgctcattgtcatggaattttgatcacatcttactcctacattcgattgatgcaggatgacattagcaggtatgactggcactatgtgatcttggacgaaggacacaaaattcgaaatccaaatgctgctgtcacccttgcttgcaaacagtttcgcacccctcatcggatcattctgtctggctcaccgatgcaaaataacctccgagagctgtggtcgctctttgacttcatcttcccgggaaagttaggcacgttgcctgtgtttatggagcagttctccgtccccatcaccatggggggatattcaaatgcttccccagtacaggtcaaaactgcttacaagtgtgcatgtgtcttacgagataccataaatccatacctactgcggagaatgaagtcagatgtcaagatgagcctttctttgccagataaaaatgaacaggtcttattttgccgtcttacagatgagcagcataaagtctaccaaaatttcgttgattccaaagaagtttacaggattctcaatggagagatgcagattttctccggacttatagccctaagaaaaatttgcaaccaccctgatctcttttctggaggtcccaagaatctcaaaggtcttcctgatgatgaactagaagaagatcagtttgggtactggaaacgttctgggaaaatgattgttgttgagtctttgttgaaaatatggcacaagcagggtcagcgagtattgctgttttctcagtcaaggcagatgctggacatacttgaagtattccttagagcccaaaagtatacctatctcaagatggatggtaccactacaatagcttcaagacagccactgattacgagatacaatgaggacacatccatatttgtgtttcttctgaccacgcgggtgggcggcttaggtgtcaacctgacgggggcaaacagagttgtcatctatgacccagactggaacccaagcacggacacgcaggcccgggagcgagcatggagaataggccagaagaagcaagtgactgtgtacaggctcctgactgcgggcaccattgaagaaaagatctaccaccgacaaatcttcaagcagtttttgacaaatagagtgctaaaagacccaaaacaaaggcggtttttcaaatccaatgatctctatgagctatttactctgactagtcctgatgcatcccagagcactgaaacaagtgcaatttttgcaggaactggatcagatgttcagacacccaaatgccatctaaaaagaaggattcaaccagcctttggagcagaccatgatgttccaaaacgcaagaagttccctgcttctaacatatctgtaaatgatgccacatcatctgaagagaaatctgaggctaaaggagctgaagtaaatgcagtaacttctaatcgaagtgatcctttgaaagatgaccctcacatgagtagtaatgtaactagcaatgataggcttggagaagagacaaatgcagtatctggaccagaagagttgtcagtgattagtggaaatggggaatgttcaaattcttcaggaacaggcaaaacttctatgccatctggtgatgaaagcattgatgaaaagttaggtctttcttacaaaagagaaagacccagccaggctcaaacagaagctttttgggagaataaacaaatggaaaataatttttataagcacaagtcaaaaacaaaacatcatagtgtggcagaagaagagaccctggagaaacatctgagaccaaagcaaaagcctaagaactctaagcattgcagagacgccaagtttgaaggaactcgaattccacacctggtgaagaaaaggcgttaccagaagcaagacagtgaaaacaagagtgaggccaaggaacagagcaatgacgattatgttttggaaaagcttttcaaaaaatcagttggcgtgcacagtgtcatgaagcacgatgccatcatggatggagccagcccagattatgtactggtggaggcagaagccaaccgagtggcccaggatgccctgaaagcactgaggctctctcgtcagcggtgtctgggagcagtgtctggtgttcccacctggactggccacagggggatttctggtgcaccagcaggaaaaaagagtagatttggtaagaaaaggaattctaacttctctgtgcagcatccttcatcaacatctccaacagagaagtgccaggatggcatcatgaaaaaggagggaaaagataatgtccctgagcattttagtggaagagcagaagatgcagactcttcatccgggcccctcgcttcctcctcactcttggctaaaatgagagctagaaaccacctgattctgccagagcgtttagaaagtgaaagcgggcacctgcaggaagcttctgccctgctgcccaccacagaacacgatgaccttctggtggagatgagaaacttcatcgctttccaggcccacactgatggccaggccagcaccagggagatactgcaggagtttgaatccaagttatctgcatcacagtcttgtgtcttccgagaactattgagaaatctgtgcactttccatagaacttctggtggtgaaggaatttggaaactcaagccagaatactgc

SEQ ID NO: 3 - DNA, 7006 bp, Homo sapiens
agcagaagtcggagtcgctgttgggggcggtgtctatggttgagctgagggcgcaggcgccacggcccgtcgagctgggttccaaggcggctggcggcggtagcgtctctgtttccttgtgggcgctcgcgcggccctgggtagtctgtagagaatgccaaatgagggaatcccccactcaagtcaaactcaggagcaagactgtttacagagtcaacctgtcagtaataatgaagaaatggcaatcaagcaagaaagtggtggtgatggggaggtggaggagtacctctcctttcgttctgtgggtgacgggctgtccacctctgctgtggggtgcgcatcagcagctccgaggagagggccagccctgctgcacatcgaccgacatcagatccaggcagtagagcctagcgcccaggcccttgagctgcagggtttgggtgtggacgtctatgaccaggacgtgctggaacagggagtgcttcagcaggtggacaatgccatccatgaggccagccgtgcctcccagctcgttgacgtggagaaggagtatcggtcggtcctggatgacctcacgtcatgtacgacatccctaaggcaaatcaataaaattattgaacagcttagccctcaagctgccaccagcagagacatcaacaggaaactagattctgtaaaacgacagaagtataataaggaacaacagctaaaaaagatcactgcaaaacaaaagcatctccaggccatccttggaggagcagaggtgaaaattgaactagatcacgccagtctggaggaggatgcagagccggggccatccagtcttggcagcatgctcatgcctgtccaggagactgcctgggaagagctcatccgcactggccagatgacaccttttggtacccagatccctcagaaacaggagaaaaagcccagaaaaatcatgcttaatgaagcatcaggcttcgaaaagtatttggcagatcaagcaaaactgtcttttgaaaggaagaagcaaggttgtaataaaagagcagctagaaaagctccagccccagtcacgcctccagccccagtgcaaaataaaaacaaaccaaacaagaaagccagagttctgtccaaaaaagaggagcgtttgaaaaagcacatcaagaaactccagaagagggctttgcagttccaggggaaagtgggattgccaaaggcaaggagaccttgggagtcagacatgaggccagaggcagagggagactctgagggtgaagagtctgagtatttccccacagaggaggaggaagaggaggaagatgacgaggtggagggggcagaggcggacctgtctggagatggtactgactatgagctgaagcctctgcccaagggcgggaaacggcagaagaaagtgccagtgcaggagattgatgatgactttttcccaagttctggggaagaagctgaagctgcttctgtaggagaaggaggaggaggaggtcggaaagtgggaagataccgagatgatggagatgaagattattataagcagcggttaaggagatggaataaactgagactgcaggacaaagagaaacgtctgaagctggaggacgattctgaggaaagtgatgctgaatttgacgaaggttttaaagtgccaggttttctgttcaaaaagctttttaagtaccagcagacaggtgttaggtggctgtgggaattgcactgccagcaggcaggaggaattctgggagatgaaatgggattgggcaagaccatccagataattgccttcttggcaggtctgagctacagcaagatcaggactcgtggttcaaattacaggtttgaggggttgggtccaactgtaattgtctgtccaacaacagtgatgcatcagtgggtgaaggaatttcacacgtggtggcctccgttcagagtggcaattctacatgaaaccggttcctatacccacaaaaaggagaaactaattcgagatgttgctcattgtcatggaattttgatcacatcttactcctacattcgattgatgcaggatgacattagcaggtatgactggcactatgtgatcttggacgaaggacacaaaattcgaaatccaaatgctgctgtcacccttgcttgcaaacagtttcgcacccctcatcggatcattctgtctggctcaccgatgcaaaataacctccgagagctgtggtcgctctttgacttcatcttcccgggaaagttaggcacgttgcctgtgtttatggagcagttctccgtccccatcaccatggggggatattcaaatgcttccccagtacaggtcaaaactgcttacaagtgtgcatgtgtcttacgagataccataaatccatacctactgcggagaatgaagtcagatgtcaagatgagcctttctttgccagataaaaatgaacaggtcttattttgccgtcttacagatgagcagcataaagtctaccaaaatttcgttgattccaaagaagtttacaggattctcaatggagagatgcagattttctccggacttatagccctaagaaaaatttgcaaccaccctgatctcttttctggaggtcccaagaatctcaaaggtcttcctgatgatgaactagaagaagatcagtttgggtactggaaacgttctgggaaaatgattgttgttgagtctttgttgaaaatatggcacaagcagggtcagcgagtattgctgttttctcagtcaaggcagatgctggacatacttgaagtattccttagagcccaaaagtatacctatctcaagatggatggtaccactacaatagcttcaagacagccactgattacgagatacaatgaggacacatccatatttgtgtttcttctgaccacgcgggtgggcggcttaggtgtcaacctgacgggggcaaacagagttgtcatctatgacccagactggaacccaagcacggacacgcaggcccgggagcgagcatggagaataggccagaagaagcaagtgactgtgtacaggctcctgactgcgggcaccattgaagaaaagatctaccaccgacaaatcttcaagcagtttttgacaaatagagtgctaaaagacccaaaacaaaggcggtttttcaaatccaatgatctctatgagctatttactctgactagtcctgatgcatcccagagcactgaaacaagtgcaatttttgcaggaactggatcagatgttcagacacccaaatgccatctaaaaagaaggattcaaccagcctttggagcagaccatgatgttccaaaacgcaagaagttccctgcttctaacatatctgtaaatgatgccacatcatctgaagagaaatctgaggctaaaggagctgaagtaaatgcagtaacttctaatcgaagtgatcctttgaaagatgaccctcacatgagtagtaatgtaactagcaatgataggcttggagaagagacaaatgcagtatctggaccagaagagttgtcagtgattagtggaaatggggaatgttcaaattcttcaggaacaggcaaaacttctatgccatctggtgatgaaagcattgatgaaaagttaggtctttcttacaaaagagaaagacccagccaggctcaaacagaagctttttgggagaataaacaaatggaaaataatttttataagcacaagtcaaaaacaaaacatcatagtgtggcagaagaagagaccctggagaaacatctgagaccaaagcaaaagcctaagaactctaagcattgcagagacgccaagtttgaaggaactcgaattccacacctggtgaagaaaaggcgttaccagaagcaagacagtgaaaacaagagtgaggccaaggaacagagcaatgacgattatgttttggaaaagcttttcaaaaaatcagttggcgtgcacagtgtcatgaagcacgatgccatcatggatggagccagcccagattatgtactggtggaggcagaagccaaccgagtggcccaggatgccctgaaagcactgaggctctctcgtcagcggtgtctgggagcagtgtctggtgttcccacctggactggccacagggggatttctggtgcaccagcaggaaaaaagagtagatttggtaagaaaaggaattctaacttctctgtgcagcatccttcatcaacatctccaacagagaagtgccaggatggcatcatgaaaaaggagggaaaagataatgtccctgagcattttagtggaagagcagaagatgcagactcttcatccgggcccctcgcttcctcctcactcttggctaaaatgagagctagaaaccacctgattctgccagagcgtttagaaagtgaaagcgggcacctgcaggaagcttctgccctgctgcccaccacagaacacgatgaccttctggtggagatgagaaacttcatcgctttccaggcccacactgatggccaggccagcaccagggagatactgcaggagtttgaatccaagttatctgcatcacagtcttgtgtcttccgagaactattgagaaatctgtgcactttccatagaacttctggtggtgaaggaatttggaaactcaagccagaatactgctaaacaacattgcttcctaaactttcaagtccctttttctaacgggcatttctgattattaatttattattaataatcatgtttgtcaatggaagttggctgcacttgatgtttgtttgcatgatgtctacctcagaattaaaactttaaggaagaagaaactcttctctgaaagttaaaagttttaataatgctagctaaaggagaaaatacttggattgatttttttttttttggcaatctaattatattgtaaatcaggtacctaacagttactccttggagcacatttgttcctttacccaaaagatgctgtcagggagcacagttagaagtttgcagaacagaaatctcaatatttttttttattggtgctaaaaacaggtcttacattcagtcagacctgttcaataagttcatcaatatctgataacagcattattttgatgcttaaactttaaacatttatatttaccatttgccacccacaaaggtcaggtttgttatttgttgtttgataattatattaattttcttggaaagatcctcttttcaaggtactggtaaattggtgagtatttttattagtaaagcatgaaatagtatggtaataaatgataagacatgtatttgtggaaagctgtagggtattcagtttaccctggctttcctttaagcagagggcatctttttctctcctacagtcacaaaatgtgttatcattaaaaaaaatcaaattaaagccaaaagtaggtacataaaaaccacacacatgcatgcacacaaacatcactgcagcccacagcagacccagccgttgttaccatgaagtgacaccactccaggcctctcttgtctgcaggctggcaggctgtcttctctccagttgccttcgtcttgcgcctgcctttgcattccttgcgacgggctttcttgtttctgcggtttggattccagccaaggctgtttgtatctcactactgtttatgtgtttgtggttctgtgatggtgttgctttgatcctcagtttattttcttacccatgtttttcttgtttccttctcaggatgattttatcatctcatctttgaagtgttgttttccgaaattcatcgtattcctgaaatttcttcttagctgtcttagtgcagtttgtttcttggatttgtattctctggcatgctcttttcctctctctcatttttctgtagtatgcctgccctcctaccctgctatttctttacatctctctcatgcttaacatggatagctgtgtccagatcttctgtctgctcatccatgtgactcagagaggagggttctgggcaggggggccttgccggactgcatgagaggacatgagttttgctttctctgctctaatattttgcttaagccaagaatccttttcttagagatgttctatatgattcctgtcaggattttctagttttttttggattatagcttgttcatttcttttgtttttagtttggtttatatataatgagggaagaagatgattacattatttttgtcactttgccatcattgtttagaagtcatagaaagaatttttaaataggccaataagtcttaaacttgagtacttggcttagaagaaagtcaaaactccttcctttttgactaagtggtttgtttctggggagctcttaatttctatttttataatcattagcctataaggaaattgtgtcttccttgttctcagggtgatctgctgaccttgttcactcatgaagcatttgggtatcatacttatagtgtctgaaacataaactgtattgagctagacaaggtatagcctcctcttcaagtagcaaatactatcaaaagctataatgcagtaggagcaaggtggtccttgttccagtttttgtctcagttctgctgctgatgtaccatgatcttgggaaggtggtgtctcagtgtggagatctgacacattgttaccgtgcctcctggctggagggacttggagaacaatgcagttaagtagaatggttttaacaatacagagaaatttattcatttagataaaaatctgatttttagaactttaaaagctttgtacagtgtaaatagatttaatgtatttaacatgctttatcagcacaaataaaggattttaaaattttgtcaaaaaattaaatgttaatactatcaccattaaaaatgttcaagcaatagtctgcctccccacccccacaccatcttgcacctgttccacagctaagtacagccctaggtttggtgtgtattctccatgcatttagagaatcacatgacacagactgctgctataatgtcattttcccattcttcctttactaataaaatttttgagtttta

SEQ ID NO: 4 - protein, 1493 aa, Homo sapiens
MPNEGIPHSSQTQEQDCLQSQPVSNNEEMAIKQESGGDGEVEEYLSFRSVGDGLSTSAVGCASAAPRRGPALLHIDRHQIQAVEPSAQALELQGLGVDVYDQDVLEQGVLQQVDNAIHEASRASQLVDVEKEYRSVLDDLTSCTTSLRQINKIIEQLSPQAATSRDINRKLDSVKRQKYYKEQQLKKITAKQKHLQAILGGAEVKIELDHASLEEDAEPGPSSLGSMLMPVQETAWEELIRTGQMTPFGTQIPQKQEKKPRKIMLNEASGFEKYLADQAKLSFERKKQGCNKRAARKAPAPVTPPAPVQNKNKPNKKARVLSKKEERLKKHIKKLQKRALQFQGKVGLPKARRPWESDMRPEAEGDSEGEESEYFPTEEEEEEEDDEVEGAEADLSGDGTDYELKPLPKGGKRQKKVPVQEIDDDFFPSSGEEAEAASVGEGGGGGRKVGRYRDDGDEDYYKQRLRRWNKLRLQDKEKRLKLEDDSEESDAEFDEGFKVPGFLFKKLFKYQQTGVRWLWELHCQQAGGILGDEMGLGKTIQIIAFLAGLSYSKIRTRGSNYRFEGLGPTVIVCPTTVMHQWVKEFHTWWPPFRVAILHETGSYTHKKEKLIRDVAHCHGILITSYSYIRLMQDDISRYDWHYVILDEGHKIRNPNAAVTLACKQFRTPHRIILSGSPMQNNLRELWSLFDFIFPGKLGTLPVFMEQFSVPITMGGYSNASPVQVKTAYKCACVLRDTINPYLLRRMKSDVKMSLSLPDKNEQVLFCRLTDEQHKVYQNFVDSKEVYRILNGEMQIFSGLIALRKICNHPDLFSGGPKNLKGLPDDELEEDQFGYWKRSGKMIVVESLLKIWHKQGQRVLLFSQSRQMLDILEVFLRAQKYTYLKMDGTTTIASRQPLITRYNEDTSIFVFLLTTRVGGLGVNLTGANRVVIYDPDWNPSTDTQARERAWRIGQKKQVTVYRLLTAGTIEEKIYHRQIFKQFLTNRVLKDPKQRRFFKSNDLYELFTLTSPDASQSTETSAIFAGTGSDVQTPKCHLKRRIQPAFGADHDVPKRKKFPASNISVNDATSSEEKSEAKGAEVNAVTSNRSDPLKDDPHMSSNVTSNDRLGEETNAVSGPEELSVISGNGECSNSSGTGKTSMPSGDESIDEKLGLSYKRERPSQAQTEAFWENKQMENNFYKHKSKTKHHSVAEEETLEKHLRPKQKPKNSKHCRDAKFEGTRIPHLVKKRRYQKQDSENKSEAKEQSNDDYVLEKLFKKSVGVHSVMKHDAIMDGASPDYVLVEAEANRVAQDALKALRLSRQRCLGAVSGVPTWTGHRGISGAPAGKKSRFGKKRNSNFSVQHPSSTSPTEKCQDGIMKKEGKDNVPEHFSGRAEDADSSSGPLASSSLLAKMRARNHLILPERLESESGHLQEASALLPTTEHDDLLVEMRNFIAFQAHTDGQASTREILQEFESKLSASQSCVFRELLRNLCTFHRTSGGEGIWKLKPEYC

SEQ ID NO: 5 - protein, 1493 aa, Homo sapiens
MPNEGIPHSSQTQEQDCLQSQPVSNNEEMAIKQESGGDGEVEEYLSFRSVGDGLSTSAVGCASAAPRRGPALLHIDRHQIQAVEPSAQALELQGLGVDVYDQDVLEQGVLQQVDNAIHEASRASQLVDVEKEYRSVLDDLTSCTTSLRQINKIIEQLSPQAATSRDINRKLDSVKRQKYNKEQQLKKITAKQKHLQAILGGAEVKIELDHASLEEDAEPGPSSLGSMLMPVQETAWEELIRTGQMTPFGTQIPQKQEKKPRKIMLNEASGFEKYLADQAKLSFERKKQGCNKRAARKAPAPVTPPAPVQNKNKPNKKARVLSKKEERLKKHIKKLQKRALQFQGKVGLPKARRPWESDMRPEAEGDSEGEESEYFPTEEEEEEEDDEVEGAEADLSGDGTDYELKPLPKGGKRQKKVPVQEIDDDFFPSSGEEAEAASVGEGGGGGRKVGRYRDDGDEDYYKQRLRRWNKLRLQDKEKRLKLEDDSEESDAEFDEGFKVPGFLFKKLFKYQQTGVRWLWELHCQQAGGILGDEMGLGKTIQIIAFLAGLSYSKIRTRGSNYRFEGLGPTVIVCPTTVMHQWVKEFHTWWPPFRVAILHETGSYTHKKEKLIRDVAHCHGILITSYSYIRLMQDDISRYDWHYVILDEGHKIRNPNAAVTLACKQFRTPHRIILSGSPMQNNLRELWSLFDFIFPGKLGTLPVFMEQFSVPITMGGYSNASPVQVKTAYKCACVLRDTINPYLLRRMKSDVKMSLSLPDKNEQVLFCRLTDEQHKVYQNFVDSKEVYRILNGEMQIFSGLIALRKICNHPDLFSGGPKNLKGLPDDELEEDQFGYWKRSGKMIVVESLLKIWHKQGQRVLLFSQSRQMLDILEVFLRAQKYTYLKMDGTTTIASRQPLITRYNEDTSIFVFLLTTRVGGLGVNLTGANRVVIYDPDWNPSTDTQARERAWRIGQKKQVTVYRLLTAGTIEEKIYHRQIFKQFLTNRVLKDPKQRRFFKSNDLYELFTLTSPDASQSTETSAIFAGTGSDVQTPKCHLKRRIQPAFGADHDVPKRKKFPASNISVNDATSSEEKSEAKGAEVNAVTSNRSDPLKDDPHMSSNVTSNDRLGEETNAVSGPEELSVISGNGECSNSSGTGKTSMPSGDESIDEKLGLSYKRERPSQAQTEAFWENKQMENNFYKHKSKTKHHSVAEEETLEKHLRPKQKPKNSKHCRDAKFEGTRIPHLVKKRRYQKQDSENKSEAKEQSNDDYVLEKLFKKSVGVHSVMKHDAIMDGASPDYVLVEAEANRVAQDALKALRLSRQRCLGAVSGVPTWTGHRGISGAPAGKKSRFGKKRNSNFSVQHPSSTSPTEKCQDGIMKKEGKDNVPEHFSGRAEDADSSSGPLASSSLLAKMRARNHLILPERLESESGHLQEASALLPTTEHDDLLVEMRNFIAFQAHTDGQASTREILQEFESKLSASQSCVFRELLRNLCTFHRTSGGEGIWKLKPEYC

SEQ ID NO: 6 - DNA, 4296 bp, Homo sapiens
atgagtgaaaaaaaattggaaacaactgcacagcagcggaaatgtcctgaatggatgaatgtgcagaataaaagatgtgctgtagaagaaagaaaggcatgtgttcggaagagtgtttttgaagatgacctccccttcttagaattcactggatccattgtgtatagttacgatgctagtgattgctctttcctgtcagaagatattagcatgagtctatcagatggggatgtggtgggatttgacatggagtggccaccattatacaatagagggaaacttggcaaagttgcactaattcagttgtgtgtttctgagagcaaatgttacttgttccacgtttcttccatgtcagtttttccccagggattaaaaatgttgcttgaaaataaagcagttaaaaaggcaggtgtaggaattgaaggagatcagtggaaacttctacgtgactttgatatcaaattgaagaattttgtggagttgacagatgttgccaataaaaagctgaaatgcacagagacctggagccttaacagtctggttaaacacctcttaggtaaacagctcctgaaagacaagtctatccgctgtagcaattggagtaaatttcctctcactgaggaccagaaactgtatgcagccactgatgcttatgctggttttattatttaccgaaatttagagattttggatgatactgtgcaaaggtttgctataaataaagaggaagaaatcctacttagcgacatgaacaaacagttgacttcaatctctgaggaagtgatggatctggctaagcatcttcctcatgctttcagtaaattggaaaacccacggagggtttctatcttactaaaggatatttcagaaaatctatattcactgaggaggatgataattgggtctactaacattgagactgaactgaggcccagcaataatttaaacttattatcctttgaagattcaactactgggggagtacaacagaaacaaattagagaacatgaagttttaattcacgttgaagatgaaacatgggacccaacacttgatcatttagctaaacatgatggagaagatgtacttggaaataaagtggaacgaaaagaagatggatttgaagatggagtagaagacaacaaattgaaagagaatatggaaagagcttgtttgatgtcgttagatattacagaacatgaactccaaattttggaacagcagtctcaggaagaatatcttagtgatattgcttataaatctactgagcatttatctcccaatgataatgaaaacgatacgtcctatgtaattgagagtgatgaagatttagaaatggagatgcttaagcatttatctcccaatgataatgaaaacgatacgtcctatgtaattgagagtgatgaagatttagaaatggagatgcttaagtctttagaaaacctcaatagtggcacggtagaaccaactcattctaaatgcttaaaaatggaaagaaatctgggtcttcctactaaagaagaagaagaagatgatgaaaatgaagctaatgaaggggaagaagatgatgataaggactttttgtggccagcacccaatgaagagcaagttacttgcctcaagatgtactttggccattccagttttaaaccagttcagtggaaagtgattcattcagtattagaagaaagaagagataatgttgctgtcatggcaactggatatggaaagagtttgtgcttccagtatccacctgtttatgtaggcaagattggccttgttatctctccccttatttctctgatggaagaccaagtgctacagcttaaaatgtccaacatcccagcttgcttccttggatcagcacagtcagaaaatgttctaacagatattaaattaggtaaataccggattgtatacgtaactccagaatactgttcaggtaacatgggcctgctccagcaacttgaggctgatattggtatcacgctcattgctgtggatgaggctcactgtatttctgagtgggggcatgattttagggattcattcaggaagttgggctccctaaagacagcactgccaatggttccaatcgttgcacttactgctattgcaagttcttcaatccgggaagacattgtacgttgcttaaatctgagaaatcctcagatcacctgtactggttttgatcgaccaaacctgtatttagaagttaggcgaaaaacagggaatatccttcaggatctgcagccatttcttgtcaaaacaagttcccactgggaatttgaaggtccaacaatcatctactgtccttctagaaaaatgacacaacaagttacaggtgaacttaggaaactgaatctatcctgtggaacataccatgcgggcatgagttttagcacaaggaaagacattcatcataggtttgtaagagatgaaattcagtgtgtcatagctaccatagcttttggaatgggcattaataaagctgacattcgccaagtcattcattacggtgctcctaaggacatggaatcatattatcaggagattggtagagctggtcgtgatggacttcaaagttcttgtcacgtcctctgggctcctgcagacattaacttaaataggcaccttcttactgagatacgtaatgagaagtttcgattatacaaattaaagatgatggcaaagatggaaaaatatcttcattctagcagatgtaggagacaaatcatcttgtctcattttgaggacaaacaagtacaaaaagcctccttgggaattatgggaactgaaaaatgctgtgataattgcaggtccagattggatcattgctattccatggatgactcagaggatacatcctgggactttggtccacaagcatttaagcttttgtctgctgtggacatcttaggcgaaaaatttggaattgggcttccaattttatttctccgaggatctaattctcagcgtcttgccgatcaatatcgcaggcacagtttatttggcactggcaaggatcaaacagagagttggtggaaggctttttcccgtcagctgatcactgagggattcttggtagaagtttctcggtataacaaatttatgaagatttgcgcccttacgaaaaagggtagaaattggcttcataaagctaatacagaatctcagagcctcatccttcaagctaatgaagaattgtgtccaaagaagttgcttctgcctagttcgaaaactgtatcttcgggcaccaaagagcattgttataatcaagtaccagttgaattaagtacagagaagaagtctaacttggagaagttatattcttataaaccatgtgataagatttcttctgggagtaacatttctaaaaaaagtatcatggtacagtcaccagaaaaagcttacagttcctcacagcctgttatttcggcacaagagcaggagactcagattgtgttatatggcaaattggtagaagctaggcagaaacatgccaataaaatggatgttcccccagctattctggcaacaaacaagatactggtggatatggccaaaatgagaccaactacggttgaaaacgtaaaaaggattgatggtgtttctgaaggcaaagctgccatgttggcccctctgttggaagtcatcaaacatttctgccaaacaaatagtgttcagacagacctcttttcaagtacaaaacctcaagaagaacagaagacgagtctggtagcaaaaaataaaatatgcacactttcacagtctatggccatcacatactctttattccaagaaaagaagatgcctttgaagagcatagctgagagcaggattctgcctctcatgacaattggcatgcacttatcccaagcggtgaaagctggctgcccccttgatttggagcgagcaggcctgactccagaggttcagaagattattgctgatgttatccgaaaccctcccgtcaactcagatatgagtaaaattagcctaatcagaatgttagttcctgaaaacattgacacgtaccttatccacatggcaattgagatccttaaacatggtcctgacagcggacttcaaccttcatgtgatgtcaacaaaaggagatgttttcccggttctgaagagatctgttcaagttctaagagaagcaaggaagaagtaggcatcaatactgagacttcatctgcagagagaaagagacgattacctgtgtggtttgccaaaggaagtgataccagcaagaaattaatggacaaaacgaaaaggggaggtctttttagt

SEQ ID NO: 7 - DNA, 4296 bp, Homo sapiens
atgagtgaaaaaaaattggaaacaactgcacagcagcggaaatgtcctgaatggatgaatgtgcagaataaaagatgtgctgtagaagaaagaaaggcatgtgttcggaagagtgtttttgaagatgacctccccttcttagaattcactggatccattgtgtatagttacgatgctagtgattgctctttcctgtcagaagatattagcatgagtctatcagatggggatgtggtgggatttgacatggagtggccaccattatacaatagagggaaacttggcaaagttgcactaattcagttgtgtgtttctgagagcaaatgttacttgttccacgtttcttccatgtcagtttttccccagggattaaaaatgttgcttgaaaataaagcagttaaaaaggcaggtgtaggaattgaaggagatcagtggaaacttctacgtgactttgatatcaaattgaagaattttgtggagttgacagatgttgccaataaaaagctgaaatgcacagagacctggagccttaacagtctggttaaacacctcttaggtaaacagctcctgaaagacaagtctatccgctgtagcaattggagtaaatttcctctcactgaggaccagaaactgtatgcagccactgatgcttatgctggttttattatttaccgaaatttagagattttggatgatactgtgcaaaggtttgctataaataaagaggaagaaatcctacttagcgacatgaacaaacagttgacttcaatctctgaggaagtgatggatctggctaagcatcttcctcatgctttcagtaaattggaaaacccacggagggtttctatcttactaaaggatatttcagaaaatctatattcactgaggaggatgataattgggtctactaacattgagactgaactgaggcccagcaataatttaaacttattatcctttgaagattcaactactgggggagtacaacagaaacaaattagagaacatgaagttttaattcacgttgaagatgaaacatgggacccaacacttgatcatttagctaaacatgatggagaagatgtacttggaaataaagtggaacgaaaagaagatggatttgaagatggagtagaagacaacaaattgaaagagaatatggaaagagcttgtttgatgtcgttagatattacagaacatgaactccaaattttggaacagcagtctcaggaagaatatcttagtgatattgcttataaatctactgagcatttatctcccaatgataatgaaaacgatacgtcctatgtaattgagagtgatgaagatttagaaatggagatgcttaagcatttatctcccaatgataatgaaaacgatacgtcctatgtaattgagagtgatgaagatttagaaatggagatgcttaagtctttagaaaacctcaatagtggcacggtagaaccaactcattctaaatgcttaaaaatggaaagaaatctgggtcttcctactaaagaagaagaagaagatgatgaaaatgaagctaatgaaggggaagaagatgatgataaggactttttgtggccagcacccaatgaagagcaagttacttgcctcaagatgtactttggccattccagttttaaaccagttcagtggaaagtgattcattcagtattagaagaaagaagagataatgttgctgtcatggcaactggatatggaaagagtttgtgcttccagtatccacctgtttatgtaggcaagattggccttgttatctctccccttatttctctgatggaagaccaagtgctacagcttaaaatgtccaacatcccagcttgcttccttggatcagcacagtcagaaaatgttctaacagatattaaattaggtaaataccggattgtatacgtaactccagaatactgttcaggtaacatgggcctgctccagcaacttgaggctgatattggtatcacgctcattgctgtggatgaggctcactgtatttctgagtgggggcatgattttagggattcattcaggaagttgggctccctaaagacagcactgccaatggttccaatcgttgcacttactgctactgcaagttcttcaatccgggaagacattgtacgttgcttaaatctgagaaatcctcagatcacctgtactggttttgatcgaccaaacctgtatttagaagttaggcgaaaaacagggaatatccttcaggatctgcagccatttcttgtcaaaacaagttcccactgggaatttgaaggtccaacaatcatctactgtccttctagaaaaatgacacaacaagttacaggtgaacttaggaaactgaatctatcctgtggaacataccatgcgggcatgagttttagcacaaggaaagacattcatcataggtttgtaagagatgaaattcagtgtgtcatagctaccatagcttttggaatgggcattaataaagctgacattcgccaagtcattcattacggtgctcctaaggacatggaatcatattatcaggagattggtagagctggtcgtgatggacttcaaagttcttgtcacgtcctctgggctcctgcagacattaacttaaataggcaccttcttactgagatacgtaatgagaagtttcgattatacaaattaaagatgatggcaaagatggaaaaatatcttcattctagcagatgtaggagacaaatcatcttgtctcattttgaggacaaacaagtacaaaaagcctccttgggaattatgggaactgaaaaatgctgtgataattgcaggtccagattggatcattgctattccatggatgactcagaggatacatcctgggactttggtccacaagcatttaagcttttgtctgctgtggacatcttaggcgaaaaatttggaattgggcttccaattttatttctccgaggatctaattctcagcgtcttgccgatcaatatcgcaggcacagtttatttggcactggcaaggatcaaacagagagttggtggaaggctttttcccgtcagctgatcactgagggattcttggtagaagtttctcggtataacaaatttatgaagatttgcgcccttacgaaaaagggtagaaattggcttcataaagctaatacagaatctcagagcctcatccttcaagctaatgaagaattgtgtccaaagaagttgcttctgcctagttcgaaaactgtatcttcgggcaccaaagagcattgttataatcaagtaccagttgaattaagtacagagaagaagtctaacttggagaagttatattcttataaaccatgtgataagatttcttctgggagtaacatttctaaaaaaagtatcatggtacagtcaccagaaaaagcttacagttcctcacagcctgttatttcggcacaagagcaggagactcagattgtgttatatggcaaattggtagaagctaggcagaaacatgccaataaaatggatgttcccccagctattctggcaacaaacaagatactggtggatatggccaaaatgagaccaactacggttgaaaacgtaaaaaggattgatggtgtttctgaaggcaaagctgccatgttggcccctctgttggaagtcatcaaacatttctgccaaacaaatagtgttcagacagacctcttttcaagtacaaaacctcaagaagaacagaagacgagtctggtagcaaaaaataaaatatgcacactttcacagtctatggccatcacatactctttattccaagaaaagaagatgcctttgaagagcatagctgagagcaggattctgcctctcatgacaattggcatgcacttatcccaagcggtgaaagctggctgcccccttgatttggagcgagcaggcctgactccagaggttcagaagattattgctgatgttatccgaaaccctcccgtcaactcagatatgagtaaaattagcctaatcagaatgttagttcctgaaaacattgacacgtaccttatccacatggcaattgagatccttaaacatggtcctgacagcggacttcaaccttcatgtgatgtcaacaaaaggagatgttttcccggttctgaagagatctgttcaagttctaagagaagcaaggaagaagtaggcatcaatactgagacttcatctgcagagagaaagagacgattacctgtgtggtttgccaaaggaagtgataccagcaagaaattaatggacaaaacgaaaaggggaggtctttttagt

SEQ ID NO: 8 - DNA, 5765 bp, Homo sapiens
cagccgcccctcctgcggccgctgcgggggccgccgcctgacttcggacaccggccccgcacccgccaggaggggagggaaggggaggcggggagagcgacggcggggggcgggcggtggaccccgcctcccccggcacagcctgctgaggggaagagggggtctccgctcttcctcagtgcactctctgactgaagcccggcgcgtggggtgcagcgggagtgcgaggggactggacaggtgggaagatgggaatgaggaccgggcggcgggaatgttctcacttctccggattccaccgggatgcaggactctagctgcccagccgcacctgcgaagagactacacttcccgaggtgctcagcggcagcgagggcctccacgcatgcgcaccgcggcgcgctgggcggggctggatgggctgtggtgggagggttgcagcgccgcgagaaaggcgagccgggccgggggcggggaaaggggtggggcaggaacgggggcggggacggcgctggaggggcgggtcgggtaggtctcccggagctgatgtgtactgtgtgcgccggggaggcgccggcttgtactcggcagcgcgggaataaagtttgctgatttggtgtctagcctggatgcctgggttgcaggccctgcttgtggtggcgctccacagtcatccggctgaagaagacctgttggactggatcttctcgggttttctttcagatattgttttgtatttacccatgaagacattgttttttggactctgcaaataggacatttcaaagatgagtgaaaaaaaattggaaacaactgcacagcagcggaaatgtcctgaatggatgaatgtgcagaataaaagatgtgctgtagaagaaagaaaggcatgtgttcggaagagtgtttttgaagatgacctccccttcttagaattcactggatccattgtgtatagttacgatgctagtgattgctctttcctgtcagaagatattagcatgagtctatcagatggggatgtggtgggatttgacatggagtggccaccattatacaatagagggaaacttggcaaagttgcactaattcagttgtgtgtttctgagagcaaatgttacttgttccacgtttcttccatgtcagtttttccccagggattaaaaatgttgcttgaaaataaagcagttaaaaaggcaggtgtaggaattgaaggagatcagtggaaacttctacgtgactttgatatcaaattgaagaattttgtggagttgacagatgttgccaataaaaagctgaaatgcacagagacctggagccttaacagtctggttaaacacctcttaggtaaacagctcctgaaagacaagtctatccgctgtagcaattggagtaaatttcctctcactgaggaccagaaactgtatgcagccactgatgcttatgctggttttattatttaccgaaatttagagattttggatgatactgtgcaaaggtttgctataaataaagaggaagaaatcctacttagcgacatgaacaaacagttgacttcaatctctgaggaagtgatggatctggctaagcatcttcctcatgctttcagtaaattggaaaacccacggagggtttctatcttactaaaggatatttcagaaaatctatattcactgaggaggatgataattgggtctactaacattgagactgaactgaggcccagcaataatttaaacttattatcctttgaagattcaactactgggggagtacaacagaaacaaattagagaacatgaagttttaattcacgttgaagatgaaacatgggacccaacacttgatcatttagctaaacatgatggagaagatgtacttggaaataaagtggaacgaaaagaagatggatttgaagatggagtagaagacaacaaattgaaagagaatatggaaagagcttgtttgatgtcgttagatattacagaacatgaactccaaattttggaacagcagtctcaggaagaatatcttagtgatattgcttataaatctactgagcatttatctcccaatgataatgaaaacgatacgtcctatgtaattgagagtgatgaagatttagaaatggagatgcttaagcatttatctcccaatgataatgaaaacgatacgtcctatgtaattgagagtgatgaagatttagaaatggagatgcttaagtctttagaaaacctcaatagtggcacggtagaaccaactcattctaaatgcttaaaaatggaaagaaatctgggtcttcctactaaagaagaagaagaagatgatgaaaatgaagctaatgaaggggaagaagatgatgataaggactttttgtggccagcacccaatgaagagcaagttacttgcctcaagatgtactttggccattccagttttaaaccagttcagtggaaagtgattcattcagtattagaagaaagaagagataatgttgctgtcatggcaactggatatggaaagagtttgtgcttccagtatccacctgtttatgtaggcaagattggccttgttatctctccccttatttctctgatggaagaccaagtgctacagcttaaaatgtccaacatcccagcttgcttccttggatcagcacagtcagaaaatgttctaacagatattaaattaggtaaataccggattgtatacgtaactccagaatactgttcaggtaacatgggcctgctccagcaacttgaggctgatattggtatcacgctcattgctgtggatgaggctcactgtatttctgagtgggggcatgattttagggattcattcaggaagttgggctccctaaagacagcactgccaatggttccaatcgttgcacttactgctactgcaagttcttcaatccgggaagacattgtacgttgcttaaatctgagaaatcctcagatcacctgtactggttttgatcgaccaaacctgtatttagaagttaggcgaaaaacagggaatatccttcaggatctgcagccatttcttgtcaaaacaagttcccactgggaatttgaaggtccaacaatcatctactgtccttctagaaaaatgacacaacaagttacaggtgaacttaggaaactgaatctatcctgtggaacataccatgcgggcatgagttttagcacaaggaaagacattcatcataggtttgtaagagatgaaattcagtgtgtcatagctaccatagcttttggaatgggcattaataaagctgacattcgccaagtcattcattacggtgctcctaaggacatggaatcatattatcaggagattggtagagctggtcgtgatggacttcaaagttcttgtcacgtcctctgggctcctgcagacattaacttaaataggcaccttcttactgagatacgtaatgagaagtttcgattatacaaattaaagatgatggcaaagatggaaaaatatcttcattctagcagatgtaggagacaaatcatcttgtctcattttgaggacaaacaagtacaaaaagcctccttgggaattatgggaactgaaaaatgctgtgataattgcaggtccagattggatcattgctattccatggatgactcagaggatacatcctgggactttggtccacaagcatttaagcttttgtctgctgtggacatcttaggcgaaaaatttggaattgggcttccaattttatttctccgaggatctaattctcagcgtcttgccgatcaatatcgcaggcacagtttatttggcactggcaaggatcaaacagagagttggtggaaggctttttcccgtcagctgatcactgagggattcttggtagaagtttctcggtataacaaatttatgaagatttgcgcccttacgaaaaagggtagaaattggcttcataaagctaatacagaatctcagagcctcatccttcaagctaatgaagaattgtgtccaaagaagttgcttctgcctagttcgaaaactgtatcttcgggcaccaaagagcattgttataatcaagtaccagttgaattaagtacagagaagaagtctaacttggagaagttatattcttataaaccatgtgataagatttcttctgggagtaacatttctaaaaaaagtatcatggtacagtcaccagaaaaagcttacagttcctcacagcctgttatttcggcacaagagcaggagactcagattgtgttatatggcaaattggtagaagctaggcagaaacatgccaataaaatggatgttcccccagctattctggcaacaaacaagatactggtggatatggccaaaatgagaccaactacggttgaaaacgtaaaaaggattgatggtgtttctgaaggcaaagctgccatgttggcccctctgttggaagtcatcaaacatttctgccaaacaaatagtgttcagacagacctcttttcaagtacaaaacctcaagaagaacagaagacgagtctggtagcaaaaaataaaatatgcacactttcacagtctatggccatcacatactctttattccaagaaaagaagatgcctttgaagagcatagctgagagcaggattctgcctctcatgacaattggcatgcacttatcccaagcggtgaaagctggctgcccccttgatttggagcgagcaggcctgactccagaggttcagaagattattgctgatgttatccgaaaccctcccgtcaactcagatatgagtaaaattagcctaatcagaatgttagttcctgaaaacattgacacgtaccttatccacatggcaattgagatccttaaacatggtcctgacagcggacttcaaccttcatgtgatgtcaacaaaaggagatgttttcccggttctgaagagatctgttcaagttctaagagaagcaaggaagaagtaggcatcaatactgagacttcatctgcagagagaaagagacgattacctgtgtggtttgccaaaggaagtgataccagcaagaaattaatggacaaaacgaaaaggggaggtctttttagttaagctggcaattaccagaacaattatgtttcttgctgtattataagaggatagctatattttatttctgaagagtaaggagtagtattttggcttaaaaatcattctaattacaaagttcactgtttattgaagaactggcatcttaaatcagccttccgcaattcatgtagtttctgggtcttctgggagcctacgtgagtacatcacctaacagaatattaaattagacttcctgtaagattgctttaagaaactgttactgtcctgttttctaatctctttattaaaacagtgtatttggaaaatgttatgtgctctgatttgatatagataacagattagtagttacatggtaattatgtgatataaaatattcatatattatcaaaattctgttttgtaaatgtaagaaagcatagttattttacaaattgtttttactgtcttttgaagaagttcttaaatacgttgttaaatggtattagttgaccagggcagtgaaaatgaaaccgcattttgggtgccattaaatagggaaaaaacatgtaaaaaatgtaaaatggagaccaattgcactaggcaagtgtatattttgtattttatatacaatttctattatttttcaagtaataaaacaatgtttttcatactgaatattaaaaaaaaaaaaaaaaaaaa

SEQ ID NO: 9 - protein, 1432 aa, Homo sapiens
MSEKKLETTAQQRKCPEWMNVQNKRCAVEERKACVRKSVFEDDLPFLEFTGSIVYSYDASDCSFLSEDISMSLSDGDVVGFDMEWPPLYNRGKLGKVALIQLCVSESKCYLFHVSSMSVFPQGLKMLLENKAVKKAGVGIEGDQWKLLRDFDIKLKNFVELTDVANKKLKCTETWSLNSLVKHLLGKQLLKDKSIRCSNWSKFPLTEDQKLYAATDAYAGFIIYRNLEILDDTVQRFAINKEEEILLSDMNKQLTSISEEVMDLAKHLPHAFSKLENPRRVSILLKDISENLYSLRRMIIGSTNIETELRPSNNLNLLSFEDSTTGGVQQKQIREHEVLIHVEDETWDPTLDHLAKHDGEDVLGNKVERKEDGFEDGVEDNKLKENMERACLMSLDITEHELQILEQQSQEEYLSDIAYKSTEHLSPNDNENDTSYVIESDEDLEMEMLKHLSPNDNENDTSYVIESDEDLEMEMLKSLENLNSGTVEPTHSKCLKMERNLGLPTKEEEEDDENEANEGEEDDDKDFLWPAPNEEQVTCLKMYFGHSSFKPVQWKVIHSVLEERRDNVAVMATGYGKSLCFQYPPVYVGKIGLVISPLISLMEDQVLQLKMSNIPACFLGSAQSENVLTDIKLGKYRIVYVTPEYCSGNMGLLQQLEADIGITLIAVDEAHCISEWGHDFRDSFRKLGSLKTALPMVPIVALTAIASSSIREDIVRCLNLRNPQITCTGFDRPNLYLEVRRKTGNILQDLQPFLVKTSSHWEFEGPTIIYCPSRKMTQQVTGELRKLNLSCGTYHAGMSFSTRKDIHHRFVRDEIQCVIATIAFGMGINKADIRQVIHYGAPKDMESYYQEIGRAGRDGLQSSCHVLWAPADINLNRHLLTEIRNEKFRLYKLKMMAKMEKYLHSSRCRRQIILSHFEDKQVQKASLGIMGTEKCCDNCRSRLDHCYSMDDSEDTSWDFGPQAFKLLSAVDILGEKFGIGLPILFLRGSNSQRLADQYRRHSLFGTGKDQTESWWKAFSRQLITEGFLVEVSRYNKFMKICALTKKGRNWLHKANTESQSLILQANEELCPKKLLLPSSKTVSSGTKEHCYNQVPVELSTEKKSNLEKLYSYKPCDKISSGSNISKKSIMVQSPEKAYSSSQPVISAQEQETQIVLYGKLVEARQKHANKMDVPPAILATNKILVDMAKMRPTTVENVKRIDGVSEGKAAMLAPLLEVIKHFCQTNSVQTDLFSSTKPQEEQKTSLVAKNKICTLSQSMAITYSLFQEKKMPLKSIAESRILPLMTIGMHLSQAVKAGCPLDLERAGLTPEVQKIIADVIRNPPVNSDMSKISLIRMLVPENIDTYLIHMAIEILKHGPDSGLQPSCDVNKRRCFPGSEEICSSSKRSKEEVGINTETSSAERKRRLPVWFAKGSDTSKKLMDKTKRGGLFS

SEQ ID NO: 10 - protein, 1432 aa, Homo sapiens
MSEKKLETTAQQRKCPEWMNVQNKRCAVEERKACVRKSVFEDDLPFLEFTGSIVYSYDASDCSFLSEDISMSLSDGDVVGFDMEWPPLYNRGKLGKVALIQLCVSESKCYLFHVSSMSVFPQGLKMLLENKAVKKAGVGIEGDQWKLLRDFDIKLKNFVELTDVANKKLKCTETWSLNSLVKHLLGKQLLKDKSIRCSNWSKFPLTEDQKLYAATDAYAGFIIYRNLEILDDTVQRFAINKEEEILLSDMNKQLTSISEEVMDLAKHLPHAFSKLENPRRVSILLKDISENLYSLRRMIIGSTNIETELRPSNNLNLLSFEDSTTGGVQQKQIREHEVLIHVEDETWDPTLDHLAKHDGEDVLGNKVERKEDGFEDGVEDNKLKENMERACLMSLDITEHELQILEQQSQEEYLSDIAYKSTEHLSPNDNENDTSYVIESDEDLEMEMLKHLSPNDNENDTSYVIESDEDLEMEMLKSLENLNSGTVEPTHSKCLKMERNLGLPTKEEEEDDENEANEGEEDDDKDFLWPAPNEEQVTCLKMYFGHSSFKPVQWKVIHSVLEERRDNVAVMATGYGKSLCFQYPPVYVGKIGLVISPLISLMEDQVLQLKMSNIPACFLGSAQSENVLTDIKLGKYRIVYVTPEYCSGNMGLLQQLEADIGITLIAVDEAHCISEWGHDFRDSFRKLGSLKTALPMVPIVALTATASSSIREDIVRCLNLRNPQITCTGFDRPNLYLEVRRKTGNILQDLQPFLVKTSSHWEFEGPTIIYCPSRKMTQQVTGELRKLNLSCGTYHAGMSFSTRKDIHHRFVRDEIQCVIATIAFGMGINKADIRQVIHYGAPKDMESYYQEIGRAGRDGLQSSCHVLWAPADINLNRHLLTEIRNEKFRLYKLKMMAKMEKYLHSSRCRRQIILSHFEDKQVQKASLGIMGTEKCCDNCRSRLDHCYSMDDSEDTSWDFGPQAFKLLSAVDILGEKFGIGLPILFLRGSNSQRLADQYRRHSLFGTGKDQTESWWKAFSRQLITEGFLVEVSRYNKFMKICALTKKGRNWLHKANTESQSLILQANEELCPKKLLLPSSKTVSSGTKEHCYNQVPVELSTEKKSNLEKLYSYKPCDKISSGSNISKKSIMVQSPEKAYSSSQPVISAQEQETQIVLYGKLVEARQKHANKMDVPPAILATNKILVDMAKMRPTTVENVKRIDGVSEGKAAMLAPLLEVIKHFCQTNSVQTDLFSSTKPQEEQKTSLVAKNKICTLSQSMAITYSLFQEKKMPLKSIAESRILPLMTIGMHLSQAVKAGCPLDLERAGLTPEVQKIIADVIRNPPVNSDMSKISLIRMLVPENIDTYLIHMAIEILKHGPDSGLQPSCDVNKRRCFPGSEEICSSSKRSKEEVGINTETSSAERKRRLPVWFAKGSDTSKKLMDKTKRGGLFS

SEQ ID NO: 11 - DNA, 4296 bp, Homo sapiens
atgagtgaaaaaaaattggaaacaactgcacagcagcggaaatgtcctgaatggatgaatgtgcagaataaaagatgtgctgtagaagaaagaaaggcatgtgttcggaagagtgtttttgaagatgacctccccttcttagaattcactggatccattgtgtatagttacgatgctagtgattgctctttcctgtcagaagatattagcatgagtctatcagatggggatgtggtgggatttgacatggagtggccaccattatacaatagagggaaacttggcaaagttgcactaattcagttgtgtgtttctgagagcaaatgttacttgttccacgtttcttccatgtcagtttttccccagggattaaaaatgttgcttgaaaataaagcagttaaaaaggcaggtgtaggaattgaaggagatcagtggaaacttctacgtgactttgatatcaaattgaagaattttgtggagttgacagatgttgccaataaaaagctgaaatgcacagagacctggagccttaacagtctggttaaacacctcttaggtaaacagctcctgaaagacaagtctatccgctgtagcaattggagtaaatttcctctcactgaggaccagaaactgtatgcagccactgatgcttatgctggttttattatttaccgaaatttagagattttggatgatactgtgcaaaggtttgctataaataaagaggaagaaatcctacttagcgacatgaacaaacagttgacttcaatctctgaggaagtgatggatctggctaagcatcttcctcatgctttcagtaaattggaaaacccacggagggtttctatcttactaaaggatatttcagaaaatctatattcactgaggaggatgataattgggtctactaacattgagactgaactgaggcccagcaataatttaaacttattatcctttgaagattcaactactgggggagtacaacagaaacaaattagagaacatgaagttttaattcacgttgaagatgaaacatgggacccaacacttgatcatttagctaaacatgatggagaagatgtacttggaaataaagtggaacgaaaagaagatggatttgaagatggagtagaagacaacaaattgaaagagaatatggaaagagcttgtttgatgtcgttagatattacagaacatgaactccaaattttggaacagcagtctcaggaagaatatcttagtgatattgcttataaatctactgagcatttatctcccaatgataatgaaaacgatacgtcctatgtaattgagagtgatgaagatttagaaatggagatgcttaagcatttatctcccaatgataatgaaaacgatacgtcctatgtaattgagagtgatgaagatttagaaatggagatgcttaagtctttagaaaacctcaatagtggcacggtagaaccaactcattctaaatgcttaaaaatggaaagaaatctgggtcttcctactaaagaagaagaagaagatgatgaaaatgaagctaatgaaggggaagaagatgatgataaggactttttgtggccagcacccaatgaagagcaagttacttgcctcaagatgtactttggccattccagttttaaaccagttcagtggaaagtgattcattcagtattagaagaaagaagagataatgttgctgtcatggcaactggatatggaaagagtttgtgcttccagtatccacctgtttatgtaggcaagattggccttgttatctctccccttatttctctgatggaagaccaagtgctacagcttaaaatgtccaacatcccagcttgcttccttggatcagcacagtcagaaaatgttctaacagatattaaattaggtaaataccggattgtatacgtaactccagaatactgttcaggtaacatgggcctgctccagcaacttgaggctgatattggtatcacgctcattgctgtggatgaggctcactgtatttctgagtgggggcatgattttagggattcattcaggaagttgggctccctaaagacagcactgccaatggttccaatcgttgcacttactgctactgcaagttcttcaatccgggaagacattgtacgttgcttaaatctgagaaatcctcagatcacctgtactggttttgatcgaccaaacctgtatttagaagttaggcgaaaaacagggaatatccttcaggatctgcagccatttcttgtcaaaacaagttcccactgggaatttgaaggtccaacaatcatctactgtccttctagaaaaatgacacaacaagttacaggtgaacttaggaaactgaatctatcctgtggaacataccatgcgggcatgagttttagcacaaggaaagacattcatcataggtttgtaagagatgaaattcagtgtgtcatagctaccatagcttttggaatgggcattaataaagctgacattcgccaagtcattcattacggtgctcctaaggacatggaatcatattatcaggagattggtagagctggtcgtgatggacttcaaagttcttgtcacgtcctctgggctcctgcagacattaacttaaataggcaccttcttactgagatacgtaatgagaagtttcgattatacaaattaaagatgatggcaaagatggaaaaatatcttcattctagcagatgtaggagacaaatcatcttgtctcattttgaggacaaacaagtacaaaaagcctccttgggaattatgggaactgaaaaatgctgtgataattgcaggtccagattggatcattgctattccatggatgactcagaggatacatcctgggactttggtccacaagcatttaagcttttgtctgctgtggacatcttaggcgaaaaatttggaattgggcttccaattttatttctccgaggatctaattctcagcgtcttgccgatcaatatcgcaggcacagtttatttggcactggcaaggatcaaacagagagttggtggaaggctttttcccgtcagctgatcactgagggattcttggtagaagtttctcggtataacaaatttatgaagatttgcgcccttacgaaaaagggtagaaattggcttcataaagctaatacagaatctcagagcctcatccttcaagctaatgaagaattgtgtccaaagaagttgcttctgcctagttcgaaaactgtatcttcgggcaccaaagagcattgttataatcaagtaccagttgaattaagtacagagaagaagtctaacttggagaagttatattcttataaaccatgtgataagatttcttctgggagtaacatttctaaaaaaagtatcatggtacagtcaccagaaaaagcttacagttcctcacagcctgttatttcggcacaagagcaggagactcagattgtgttatatggcaaattggtagaagctaggcagaaacatgccaataaaatggatgttcccccagctattctggcaacaaacaagatactggtggatatggccaaaatgagaccaactacggttgaaaacgtaaaaaggattgatggtgtttctgaaggcaaagctgccatgttggcccctctgttggaagtcatcaaacatttctgccaaacaaatagtgttcagacagacctcttttcaagtacaaaacctcaagaagaacagaagacgagtctggtagcaaaaaataaaatatgcacactttcacagtctatggccatcacatactctttattccaagaaaagaagatgcctttgaagagcatagctgagagcaggattctgcctctcatgacaattggcatgcacttataccaagcggtgaaagctggctgcccccttgatttggagcgagcaggcctgactccagaggttcagaagattattgctgatgttatccgaaaccctcccgtcaactcagatatgagtaaaattagcctaatcagaatgttagttcctgaaaacattgacacgtaccttatccacatggcaattgagatccttaaacatggtcctgacagcggacttcaaccttcatgtgatgtcaacaaaaggagatgttttcccggttctgaagagatctgttcaagttctaagagaagcaaggaagaagtaggcatcaatactgagacttcatctgcagagagaaagagacgattacctgtgtggtttgccaaaggaagtgataccagcaagaaattaatggacaaaacgaaaaggggaggtctttttagt

SEQ ID NO: 12 - protein, 1432 aa, Homo sapiens
MSEKKLETTAQQRKCPEWMNVQNKRCAVEERKACVRKSVFEDDLPFLEFTGSIVYSYDASDCSFLSEDISMSLSDGDVVGFDMEWPPLYNRGKLGKVALIQLCVSESKCYLFHVSSMSVFPQGLKMLLENKAVKKAGVGIEGDQWKLLRDFDIKLKNFVELTDVANKKLKCTETWSLNSLVKHLLGKQLLKDKSIRCSNWSKFPLTEDQKLYAATDAYAGFIIYRNLEILDDTVQRFAINKEEEILLSDMNKQLTSISEEVMDLAKHLPHAFSKLENPRRVSILLKDISENLYSLRRMIIGSTNIETELRPSNNLNLLSFEDSTTGGVQQKQIREHEVLIHVEDETWDPTLDHLAKHDGEDVLGNKVERKEDGFEDGVEDNKLKENMERACLMSLDITEHELQILEQQSQEEYLSDIAYKSTEHLSPNDNENDTSYVIESDEDLEMEMLKHLSPNDNENDTSYVIESDEDLEMEMLKSLENLNSGTVEPTHSKCLKMERNLGLPTKEEEEDDENEANEGEEDDDKDFLWPAPNEEQVTCLKMYFGHSSFKPVQWKVIHSVLEERRDNVAVMATGYGKSLCFQYPPVYVGKIGLVISPLISLMEDQVLQLKMSNIPACFLGSAQSENVLTDIKLGKYRIVYVTPEYCSGNMGLLQQLEADIGITLIAVDEAHCISEWGHDFRDSFRKLGSLKTALPMVPIVALTATASSSIREDIVRCLNLRNPQITCTGFDRPNLYLEVRRKTGNILQDLQPFLVKTSSHWEFEGPTIIYCPSRKMTQQVTGELRKLNLSCGTYHAGMSFSTRKDIHHRFVRDEIQCVIATIAFGMGINKADIRQVIHYGAPKDMESYYQEIGRAGRDGLQSSCHVLWAPADINLNRHLLTEIRNEKFRLYKLKMMAKMEKYLHSSRCRRQIILSHFEDKQVQKASLGIMGTEKCCDNCRSRLDHCYSMDDSEDTSWDFGPQAFKLLSAVDILGEKFGIGLPILFLRGSNSQRLADQYRRHSLFGTGKDQTESWWKAFSRQLITEGFLVEVSRYNKFMKICALTKKGRNWLHKANTESQSLILQANEELCPKKLLLPSSKTVSSGTKEHCYNQVPVELSTEKKSNLEKLYSYKPCDKISSGSNISKKSIMVQSPEKAYSSSQPVISAQEQETQIVLYGKLVEARQKHANKMDVPPAILATNKILVDMAKMRPTTVENVKRIDGVSEGKAAMLAPLLEVIKHFCQTNSVQTDLFSSTKPQEEQKTSLVAKNKICTLSQSMAITYSLFQEKKMPLKSIAESRILPLMTIGMHLYQAVKAGCPLDLERAGLTPEVQKIIADVIRNPPVNSDMSKISLIRMLVPENIDTYLIHMAIEILKHGPDSGLQPSCDVNKRRCFPGSEEICSSSKRSKEEVGINTETSSAERKRRLPVWFAKGSDTSKKLMDKTKRGGLFS

SEQ ID NO: 13 - DNA, 4296 bp, Homo sapiens
atgagtgaaaaaaaattggaaacaactgcacagcagcggaaatgtcctgaatggatgaatgtgcagaataaaagatgtgctgtagaagaaagaaaggcatgtgttcggaagagtgtttttgaagatgacctccccttcttagaattcactggatccattgtgtatagttacgatgctagtgattgctctttcctgtcagaagatattagcatgagtctatcagatggggatgtggtgggatttgacatggagtggccaccattatacaatagagggaaacttggcaaagttgcactaattcagttgtgtgtttctgagagcaaatgttacttgttccacgtttcttccatgtcagtttttccccagggattaaaaatgttgcttgaaaataaagcagttaaaaaggcaggtgtaggaattgaaggagatcagtggaaacttctacgtgactttgatatcaaattgaagaattttgtggagttgacagatgttgccaataaaaagctgaaatgcacagagacctggagccttaacagtctggttaaacacctcttaggtaaacagctcctgaaagacaagtctatccgctgtagcaattggagtaaatttcctctcactgaggaccagaaactgtatgcagccactgatgcttatgctggttttattatttaccgaaatttagagattttggatgatactgtgcaaaggtttgctataaataaagaggaagaaatcctacttagcgacatgaacaaacagttgacttcaatctctgaggaagtgatggatctggctaagcatcttcctcatgctttcagtaaattggaaaacccacggagggtttctatcttactaaaggatatttcagaaaatctatattcactgaggaggatgataattgggtctactaacattgagactgaactgaggcccagcaataatttaaacttattatcctttgaagattcaactactgggggagtacaacagaaacaaattagagaacatgaagttttaattcacgttgaagatgaaacatgggacccaacacttgatcatttagctaaacatgatggagaagatgtacttggaaataaagtggaacgaaaagaagatggatttgaagatggagtagaagacaacaaattgaaagagaatatggaaagagcttgtttgatgtcgttagatattacagaacatgaactccaaattttggaacagcagtctcaggaagaatatcttagtgatattgcttataaatctactgagcatttatctcccaatgataatgaaaacgatacgtcctatgtaattgagagtgatgaagatttagaaatggagatgcttaagcatttatctcccaatgataatgaaaacgatacgtcctatgtaattgagagtgatgaagatttagaaatggagatgcttaagtctttagaaaacctcaatagtggcacggtagaaccaactcattctaaatgcttaaaaatggaaagaaatctgggtcttcctactaaagaagaagaagaagatgatgaaaatgaagctaatgaaggggaagaagatgatgataaggactttttgtggccagcacccaatgaagagcaagttacttgcctcaagatgtactttggccattccagttttaaaccagttcagtggaaagtgattcattcagtattagaagaaagaagagataatgttgctgtcatggcaactggatatggaaagagtttgtgcttccagtatccacctgtttatgtaggcaagattggccttgttatctctccccttatttctctgatggaagaccaagtgctacagcttaaaatgtccaacatcccagcttgcttccttggatcagcacagtcagaaaatgttctaacagatattaaattaggtaaataccggattgtatacgtaactccagaatactgttcaggtaacatgggcctgctccagcaacttgaggctgatattggtatcacgctcattgctgtggatgaggctcactgtatttctgagtgggggcatgattttagggattcattcaggaagttgggctccctaaagacagcactgccaatggttccaatcgttgcacttactgctattgcaagttcttcaatccgggaagacattgtacgttgcttaaatctgagaaatcctcagatcacctgtactggttttgatcgaccaaacctgtatttagaagttaggcgaaaaacagggaatatccttcaggatctgcagccatttcttgtcaaaacaagttcccactgggaatttgaaggtccaacaatcatctactgtccttctagaaaaatgacacaacaagttacaggtgaacttaggaaactgaatctatcctgtggaacataccatgcgggcatgagttttagcacaaggaaagacattcatcataggtttgtaagagatgaaattcagtgtgtcatagctaccatagcttttggaatgggcattaataaagctgacattcgccaagtcattcattacggtgctcctaaggacatggaatcatattatcaggagattggtagagctggtcgtgatggacttcaaagttcttgtcacgtcctctgggctcctgcagacattaacttaaataggcaccttcttactgagatacgtaatgagaagtttcgattatacaaattaaagatgatggcaaagatggaaaaatatcttcattctagcagatgtaggagacaaatcatcttgtctcattttgaggacaaacaagtacaaaaagcctccttgggaattatgggaactgaaaaatgctgtgataattgcaggtccagattggatcattgctattccatggatgactcagaggatacatcctgggactttggtccacaagcatttaagcttttgtctgctgtggacatcttaggcgaaaaatttggaattgggcttccaattttatttctccgaggatctaattctcagcgtcttgccgatcaatatcgcaggcacagtttatttggcactggcaaggatcaaacagagagttggtggaaggctttttcccgtcagctgatcactgagggattcttggtagaagtttctcggtataacaaatttatgaagatttgcgcccttacgaaaaagggtagaaattggcttcataaagctaatacagaatctcagagcctcatccttcaagctaatgaagaattgtgtccaaagaagttgcttctgcctagttcgaaaactgtatcttcgggcaccaaagagcattgttataatcaagtaccagttgaattaagtacagagaagaagtctaacttggagaagttatattcttataaaccatgtgataagatttcttctgggagtaacatttctaaaaaaagtatcatggtacagtcaccagaaaaagcttacagttcctcacagcctgttatttcggcacaagagcaggagactcagattgtgttatatggcaaattggtagaagctaggcagaaacatgccaataaaatggatgttcccccagctattctggcaacaaacaagatactggtggatatggccaaaatgagaccaactacggttgaaaacgtaaaaaggattgatggtgtttctgaaggcaaagctgccatgttggcccctctgttggaagtcatcaaacatttctgccaaacaaatagtgttcagacagacctcttttcaagtacaaaacctcaagaagaacagaagacgagtctggtagcaaaaaataaaatatgcacactttcacagtctatggccatcacatactctttattccaagaaaagaagatgcctttgaagagcatagctgagagcaggattctgcctctcatgacaattggcatgcacttataccaagcggtgaaagctggctgcccccttgatttggagcgagcaggcctgactccagaggttcagaagattattgctgatgttatccgaaaccctcccgtcaactcagatatgagtaaaattagcctaatcagaatgttagttcctgaaaacattgacacgtaccttatccacatggcaattgagatccttaaacatggtcctgacagcggacttcaaccttcatgtgatgtcaacaaaaggagatgttttcccggttctgaagagatctgttcaagttctaagagaagcaaggaagaagtaggcatcaatactgagacttcatctgcagagagaaagagacgattacctgtgtggtttgccaaaggaagtgataccagcaagaaattaatggacaaaacgaaaaggggaggtctttttagt

SEQ ID NO: 14 - protein, 1432 aa, Homo sapiens
MSEKKLETTAQQRKCPEWMNVQNKRCAVEERKACVRKSVFEDDLPFLEFTGSIVYSYDASDCSFLSEDISMSLSDGDVVGFDMEWPPLYNRGKLGKVALIQLCVSESKCYLFHVSSMSVFPQGLKMLLENKAVKKAGVGIEGDQWKLLRDFDIKLKNFVELTDVANKKLKCTETWSLNSLVKHLLGKQLLKDKSIRCSNWSKFPLTEDQKLYAATDAYAGFIIYRNLEILDDTVQRFAINKEEEILLSDMNKQLTSISEEVMDLAKHLPHAFSKLENPRRVSILLKDISENLYSLRRMIIGSTNIETELRPSNNLNLLSFEDSTTGGVQQKQIREHEVLIHVEDETWDPTLDHLAKHDGEDVLGNKVERKEDGFEDGVEDNKLKENMERACLMSLDITEHELQILEQQSQEEYLSDIAYKSTEHLSPNDNENDTSYVIESDEDLEMEMLKHLSPNDNENDTSYVIESDEDLEMEMLKSLENLNSGTVEPTHSKCLKMERNLGLPTKEEEEDDENEANEGEEDDDKDFLWPAPNEEQVTCLKMYFGHSSFKPVQWKVIHSVLEERRDNVAVMATGYGKSLCFQYPPVYVGKIGLVISPLISLMEDQVLQLKMSNIPACFLGSAQSENVLTDIKLGKYRIVYVTPEYCSGNMGLLQQLEADIGITLIAVDEAHCISEWGHDFRDSFRKLGSLKTALPMVPIVALTAIASSSIREDIVRCLNLRNPQITCTGFDRPNLYLEVRRKTGNILQDLQPFLVKTSSHWEFEGPTIIYCPSRKMTQQVTGELRKLNLSCGTYHAGMSFSTRKDIHHRFVRDEIQCVIATIAFGMGINKADIRQVIHYGAPKDMESYYQEIGRAGRDGLQSSCHVLWAPADINLNRHLLTEIRNEKFRLYKLKMMAKMEKYLHSSRCRRQIILSHFEDKQVQKASLGIMGTEKCCDNCRSRLDHCYSMDDSEDTSWDFGPQAFKLLSAVDILGEKFGIGLPILFLRGSNSQRLADQYRRHSLFGTGKDQTESWWKAFSRQLITEGFLVEVSRYNKFMKICALTKKGRNWLHKANTESQSLILQANEELCPKKLLLPSSKTVSSGTKEHCYNQVPVELSTEKKSNLEKLYSYKPCDKISSGSNISKKSIMVQSPEKAYSSSQPVISAQEQETQIVLYGKLVEARQKHANKMDVPPAILATNKILVDMAKMRPTTVENVKRIDGVSEGKAAMLAPLLEVIKHFCQTNSVQTDLFSSTKPQEEQKTSLVAKNKICTLSQSMAITYSLFQEKKMPLKSIAESRILPLMTIGMHLYQAVKAGCPLDLERAGLTPEVQKIIADVIRNPPVNSDMSKISLIRMLVPENIDTYLIHMAIEILKHGPDSGLQPSCDVNKRRCFPGSEEICSSSKRSKEEVGINTETSSAERKRRLPVWFAKGSDTSKKLMDKTKRGGLFS

SEQ ID NO: 15 - DNA, 3396 bp, Homo sapiens
atgccgcgcgctccccgctgccgagccgtgcgctccctgctgcgcagccactaccgcgaggtgctgccgctggccacgttcgtgcggcgcctggggccccagggctggcggctggtgcagcgcggggacccggcggctttccgcgcgctggtggcccagtgcctggtgtgcgtgccctgggacgcacggccgccccccgccgccccctccttccgccaggtgtcctgcctgaaggagctggtggcccgagtgctgcagaggctgtgcgagcgcggcgcgaagaacgtgctggccttcggcttcgcgctgctggacggggcccgcgggggcccccccgaggccttcaccaccagcgtgcgcagctacctgcccaacacggtgaccgacgcactgcgggggagcggggcgtgggggctgctgctgcgccgcgtgggcgacgacgtgctggttcacctgctggcacgctgcgcgctctttgtgctggtggctcccagctgcgcctaccaggtgtgcgggccgccgctgtaccagctcggcgctgccactcaggcccggcccccgccacacgctagtggaccccgaaggcgtctgcgatgcgaacgggcctggaaccatagcgtcagggaggccggggtccccctgggcctgccagccccgggtgcgaggaggcgcgggggcagtgccagccgaagtctgccgttgcccaagaggcccaggcgtggcgctgcccctgagccggagcggacgcccgttgggcaggggtcctgggcccacccgggcaggacgcgtggaccgagtgaccgtggtttctgtgtggtgtcacctgccagacccgccgaagaagccacctctttggagggtgcgctctctggcacgcgccactcccacccatccgtgggccgccagcaccacgcgggccccccatccacatcgcggccaccacgtccctgggacacgccttgtcccccggtgtacgccgagaccaagcacttcctctactcctcaggcgacaaggagcagctgcggccctccttcctactcagctctctgaggcccagcctgactggcgctcggaggctcgtggagaccatctttctgggttccaggccctggatgccagggactccccgcaggttgccccgcctgccccagcgctactggcaaatgcggcccctgtttctggagctgcttgggaaccacgcgcagtgcccctacggggtgctcctcaagacgcactgcccgctgcgagctgcggtcaccccagcagccggtgtctgtgcccgggagaagccccagggctctgtggcggcccccgaggaggaggacacagacccccgtcgcctggtgcagctgctccgccagcacagcagcccctggcaggtgtacggcttcgtgcgggcctgcctgcgccggctggtgcccccaggcctctggggctccaggcacaacgaacgccgcttcctcaggaacaccaagaagttcatctccctggggaagcatgccaagctctcgctgcaggagctgacgtggaagatgagcgtgcgggactgcgcttggctgcgcaggagcccaggggttggctgtgttccggccgcagagcaccgtctgcgtgaggagatcctggccaagttcctgcactggctgatgagtgtgtacgtcgtcgagctgctcaggtctttcttttatgtcacggagaccacgtttcaaaagaacaggctctttttctaccggaagagtgtctggagcaagttgcaaagcattggaatcagacagcacttgaagagggtgcagctgcgggagctgtcggaagcagaggtcaggcagcatcgggaagccaggcccgccctgctgacgtccagactccgcttcatccccaagcctgacgggctgcggccgattgtgaacatggactacgtcgtgggagccagaacgttccgcagagaaaagagggccgagcgtctcacctcgagggtgaaggcactgttcagcgtgctcaactacgagcgggcgcggcgccccggcctcctgggcgcctctgtgctgggcctggacgatatccacagggcctggcgcaccttcgtgctgcgtgtgcgggcccaggacccgccgcctgagctgtactttgtcaaggtggatgtgacgggcgcgtacgacaccatcccccaggacaggctcacggaggtcatcgccagcatcatcaaaccccagaacacgtactgcgtgcgtcggtatgccgtggtccagaaggccgcccatgggcacgtccgcaaggccttcaagagccacgtctctaccttgacagacctccagccgtacatgcgacagttcgtggctcacctgcaggagaccagcccgctgagggatgccgtcgtcatcgagcagagctcctccctgaatgaggccagcagtggcctcttcgacgtcttcctacgcttcatgtgccaccacgccgtgcgcatcaggggcaagtcctacgtccagtgccaggggatcccgcagggctccatcctctccacgctgctctgcagcctgtgctacggcgacatggagaacaagctgtttgcggggattcggcgggacgggctgctcctgcgtttggtggatgatttcttgttggtgacacctcacctcacccacgcgaaaaccttcctcaggaccctggtccgaggtgtccctgagtatggctgcgtggtgaacttgcggaagacagtggtgaacttccctgtagaagacgaggccctgggtggcacggcttttgttcagatgccggcccacggcctattcccctggtgcggcctgctgctggatacccggaccctggaggtgcagagcgactactccagctatgcccggacctccatcagagccagtctcaccttcaaccgcggcttcaaggctgggaggaacatgcgtcgcaaactctttggggtcttgcggctgaagtgtcacagcctgtttctggatttgcaggtgaacagcctccagacggtgtgcaccaacatctacaagatcctcctgctgcaggcgtacaggtttcacgcatgtgtgctgcagctcccatttcatcagcaagtttggaagaaccccacatttttcctgcgcgtcatctctgacacggcctccctctgctactccatcctgaaagccaagaacgcagggatgtcgctgggggccaagggcgccgccggccctctgccctccgaggccgtgcagtggctgtgccaccaagcattcctgctcaagctgactcgacaccgtgtcacctacgtgccactcctggggtcactcaggacagcccagacgcagctgagtcggaagctcccggggacgacgctgactgccctggaggccgcagccaacccggcactgccctcagacttcaagaccatcctggac

SEQ ID NO: 16 - DNA, 3396 bp, Homo sapiens
atgccgcgcgctccccgctgccgagccgtgcgctccctgctgcgcagccactaccgcgaggtgctgccgctggccacgttcgtgcggcgcctggggccccagggctggcggctggtgcagcgcggggacccggcggctttccgcgcgctggtggcccagtgcctggtgtgcgtgccctgggacgcacggccgccccccgccgccccctccttccgccaggtgtcctgcctgaaggagctggtggcccgagtgctgcagaggctgtgcgagcgcggcgcgaagaacgtgctggccttcggcttcgcgctgctggacggggcccgcgggggcccccccgaggccttcaccaccagcgtgcgcagctacctgcccaacacggtgaccgacgcactgcgggggagcggggcgtgggggctgctgctgcgccgcgtgggcgacgacgtgctggttcacctgctggcacgctgcgcgctctttgtgctggtggctcccagctgcgcctaccaggtgtgcgggccgccgctgtaccagctcggcgctgccactcaggcccggcccccgccacacgctagtggaccccgaaggcgtctgggatgcgaacgggcctggaaccatagcgtcagggaggccggggtccccctgggcctgccagccccgggtgcgaggaggcgcgggggcagtgccagccgaagtctgccgttgcccaagaggcccaggcgtggcgctgcccctgagccggagcggacgcccgttgggcaggggtcctgggcccacccgggcaggacgcgtggaccgagtgaccgtggtttctgtgtggtgtcacctgccagacccgccgaagaagccacctctttggagggtgcgctctctggcacgcgccactcccacccatccgtgggccgccagcaccacgcgggccccccatccacatcgcggccaccacgtccctgggacacgccttgtcccccggtgtacgccgagaccaagcacttcctctactcctcaggcgacaaggagcagctgcggccctccttcctactcagctctctgaggcccagcctgactggcgctcggaggctcgtggagaccatctttctgggttccaggccctggatgccagggactccccgcaggttgccccgcctgccccagcgctactggcaaatgcggcccctgtttctggagctgcttgggaaccacgcgcagtgcccctacggggtgctcctcaagacgcactgcccgctgcgagctgcggtcaccccagcagccggtgtctgtgcccgggagaagccccagggctctgtggcggcccccgaggaggaggacacagacccccgtcgcctggtgcagctgctccgccagcacagcagcccctggcaggtgtacggcttcgtgcgggcctgcctgcgccggctggtgcccccaggcctctggggctccaggcacaacgaacgccgcttcctcaggaacaccaagaagttcatctccctggggaagcatgccaagctctcgctgcaggagctgacgtggaagatgagcgtgcgggactgcgcttggctgcgcaggagcccaggggttggctgtgttccggccgcagagcaccgtctgcgtgaggagatcctggccaagttcctgcactggctgatgagtgtgtacgtcgtcgagctgctcaggtctttcttttatgtcacggagaccacgtttcaaaagaacaggctctttttctaccggaagagtgtctggagcaagttgcaaagcattggaatcagacagcacttgaagagggtgcagctgcgggagctgtcggaagcagaggtcaggcagcatcgggaagccaggcccgccctgctgacgtccagactccgcttcatccccaagcctgacgggctgcggccgattgtgaacatggactacgtcgtgggagccagaacgttccgcagagaaaagagggccgagcgtctcacctcgagggtgaaggcactgttcagcgtgctcaactacgagcgggcgcggcgccccggcctcctgggcgcctctgtgctgggcctggacgatatccacagggcctggcgcaccttcgtgctgcgtgtgcgggcccaggacccgccgcctgagctgtactttgtcaaggtggatgtgacgggcgcgtacgacaccatcccccaggacaggctcacggaggtcatcgccagcatcatcaaaccccagaacacgtactgcgtgcgtcggtatgccgtggtccagaaggccgcccatgggcacgtccgcaaggccttcaagagccacgtctctaccttgacagacctccagccgtacatgcgacagttcgtggctcacctgcaggagaccagcccgctgagggatgccgtcgtcatcgagcagagctcctccctgaatgaggccagcagtggcctcttcgacgtcttcctacgcttcatgtgccaccacgccgtgcgcatcaggggcaagtcctacgtccagtgccaggggatcccgcagggctccatcctctccacgctgctctgcagcctgtgctacggcgacatggagaacaagctgtttgcggggattcggcgggacgggctgctcctgcgtttggtggatgatttcttgttggtgacacctcacctcacccacgcgaaaaccttcctcaggaccctggtccgaggtgtccctgagtatggctgcgtggtgaacttgcggaagacagtggtgaacttccctgtagaagacgaggccctgggtggcacggcttttgttcagatgccggcccacggcctattcccctggtgcggcctgctgctggatacccggaccctggaggtgcagagcgactactccagctatgcccggacctccatcagagccagtctcaccttcaaccgcggcttcaaggctgggaggaacatgcgtcgcaaactctttggggtcttgcggctgaagtgtcacagcctgtttctggatttgcaggtgaacagcctccagacggtgtgcaccaacatctacaagatcctcctgctgcaggcgtacaggtttcacgcatgtgtgctgcagctcccatttcatcagcaagtttggaagaaccccacatttttcctgcgcgtcatctctgacacggcctccctctgctactccatcctgaaagccaagaacgcagggatgtcgctgggggccaagggcgccgccggccctctgccctccgaggccgtgcagtggctgtgccaccaagcattcctgctcaagctgactcgacaccgtgtcacctacgtgccactcctggggtcactcaggacagcccagacgcagctgagtcggaagctcccggggacgacgctgactgccctggaggccgcagccaacccggcactgccctcagacttcaagaccatcctggac

SEQ ID NO: 17 - DNA, 4018 bp, Homo sapiens
caggcagcgctgcgtcctgctgcgcacgtgggaagccctggccccggccacccccgcgatgccgcgcgctccccgctgccgagccgtgcgctccctgctgcgcagccactaccgcgaggtgctgccgctggccacgttcgtgcggcgcctggggccccagggctggcggctggtgcagcgcggggacccggcggctttccgcgcgctggtggcccagtgcctggtgtgcgtgccctgggacgcacggccgccccccgccgccccctccttccgccaggtgtcctgcctgaaggagctggtggcccgagtgctgcagaggctgtgcgagcgcggcgcgaagaacgtgctggccttcggcttcgcgctgctggacggggcccgcgggggcccccccgaggccttcaccaccagcgtgcgcagctacctgcccaacacggtgaccgacgcactgcgggggagcggggcgtgggggctgctgctgcgccgcgtgggcgacgacgtgctggttcacctgctggcacgctgcgcgctctttgtgctggtggctcccagctgcgcctaccaggtgtgcgggccgccgctgtaccagctcggcgctgccactcaggcccggcccccgccacacgctagtggaccccgaaggcgtctgggatgcgaacgggcctggaaccatagcgtcagggaggccggggtccccctgggcctgccagccccgggtgcgaggaggcgcgggggcagtgccagccgaagtctgccgttgcccaagaggcccaggcgtggcgctgcccctgagccggagcggacgcccgttgggcaggggtcctgggcccacccgggcaggacgcgtggaccgagtgaccgtggtttctgtgtggtgtcacctgccagacccgccgaagaagccacctctttggagggtgcgctctctggcacgcgccactcccacccatccgtgggccgccagcaccacgcgggccccccatccacatcgcggccaccacgtccctgggacacgccttgtcccccggtgtacgccgagaccaagcacttcctctactcctcaggcgacaaggagcagctgcggccctccttcctactcagctctctgaggcccagcctgactggcgctcggaggctcgtggagaccatctttctgggttccaggccctggatgccagggactccccgcaggttgccccgcctgccccagcgctactggcaaatgcggcccctgtttctggagctgcttgggaaccacgcgcagtgcccctacggggtgctcctcaagacgcactgcccgctgcgagctgcggtcaccccagcagccggtgtctgtgcccgggagaagccccagggctctgtggcggcccccgaggaggaggacacagacccccgtcgcctggtgcagctgctccgccagcacagcagcccctggcaggtgtacggcttcgtgcgggcctgcctgcgccggctggtgcccccaggcctctggggctccaggcacaacgaacgccgcttcctcaggaacaccaagaagttcatctccctggggaagcatgccaagctctcgctgcaggagctgacgtggaagatgagcgtgcgggactgcgcttggctgcgcaggagcccaggggttggctgtgttccggccgcagagcaccgtctgcgtgaggagatcctggccaagttcctgcactggctgatgagtgtgtacgtcgtcgagctgctcaggtctttcttttatgtcacggagaccacgtttcaaaagaacaggctctttttctaccggaagagtgtctggagcaagttgcaaagcattggaatcagacagcacttgaagagggtgcagctgcgggagctgtcggaagcagaggtcaggcagcatcgggaagccaggcccgccctgctgacgtccagactccgcttcatccccaagcctgacgggctgcggccgattgtgaacatggactacgtcgtgggagccagaacgttccgcagagaaaagagggccgagcgtctcacctcgagggtgaaggcactgttcagcgtgctcaactacgagcgggcgcggcgccccggcctcctgggcgcctctgtgctgggcctggacgatatccacagggcctggcgcaccttcgtgctgcgtgtgcgggcccaggacccgccgcctgagctgtactttgtcaaggtggatgtgacgggcgcgtacgacaccatcccccaggacaggctcacggaggtcatcgccagcatcatcaaaccccagaacacgtactgcgtgcgtcggtatgccgtggtccagaaggccgcccatgggcacgtccgcaaggccttcaagagccacgtctctaccttgacagacctccagccgtacatgcgacagttcgtggctcacctgcaggagaccagcccgctgagggatgccgtcgtcatcgagcagagctcctccctgaatgaggccagcagtggcctcttcgacgtcttcctacgcttcatgtgccaccacgccgtgcgcatcaggggcaagtcctacgtccagtgccaggggatcccgcagggctccatcctctccacgctgctctgcagcctgtgctacggcgacatggagaacaagctgtttgcggggattcggcgggacgggctgctcctgcgtttggtggatgatttcttgttggtgacacctcacctcacccacgcgaaaaccttcctcaggaccctggtccgaggtgtccctgagtatggctgcgtggtgaacttgcggaagacagtggtgaacttccctgtagaagacgaggccctgggtggcacggcttttgttcagatgccggcccacggcctattcccctggtgcggcctgctgctggatacccggaccctggaggtgcagagcgactactccagctatgcccggacctccatcagagccagtctcaccttcaaccgcggcttcaaggctgggaggaacatgcgtcgcaaactctttggggtcttgcggctgaagtgtcacagcctgtttctggatttgcaggtgaacagcctccagacggtgtgcaccaacatctacaagatcctcctgctgcaggcgtacaggtttcacgcatgtgtgctgcagctcccatttcatcagcaagtttggaagaaccccacatttttcctgcgcgtcatctctgacacggcctccctctgctactccatcctgaaagccaagaacgcagggatgtcgctgggggccaagggcgccgccggccctctgccctccgaggccgtgcagtggctgtgccaccaagcattcctgctcaagctgactcgacaccgtgtcacctacgtgccactcctggggtcactcaggacagcccagacgcagctgagtcggaagctcccggggacgacgctgactgccctggaggccgcagccaacccggcactgccctcagacttcaagaccatcctggactgatggccacccgcccacagccaggccgagagcagacaccagcagccctgtcacgccgggctctacgtcccagggagggaggggcggcccacacccaggcccgcaccgctgggagtctgaggcctgagtgagtgtttggccgaggcctgcatgtccggctgaaggctgagtgtccggctgaggcctgagcgagtgtccagccaagggctgagtgtccagcacacctgccgtcttcacttccccacaggctggcgctcggctccaccccagggccagcttttcctcaccaggagcccggcttccactccccacataggaatagtccatccccagattcgccattgttcacccctcgccctgccctcctttgccttccacccccaccatccaggtggagaccctgagaaggaccctgggagctctgggaatttggagtgaccaaaggtgtgccctgtacacaggcgaggaccctgcacctggatgggggtccctgtgggtcaaattggggggaggtgctgtgggagtaaaatactgaatatatgagtttttcagttttgaaaaaaa

SEQ ID NO: 18 - protein, 1132 aa, Homo sapiens
MPRAPRCRAVRSLLRSHYREVLPLATFVRRLGPQGWRLVQRGDPAAFRALVAQCLVCVPWDARPPPAAPSFRQVSCLKELVARVLQRLCERGAKNVLAFGFALLDGARGGPPEAFTTSVRSYLPNTVTDALRGSGAWGLLLRRVGDDVLVHLLARCALFVLVAPSCAYQVCGPPLYQLGAATQARPPPHASGPRRRLRCERAWNHSVREAGVPLGLPAPGARRRGGSASRSLPLPKRPRRGAAPEPERTPVGQGSWAHPGRTRGPSDRGFCVVSPARPAEEATSLEGALSGTRHSHPSVGRQHHAGPPSTSRPPRPWDTPCPPVYAETKHFLYSSGDKEQLRPSFLLSSLRPSLTGARRLVETIFLGSRPWMPGTPRRLPRLPQRYWQMRPLFLELLGNHAQCPYGVLLKTHCPLRAAVTPAAGVCAREKPQGSVAAPEEEDTDPRRLVQLLRQHSSPWQVYGFVRACLRRLVPPGLWGSRHNERRFLRNTKKFISLGKHAKLSLQELTWKMSVRDCAWLRRSPGVGCVPAAEHRLREEILAKFLHWLMSVYVVELLRSFFYVTETTFQKNRLFFYRKSVWSKLQSIGIRQHLKRVQLRELSEAEVRQHREARPALLTSRLRFIPKPDGLRPIVNMDYVVGARTFRREKRAERLTSRVKALFSVLNYERARRPGLLGASVLGLDDIHRAWRTFVLRVRAQDPPPELYFVKVDVTGAYDTIPQDRLTEVIASIIKPQNTYCVRRYAVVQKAAHGHVRKAFKSHVSTLTDLQPYMRQFVAHLQETSPLRDAVVIEQSSSLNEASSGLFDVFLRFMCHHAVRIRGKSYVQCQGIPQGSILSTLLCSLCYGDMENKLFAGIRRDGLLLRLVDDFLLVTPHLTHAKTFLRTLVRGVPEYGCVVNLRKTVVNFPVEDEALGGTAFVQMPAHGLFPWCGLLLDTRTLEVQSDYSSYARTSIRASLTFNRGFKAGRNMRRKLFGVLRLKCHSLFLDLQVNSLQTVCTNIYKILLLQAYRFHACVLQLPFHQQVWKNPTFFLRVISDTASLCYSILKAKNAGMSLGAKGAAGPLPSEAVQWLCHQAFLLKLTRHRVTYVPLLGSLRTAQTQLSRKLPGTTLTALEAAANPALPSDFKTILD

SEQ ID NO: 19 - protein, 1132 aa, Homo sapiens
MPRAPRCRAVRSLLRSHYREVLPLATFVRRLGPQGWRLVQRGDPAAFRALVAQCLVCVPWDARPPPAAPSFRQVSCLKELVARVLQRLCERGAKNVLAFGFALLDGARGGPPEAFTTSVRSYLPNTVTDALRGSGAWGLLLRRVGDDVLVHLLARCALFVLVAPSCAYQVCGPPLYQLGAATQARPPPHASGPRRRLGCERAWNHSVREAGVPLGLPAPGARRRGGSASRSLPLPKRPRRGAAPEPERTPVGQGSWAHPGRTRGPSDRGFCVVSPARPAEEATSLEGALSGTRHSHPSVGRQHHAGPPSTSRPPRPWDTPCPPVYAETKHFLYSSGDKEQLRPSFLLSSLRPSLTGARRLVETIFLGSRPWMPGTPRRLPRLPQRYWQMRPLFLELLGNHAQCPYGVLLKTHCPLRAAVTPAAGVCAREKPQGSVAAPEEEDTDPRRLVQLLRQHSSPWQVYGFVRACLRRLVPPGLWGSRHNERRFLRNTKKFISLGKHAKLSLQELTWKMSVRDCAWLRRSPGVGCVPAAEHRLREEILAKFLHWLMSVYVVELLRSFFYVTETTFQKNRLFFYRKSVWSKLQSIGIRQHLKRVQLRELSEAEVRQHREARPALLTSRLRFIPKPDGLRPIVNMDYVVGARTFRREKRAERLTSRVKALFSVLNYERARRPGLLGASVLGLDDIHRAWRTFVLRVRAQDPPPELYFVKVDVTGAYDTIPQDRLTEVIASIIKPQNTYCVRRYAVVQKAAHGHVRKAFKSHVSTLTDLQPYMRQFVAHLQETSPLRDAVVIEQSSSLNEASSGLFDVFLRFMCHHAVRIRGKSYVQCQGIPQGSILSTLLCSLCYGDMENKLFAGIRRDGLLLRLVDDFLLVTPHLTHAKTFLRTLVRGVPEYGCVVNLRKTVVNFPVEDEALGGTAFVQMPAHGLFPWCGLLLDTRTLEVQSDYSSYARTSIRASLTFNRGFKAGRNMRRKLFGVLRLKCHSLFLDLQVNSLQTVCTNIYKILLLQAYRFHACVLQLPFHQQVWKNPTFFLRVISDTASLCYSILKAKNAGMSLGAKGAAGPLPSEAVQWLCHQAFLLKLTRHRVTYVPLLGSLRTAQTQLSRKLPGTTLTALEAAANPALPSDFKTILD

SEQ ID NO: 20 - DNA, 2190 bp, Homo sapiens
atgcagctgtttgagcagccctgtcctggggaggacccccggccaggaggccagatcggtgaggtggagctgtcctcctacacgcccccagccggggtcccaggaaagcctgcagccccccacttccttccagtgctgtgctctgtgtcaccatcaggctccagggtcccgcacgacctcctcgggggctccgggggcttcacgctggaggacgccctcttcgggctcctctttggagctgatgccaccctcctgcagtcacctgtggtcctctgtggtctccctgatggccagctctgctgtgtgatcctgaaggccctggtcacctccaggtcagcccctggtgacccaaatgcccttgtcaagatcctccatcacctggaggagcccgtcatcttcataggggccttgaagacagagccacaggctgcagaagctgcagagaattttctgcctgacgaggatgtgcactgtgactgcctggtggcctttggtcaccacggccggatgctggccatcaaggccagctgggatgagtccgggaagctggtgcccgagctgcgggagtactgcctcccaggccctgtgctctgcgctgcctgtggcgggggtggccgcgtgtaccacagcaccccttctgacctctgtgtggtggatctgtctcggggaagcaccccgctgggccctgagcagcccgaagaaggcccgggaggcctgccccccatgctgtgcccagccagcctgaacatctgcagtgtcgtctcgctgtccgcgtctcccaggacgcatgaaggtggcaccaagctcctggccctgtccgccaaaggccgcctgatgacctgcagcctggacctggactctgagatgcctggcccagccaggatgaccacagagagtgcaggtcagaaaataaaggagctgctgtctggaattggcaacatctctgagagagtgtcttttctaaagaaggcggttgaccagcggaacaaggcactgacaagcctcaacgaggccatgaacgtgagctgtgcactgctgtcaagcggcacgggccccagacccatctcctgcaccaccagcaccacctggagccgcctgcagacacaggatgtgctcatggccacctgcgtgctagagaacagcagcagcttcagcctggaccaggggtggaccctgtgcatccaggtgctcaccagctcctgtgctctcgacctggactcggcctgctccgccatcacctacaccatccccgtggaccagctcggccccggtgctcggcgggaggtgacgctacccctgggccctggtgagaacggcgggctcgacctgcccgtgaccgtgtcctgcacgctgttctacagtctcagggaggtggtgggcggggcccttgcccccttagactctgaggacccctttctggatgagtgcccctccgacgtcctgcccgagcaagagggtgtttgcctgcccctgagcaggcacacagtggacatgctgcagtgtctgcgcttccctggcctggccccgccacacacacgggccccctccccactcggccccacccgagaccctgtggccacttttctggaaacttgtcgggagcctggcagccagccagcaggacccgcctccctgcgggccgagtacctgcccccatctgtggcttccatcaaggtgtcggcggagctgctcagagctgccttgaaggacggccactcaggcgtgcccctgtgctgtgccaccctgcagtggctccttgctgagaatgctgctgtggacgtcgtgagggcccgagcactatcttccatccagggagtggcccctgatggcgccaacgttcacctcatcgtccgagaggtggccatgaccgacctgtgcccagcagggcccatccaggccgtggagattcaagtggaaagctcctctctggccgacatttgcagggcgcaccatgccgttgtcgggcgcatgcagacgatggtgacagagcaggccgcccagggctccagcgctcctgatctccgtgtgcagtacctccgccagatccacgccaaccacgagacactgctgcgggaggtgcagaccctgcgcgaccggctctgcacggaggatgaggccagctcctgtgccaccgcccagaggctgctacaggtgtaccggcagctgcgccaccccagcctcatcctgctg

SEQ ID NO: 21 - DNA, 2190 bp, Homo sapiens
atgcagctgtttgagcagccctgtcctggggaggacccccggccaggaggccagatcggtgaggtggagctgtcctcctacacgcccccagccggggtcccaggaaagcctgcagccccccacttccttccagtgctgtgctctgtgtcaccatcaggctccagggtcccgcacgacctcctcgggggctccgggggcttcacgctggaggacgccctcttcgggctcctctttggagctgatgccaccctcctgcagtcacctgtggtcctctgtggtctccctgatggccagctctgctgtgtgatcctgaaggccctggtcacctccaggtcagcccctggtgacccaaatgcccttgtcaagatcctccatcacctggaggagcccgtcatcttcataggggccttgaagacagagccacaggctgcagaagctgcagagaattttctgcctgacgaggatgtgcactgtgactgcctggtggcctttggtcaccacggccggatgctggccatcaaggccagctgggatgagtccgggaagctggtgcccgagctgcgggagtactgcctcccaggccctgtgctctgcgctgcctgtggcgggggtggccgcgtgtaccacagcaccccttctgacctctgtgtggtggatctgtctcggggaagcaccccgctgggccctgagcagcccgaagaaggcccgggaggcctgccccccatgctgtgcccagccagcctgaacatctgcagtgtcgtctcgctgtccgcgtctcccaggacgcatgaaggtggcaccaagctcctggccctgtccgccaaaggccgcctgatgacctgcagcctggacctggactctgagatgcctggcccagccaggatgaccacagagagtgcaggtcagaaaataaaggagctgctgtctggaattggcaacatctctgagagagtgtcttttctaaagaaggcggttgaccagcggaacaaggcactgacaagcctcaacgaggccatgaacgtgagctgtgcactgctgtcaagcggcacgggccccagacccatctcctgcaccaccagcaccacctggagccgcctgcagacacaggatgtgctcatggccacctgcgtgctagagaacagcagcagcttcagcctggaccaggggtggaccctgtgcatccaggtgctcaccagctcctgtgctctcgacctggactcggcctgctccgccatcacctacaccatccccgtggaccagctcggccccggtgctcggcgggaggtgacgctacccctgggccctggtgagaacggcgggctcgacctgcccgtgaccgtgtcctgcacgctgttctacagtctcagggaggtggtgggcggggcccttgccccctcagactctgaggacccctttctggatgagtgcccctccgacgtcctgcccgagcaagagggtgtttgcctgcccctgagcaggcacacagtggacatgctgcagtgtctgcgcttccctggcctggccccgccacacacacgggccccctccccactcggccccacccgagaccctgtggccacttttctggaaacttgtcgggagcctggcagccagccagcaggacccgcctccctgcgggccgagtacctgcccccatctgtggcttccatcaaggtgtcggcggagctgctcagagctgccttgaaggacggccactcaggcgtgcccctgtgctgtgccaccctgcagtggctccttgctgagaatgctgctgtggacgtcgtgagggcccgagcactatcttccatccagggagtggcccctgatggcgccaacgttcacctcatcgtccgagaggtggccatgaccgacctgtgcccagcagggcccatccaggccgtggagattcaagtggaaagctcctctctggccgacatttgcagggcgcaccatgccgttgtcgggcgcatgcagacgatggtgacagagcaggccgcccagggctccagcgctcctgatctccgtgtgcagtacctccgccagatccacgccaaccacgagacactgctgcgggaggtgcagaccctgcgcgaccggctctgcacggaggatgaggccagctcctgtgccaccgcccagaggctgctacaggtgtaccggcagctgcgccaccccagcctcatcctgctg

SEQ ID NO: 22 - DNA, 3163 bp, Homo sapiens
ggacagtgtgctggtcaccctggtgcagggccctgcccgatggaagatgcagctgtttgagcagccctgtcctggggaggacccccggccaggaggccagatcggtgaggtggagctgtcctcctacacgcccccagccggggtcccaggaaagcctgcagccccccacttccttccagtgctgtgctctgtgtcaccatcaggctccagggtcccgcacgacctcctcgggggctccgggggcttcacgctggaggacgccctcttcgggctcctctttggagctgatgccaccctcctgcagtcacctgtggtcctctgtggtctccctgatggccagctctgctgtgtgatcctgaaggccctggtcacctccaggtcagcccctggtgacccaaatgcccttgtcaagatcctccatcacctggaggagcccgtcatcttcataggggccttgaagacagagccacaggctgcagaagctgcagagaattttctgcctgacgaggatgtgcactgtgactgcctggtggcctttggtcaccacggccggatgctggccatcaaggccagctgggatgagtccgggaagctggtgcccgagctgcgggagtactgcctcccaggccctgtgctctgcgctgcctgtggcgggggtggccgcgtgtaccacagcaccccttctgacctctgtgtggtggatctgtctcggggaagcaccccgctgggccctgagcagcccgaagaaggcccgggaggcctgccccccatgctgtgcccagccagcctgaacatctgcagtgtcgtctcgctgtccgcgtctcccaggacgcatgaaggtggcaccaagctcctggccctgtccgccaaaggccgcctgatgacctgcagcctggacctggactctgagatgcctggcccagccaggatgaccacagagagtgcaggtcagaaaataaaggagctgctgtctggaattggcaacatctctgagagagtgtcttttctaaagaaggcggttgaccagcggaacaaggcactgacaagcctcaacgaggccatgaacgtgagctgtgcactgctgtcaagcggcacgggccccagacccatctcctgcaccaccagcaccacctggagccgcctgcagacacaggatgtgctcatggccacctgcgtgctagagaacagcagcagcttcagcctggaccaggggtggaccctgtgcatccaggtgctcaccagctcctgtgctctcgacctggactcggcctgctccgccatcacctacaccatccccgtggaccagctcggccccggtgctcggcgggaggtgacgctacccctgggccctggtgagaacggcgggctcgacctgcccgtgaccgtgtcctgcacgctgttctacagtctcagggaggtggtgggcggggcccttgccccctcagactctgaggacccctttctggatgagtgcccctccgacgtcctgcccgagcaagagggtgtttgcctgcccctgagcaggcacacagtggacatgctgcagtgtctgcgcttccctggcctggccccgccacacacacgggccccctccccactcggccccacccgagaccctgtggccacttttctggaaacttgtcgggagcctggcagccagccagcaggacccgcctccctgcgggccgagtacctgcccccatctgtggcttccatcaaggtgtcggcggagctgctcagagctgccttgaaggacggccactcaggcgtgcccctgtgctgtgccaccctgcagtggctccttgctgagaatgctgctgtggacgtcgtgagggcccgagcactatcttccatccagggagtggcccctgatggcgccaacgttcacctcatcgtccgagaggtggccatgaccgacctgtgcccagcagggcccatccaggccgtggagattcaagtggaaagctcctctctggccgacatttgcagggcgcaccatgccgttgtcgggcgcatgcagacgatggtgacagagcaggccgcccagggctccagcgctcctgatctccgtgtgcagtacctccgccagatccacgccaaccacgagacactgctgcgggaggtgcagaccctgcgcgaccggctctgcacggaggatgaggccagctcctgtgccaccgcccagaggctgctacaggtgtaccggcagctgcgccaccccagcctcatcctgctgtgaccaggcgggcctgcccctgggctctggccacgcttccagcctctgtcacagcccccccaggcctcatgggttagagggaaaccgagctggcctggccagagccgtcagggaaggtaggacctggccacgtaggagcagaacgctcatgaaagtgcttggaggccgtggagcacaaagcagattctgattgggagcaaccgaggcgggctctgaacctggccggtccagcttcgcgtcctctgctggtgtctctccttctctgaccgcggccgcagcccctgcactcgccttcctcactgctgggcagccttcccaccaccgcagcagcccctgaggccaggaggcagtgcagggcattctggacccggagggccagagaaacaggatttctggggtttggacttggggtgagtttgtaactgttgctgccacaccaccaggagcaccggctgcccctctgggtggcactaccaggtgccccacggtacccttgtcacactgttcacacctgcccggctgcccactctgggaccccgaggtaggagggtgctccctgagaccaaagcacaaaacagcatgcagggagctcctgcaagtgcccgtggtctcgtgccacaccaaggaagggccagcgggtggcctgtggccggaatgctcaacaactaggtgcctccggccggggcagtacccagcactgtgcactattttcagggccactcagggtggcgctgtggcccgggggggggccctgagccccagcccccagcctcctccctcagcctgggctacggcccacctcctggtgctggtgttttcatctggggagggtgctcgcgccgctcccgctgcaggcactgtccgcgatgagtgcgggtaggagccgtgaggtgcttctctgctgtgacaaacgaccctgtctgtccg

SEQ ID NO: 23 - protein, 730 aa, Homo sapiens
MQLFEQPCPGEDPRPGGQIGEVELSSYTPPAGVPGKPAAPHFLPVLCSVSPSGSRVPHDLLGGSGGFTLEDALFGLLFGADATLLQSPVVLCGLPDGQLCCVILKALVTSRSAPGDPNALVKILHHLEEPVIFIGALKTEPQAAEAAENFLPDEDVHCDCLVAFGHHGRMLAIKASWDESGKLVPELREYCLPGPVLCAACGGGGRVYHSTPSDLCVVDLSRGSTPLGPEQPEEGPGGLPPMLCPASLNICSVVSLSASPRTHEGGTKLLALSAKGRLMTCSLDLDSEMPGPARMTTESAGQKIKELLSGIGNISERVSFLKKAVDQRNKALTSLNEAMNVSCALLSSGTGPRPISCTTSTTWSRLQTQDVLMATCVLENSSSFSLDQGWTLCIQVLTSSCALDLDSACSAITYTIPVDQLGPGARREVTLPLGPGENGGLDLPVTVSCTLFYSLREVVGGALAPLDSEDPFLDECPSDVLPEQEGVCLPLSRHTVDMLQCLRFPGLAPPHTRAPSPLGPTRDPVATFLETCREPGSQPAGPASLRAEYLPPSVASIKVSAELLRAALKDGHSGVPLCCATLQWLLAENAAVDVVRARALSSIQGVAPDGANVHLIVREVAMTDLCPAGPIQAVEIQVESSSLADICRAHHAVVGRMQTMVTEQAAQGSSAPDLRVQYLRQIHANHETLLREVQTLRDRLCTEDEASSCATAQRLLQVYRQLRHPSLILL

SEQ ID NO: 24 - protein, 730 aa, Homo sapiens [sequence omitted]

SEQ ID NO: 25 - DNA, 6946 bp, Homo sapiens
atgagcagccgacggaaacgtgctcctccagtgagggtagatgaggaaaagaggcagcagcttcattggaatatgcatgaggacagaaggaatgaacctatcatcataagtgatgatgacgagcagccctgcccaggttcagatacctcttctgctcattatatcattctaagtgatagtctaaaggaagaagtggctcacagagataagaagaggtgttcaaaagtggtgagcttctcaaaaccaattgaaaaagaagagactgttggtattttttcccctttgtctgtaaagttaaatattgtgatttctccctatcattttgataattcctggaaagcatttctaggagaattaactcttcagcttcttcctgcacagagtttaattgaaaatttttccgaaaggagtattacattgatgagttcagagtcaagcaatcagttcctgatttatgttcattcaaaaggtgaagatgtagagaaacaaaaaaaagaaccgatgagtatttgtgacaagggtattctggtggagtcatccttcagtggtgaaatgttagaagatttggggtggctacaaaagaagagaagaataaaactctatcagaaaccagaaggaaatcacattatcaaggttggaatttatcttttggaagctggcctagcaaaactagacttcttgagtgatgcaaattcaagaatgaaaaagttcaatcagctcatgaagaaagtaatggaaaagttacacaattctattattccagatgtgttggaagaggatgaagatgatccggagagtgagccagagggacaagacattgacgagctgtatcactttgtgaaacaaacacatcagcaagaaacgcagtccatccaagtggatgtccagcatcctgcattgatccctgtgttgagaccctaccaaagagaggctgtcaattggatgctacaacaagagtgtttcagaagcagtcctgctactgaaagtgccctgcacttcttatggcgagagattgttacatctgagggtctgaaactctactataatccatatacaggctgcatcattcgtgagtacccaaattctgggccgcagttgcttggtggaattttagcagatgagatgggtcttggaaagacagtggaggttttggctctgattctgacacatactcgacaagatgtcaagcaagatgctctcactcttcccgagggaaaagtggtgaattattttattccgtcacattattttggaggaaaactgaaaaagacagaaatccagaatatcgaatttgaaccaaaagaaaaagttcaatgccctcctacacgtgtgatgatactgacagctgtgaaagaaatgaatggaaaaaaaggagtgtccatcctttccatctataagtatgtcagttctatatatagatacgatgttcaacggaacaggagtcttttgaaacggatgctgaaatgtttaatttttgaaggtctcgtgaaacagatcaaaggccatggtttttctgggacttttacattgggaaagaattacaaggaagaggatatatgtgataaaacaaaaaagcaggcagtagggagtccaaggaaaattcagaaagaaactagaaaatcagggaacaaggacacagattctgaatacttgccatcagacacctctgatgatgatgatgatccttactattattattataagtccaggagaaatcgcagtaaattgaggaaaaagcttgttccatccacaaaaaaaggaaaaagtcaaccatttatcaatcccgattcacaaggtcactgtccagctactagcgactctggaataactgatgttgctatgtctaaaagtacatgtatctctgaattcaaccaagaacatgaaacagaggactgtgctgaatctctaaatcatgctgatagtgatgtaccaccttctaataccatgagtccctttaacacctctgattaccgctttgagtgtatatgtggtgaacttgatcagatagatcgtaagcctcgtgttcaatgcctgaagtgtcacctgtggcaacatgcaaagtgtgtgaattatgatgagaaaaatctgaagatcaagcctttttactgcccccactgccttgttgcaatggaaccagtgtcaacaagagcaactctgatcatctctccaagttccatctgtcaccagtgggtggatgagatcaacaggcatgtgaggtcgtcatctcttcgagtcttggtatatcaaggagtgaagaaagatggctttttacaacctcattttttggcagaacaggatatagttatcattacctatgatgtactgcgttcagaattaaattatgtcgatatcccacatagcaatagtgaggatgggcgtcgcctacggaaccagaagcgctatatggctatcccgagccccctggtagctgtggagtggtggaggatctgccttgatgaagctcagatggttgagtgtcccacagtaaaagctgcagaaatggcccagcgtttgagtgggattaatcgatggtgtatcagtggcactccagtacagagaggattagaggatctttttgggttagtggtctttcttggtattgaaccttactgtgtcaaacactggtgggttcgacttctctatcggccttactgcaagaagaatcctcagcatctctacagctttattgccaagatactgtggaggtctgcaaagaaagatgtgattgaccaaatccaaataccaccacaaaccgaagaaatacactggctccacttttctccagtggaaaggcatttctatcaccgtcagcatgaggtgtgctgccaggatgtggtggtaaaactcaggaagatttctgactgggctctgaagctcagcagcctagacagaaggactgtcacctctatcctgtatccattgctgaggctcagacaggcctgctgtcacccacaggctgttcgtggagagttcttgccactccaaaaaagcaccatgacaatggaagagctgctgacatctttgcagaagaaatgtggaactgaatgtgaagaagcacatcgacagctagtttgtgctctcaatggcttagcaggcattcatattattaaaggtgagtatgccttggcagcagaattgtacagagaagtgttgcgctcctcggaggaacacaaaggaaaactcaaaactgattcacttcaaagacttcatgctacccataacttgatggaattgttgatagccaggcacccagggataccacctaccttgcgtgatggccgacttgaggaagaggccaaacagctgcgagagcactacatgagcaagtgtaatacagaagttgctgaagcccagcaagctttatatcctgtgcagcagaccatccatgagcttcaaagaaagattcattctaattctccttggtggctaaatgtgatccacagagcaatagaatttactattgatgaggagctagttcagcgagtgcgaaatgaaataaccagcaactacaagcaacaaactggcaagctttctatgtcagagaagttctgtgattgcagaggtcttcagttcttacttacaacacaaatggaagagctaaataaatgccagaagctagtaagagaggctgtaaaaaacctggagggacctccatctcgtaatgttattgagtctgcaacagtctgtcacctccgaccagccagacttcctctcaactgctgtgtcttttgtaaagctgatgaattgttcacagagtatgaatcaaagctattttccaacacagtcaaaggccagactgcaatatttgaggagatgatagaagatgaagaaggactggtggatgatcgagcacctaccaccacccggggtctctgggcaataagtgagacagagcgatctatgaaagcaatactatcatttgcaaaatcacataggtttgatgttgaatttgttgatgaaggaagcacttcaatggatctctttgaagcatggaagaaggaatataagttgcttcatgaatattggatggctctgaggaatcgtgtgtctgctgttgatgaacttgcaatggctacagaacgactaagagtgcgtgatcctagggagccaaagcctaatccgcctgttcttcatatcattgaaccacatgaggtagaacaaaaccgaataaaactactaaatgataaagctgttgctacatcacagcttcagaaaaaacttgggcagcttctttacctaactaatttggagaagtctcaagataaaacatcgggaggtgttaatccagaaccttgcccaatctgtgctcgacagctaggaaaacagtgggcggtactgacctgtggtcactgtttctgtaatgaatgcatttctataattattgaacaatacagcgtgggatctcacagaagctccattaagtgtgcaatctgccgccagaccacatctcacaaagaaatctcgtatgtctttacctcagagaaagcaaaccaggaggaggacatccctgtgaagggcagccattctacaaaagtggaagctgtggtcagaactctgatgaaaatacagcttagagatccaggggccaaagcactcgttttctcaacgtggcaagatgtattagatattatttcaaaagctcttactgacaacaacatggaatttgcacaaatcagtcgtgttaagacatttcaggagaacctttcagcatttaaacgtgatccccaaatcaatattttgctgctgcccctgcacacaggttctaatggattaactatcattgaagcaactcatgttctcttggtggagcccatattgaaccctgcccatgagcttcaggccatagggagggtgcaccgaattggacagacaaaacctactattgtacacagattcttaattaaagcaacaatagaagaaagaatgcaggcaatgctgaaaactgctgagagaagtcacacgaactcatcagcaaagcattcagaggcctctgtcttgactgtggctgacctggcagacctatttaccaaagaaactgaagagcttgaatgaactacacttgattcattccatggactttagtgtattaataaactttcatagctgtagagcaaagttacaagttttaaaaacccagtagataacagtaacactgttcctagttgaatgaatttgtttattgttctggttatacagtggcttagtacttattgagtccttttcaatatactgtttccaaaagatctataaagattcttaattttacactcctaataaaacatttattttttgtcccaaatagtatctatatctgatgatgcaagggaattaagagaggtaatgtgtacttttcttatgataagatcactcataaaacagttggaataagaaagtaaactagtctagttttatatgacctggtatattgaacaaacatagatctcagttttggaagggccttcagtatctttagcacatgattcaccaatatcttgttagtaatattttaagtagatggtgaattatattaacccattttttgagtaggcttataccataactttacacctagtaattttgcagtaatacaactccttacaataagttaatgttagacagttatttgtttcttctgcatcttgctactgaaaacaaaaacaatgaaaatattcatatttctattatgtgtaaagtggtgttagtgtaaacagttaactccaaatagattttttaatgtattaatgattaccttaaaattatctgctttccctactcttaatttttcttattgttaaagtttttatatgttgtaagattgatgtatctgtaaatattgcagtttttatgattttgttttattataacatggaaatatggcagggttttttttttaatcattattatactgatgtcttggattctttttctttcccatgttcttgtgtaactagactttttagccagtgatatctgaattcatggtagagttgttatttcactccagttgttcctcccttaagcacgagtctttgaccttttgaacaagagaatgccatgttcacataagcaaatcgttgcaagtgataataacgaagtgtttcttcttctgtgaggaattaatggactttgaaattgctgaataaggagtagcttgttacatcctagccatttgatgtaatacaaatgttcttatttggcttccttttctttgagccttcagagttttaaaggaccctctttgaccctcaggacttattagctcccagttagctgcctggaaaactcatagaaccattccctagatcccatttgtccgagaggctttggtgcttagttaaggtgcattttcttactctaggtttatagagtgatgtaattcaactgaagaaaacacacacatgcttttggtgcttattcttcagttaaaaggaggtatccattgattcttttatatgaaattaaatcaaactgttatttttatagctcttcacatattaaaaagagctttcatgtacatttacgtgtttaatttacccaacaatcttgtgaagttgatataattaatatctttactgttttaaaggtagagaactgaagttcagatatattgaaactttcttaggacatgtagcaagtaaacagtggagcccaggtcccctgactccatatcttacattcttttactaataacatgtcatgctgagattcagaaggcccatgcttataatttaaaaatgagactatttcagtatttctttgtgtttgttttctactattcctttctgagctatgttttagagaaaacaatttgatatgcttcaattttgtaaaatcaaattacatatatataaaaaaacctatctgtattagacaaaaactgtttttatttatttctgtaagatatccattaaataaatattttagtggaaatgaaaaaaaaaaaaaa

SEQ ID NO: 26 - DNA, 6946 bp, Homo sapiens
atgagcagccgacggaaacgtgctcctccagtgagggtagatgaggaaaagaggcagcagcttcattggaatatgcatgaggacagaaggaatgaacctatcatcataagtgatgatgacgagcagccctgcccaggttcagatacctcttctgctcattatatcattctaagtgatagtctaaaggaagaagtggctcacagagataagaagaggtgttcaaaagtggtgagcttctcaaaaccaattgaaaaagaagagactgttggtattttttcccctttgtctgtaaagttaaatattgtgatttctccctatcattttgataattcctggaaagcatttctaggagaattaactcttcagcttcttcctgcacagagtttaattgaaaatttttccgaaaggagtattacattgatgagttcagagtcaagcaatcagttcctgatttatgttcattcaaaaggtgaagatgtagagaaacaaaaaaaagaaccgatgagtatttgtgacaagggtattctggtggagtcatccttcagtggtgaaatgttagaagatttggggtggctacaaaagaagagaagaataaaactctatcagaaaccagaaggaaatcacattatcaaggttggaatttatcttttggaagctggcctagcaaaactagacttcttgagtgatgcaaattcaagaatgaaaaagttcaatcagctcatgaagaaagtaatggaaaagttacacaattctattattccagatgtgttggaagaggatgaagatgatccggagagtgagccagagggacaagacattgacgagctgtatcactttgtgaaacaaacacatcagcaagaaacgcagtccatccaagtggatgtccagcatcctgcattgatccctgtgttgagaccctaccaaagagaggctgtcaattggatgctacaacaagagtgtttcagaagcagtcctgctactgaaagtgccctgcacttcttatggcgagagattgttacatctgagggtctgaaactctactataatccatatacaggctgcatcattcgtgagtacccaaattctgggccgcagttgcttggtggaattttagcagatgagatgggtcttggaaagacagtggaggttttggctctgattctgacacatactcgacaagatgtcaagcaagatgctctcactcttcccgagggaaaagtggtgaattattttattccgtcacattattttggaggaaaactgaaaaagacagaaatccagaatatcgaatttgaaccaaaagaaaaagttcaatgccctcctacacgtgtgatgatactgacagctgtgaaagaaatgaatggaaaaaaaggagtgtccatcctttccatctataagtatgtcagttctatatatagatacgatgttcaacggaacaggagtcttttgaaacggatgctgaaatgtttaatttttgaaggtctcgtgaaacagatcaaaggccatggtttttctgggacttttacattgggaaagaattacaaggaagaggatatatgtgataaaacaaaaaagcaggcagtagggagtccaaggaaaattcagaaagaaactagaaaatcagggaacaaggacacagattctgaatacttgccatcagacacctctgatgatgatgatgatccttactattattattataagtccaggagaaatcgcagtaaattgaggaaaaagcttgttccatccacaaaaaaaggaaaaagtcaaccatttatcaatcccgattcacaaggtcactgtccagctactagcgactctggaataactgatgttgctatgtctaaaagtacatgtatctctgaattcaaccaagaacatgaaacagaggactgtgctgaatctctaaatcatgctgatagtgatgtaccaccttctaataccatgagtccctttaacacctctgattaccgctttgagtgtatatgtggtgaacttgatcagatagatcgtaagcctcgtgttcaatgcctgaagtgtcacctgtggcaacatgcaaagtgtgtgaattatgatgagaaaaatctgaagatcaagcctttttactgcccccactgccttgttgcaatggaaccagtgtcaacaagagcaactctgatcatctctccaagttccatctgtcaccagtgggtggatgagatcaacaggcatgtgaggtcgtcatctcttcgagtcttggtatatcaaggagtgaagaaagatggctttttacaacctcattttttggcagaacaggatatagttatcattacctatgatgtactgcgttcagaattaaattatgtcgatatcccacatagcaatagtgaggatgggcgtcgcctacggaaccagaagcgctatatggctatcccgagccccctggtagctgtggagtggtggaggatctgccttgatgaagctcagatggttgagtgtcccacagtaaaagctgcagaaatggcccagcgtttgagtgggattaatcgatggtgtatcagtggcactccagtacagagaggattagaggatctttttgggttagtggtctttcttggtattgaaccttactgtgtcaaacactggtgggttcgacttctctatcggccttactgcaagaagaatcctcagcatctctacagctttattgccaagatactgtggaggtctgcaaagaaagatgtgattgaccaaatccaaataccaccacaaaccgaagaaatacactggctccacttttctccagtggaaaggcatttctatcaccgtcagcatgaggtgtgctgccaggatgtggtggtaaaactcaggaagatttctgactgggctctgaagctcagcagcctagacagaaggactgtcacctctatcctgtatccattgctgaggctcagacaggcctgctgtcacccacaggctgttcgtggagagttcttgccactccaaaaaagcaccatgacaatggaagagctgctgacatctttgcagaagaaatgtggaactgaatgtgaagaagcacatcgacagctagtttgtgctctcaatggcttagcaggcattcatattattaaaggtgagtatgccttggcagcagaattgtacagagaagtgttgcgctcctcggaggaacacaaaggaaaactcaaaactgattcacttcaaagacttcatgctacccataacttgatggaattgttgatagccaggcacccagggataccacctaccttgcgtgatggccgacttgaggaagaggccaaacagctgcgagagcactacatgagcaagtgtaatacagaagttgctgaagcccagcaagctttatatcctgtgcagcagaccatccatgagcttcaaagaaagattcattctaattctccttggtggctaaatgtgatccacagagcaatagaatttactattgatgaggagctagttcagcgagtgcgaaatgaaataaccagcaactacaagcaacaaactggcaagctttctatgtcagagaagttccgtgattgcagaggtcttcagttcttacttacaacacaaatggaagagctaaataaatgccagaagctagtaagagaggctgtaaaaaacctggagggacctccatctcgtaatgttattgagtctgcaacagtctgtcacctccgaccagccagacttcctctcaactgctgtgtcttttgtaaagctgatgaattgttcacagagtatgaatcaaagctattttccaacacagtcaaaggccagactgcaatatttgaggagatgatagaagatgaagaaggactggtggatgatcgagcacctaccaccacccggggtctctgggcaataagtgagacagagcgatctatgaaagcaatactatcatttgcaaaatcacataggtttgatgttgaatttgttgatgaaggaagcacttcaatggatctctttgaagcatggaagaaggaatataagttgcttcatgaatattggatggctctgaggaatcgtgtgtctgctgttgatgaacttgcaatggctacagaacgactaagagtgcgtgatcctagggagccaaagcctaatccgcctgttcttcatatcattgaaccacatgaggtagaacaaaaccgaataaaactactaaatgataaagctgttgctacatcacagcttcagaaaaaacttgggcagcttctttacctaactaatttggagaagtctcaagataaaacatcgggaggtgttaatccagaaccttgcccaatctgtgctcgacagctaggaaaacagtgggcggtactgacctgtggtcactgtttctgtaatgaatgcatttctataattattgaacaatacagcgtgggatctcacagaagctccattaagtgtgcaatctgccgccagaccacatctcacaaagaaatctcgtatgtctttacctcagagaaagcaaaccaggaggaggacatccctgtgaagggcagccattctacaaaagtggaagctgtggtcagaactctgatgaaaatacagcttagagatccaggggccaaagcactcgttttctcaacgtggcaagatgtattagatattatttcaaaagctcttactgacaacaacatggaatttgcacaaatcagtcgtgttaagacatttcaggagaacctttcagcatttaaacgtgatccccaaatcaatattttgctgctgcccctgcacacaggttctaatggattaactatcattgaagcaactcatgttctcttggtggagcccatattgaaccctgcccatgagcttcaggccatagggagggtgcaccgaattggacagacaaaacctactattgtacacagattcttaattaaagcaacaatagaagaaagaatgcaggcaatgctgaaaactgctgagagaagtcacacgaactcatcagcaaagcattcagaggcctctgtcttgactgtggctgacctggcagacctatttaccaaagaaactgaagagcttgaatgaactacacttgattcattccatggactttagtgtattaataaactttcatagctgtagagcaaagttacaagttttaaaaacccagtagataacagtaacactgttcctagttgaatgaatttgtttattgttctggttatacagtggcttagtacttattgagtccttttcaatatactgtttccaaaagatctataaagattcttaattttacactcctaataaaacatttattttttgtcccaaatagtatctatatctgatgatgcaagggaattaagagaggtaatgtgtacttttcttatgataagatcactcataaaacagttggaataagaaagtaaactagtctagttttatatgacctggtatattgaacaaacatagatctcagttttggaagggccttcagtatctttagcacatgattcaccaatatcttgttagtaatattttaagtagatggtgaattatattaacccattttttgagtaggcttataccataactttacacctagtaattttgcagtaatacaactccttacaataagttaatgttagacagttatttgtttcttctgcatcttgctactgaaaacaaaaacaatgaaaatattcatatttctattatgtgtaaagtggtgttagtgtaaacagttaactccaaatagattttttaatgtattaatgattaccttaaaattatctgctttccctactcttaatttttcttattgttaaagtttttatatgttgtaagattgatgtatctgtaaatattgcagtttttatgattttgttttattataacatggaaatatggcagggttttttttttaatcattattatactgatgtcttggattctttttctttcccatgttcttgtgtaactagactttttagccagtgatatctgaattcatggtagagttgttatttcactccagttgttcctcccttaagcacgagtctttgaccttttgaacaagagaatgccatgttcacataagcaaatcgttgcaagtgataataacgaagtgtttcttcttctgtgaggaattaatggactttgaaattgctgaataaggagtagcttgttacatcctagccatttgatgtaatacaaatgttcttatttggcttccttttctttgagccttcagagttttaaaggaccctctttgaccctcaggacttattagctcccagttagctgcctggaaaactcatagaaccattccctagatcccatttgtccgagaggctttggtgcttagttaaggtgcattttcttactctaggtttatagagtgatgtaattcaactgaagaaaacacacacatgcttttggtgcttattcttcagttaaaaggaggtatccattgattcttttatatgaaattaaatcaaactgttatttttatagctcttcacatattaaaaagagctttcatgtacatttacgtgtttaatttacccaacaatcttgtgaagttgatataattaatatctttactgttttaaaggtagagaactgaagttcagatatattgaaactttcttaggacatgtagcaagtaaacagtggagcccaggtcccctgactccatatcttacattcttttactaataacatgtcatgctgagattcagaaggcccatgcttataatttaaaaatgagactatttcagtatttctttgtgtttgttttctactattcctttctgagctatgttttagagaaaacaatttgatatgcttcaattttgtaaaatcaaattacatatatataaaaaaacctatctgtattagacaaaaactgtttttatttatttctgtaagatatccattaaataaatattttagtggaaatgaaaaaaaaaaaaaa

SEQ ID NO: 27 - DNA, 7345 bp, Homo sapiens
gtctttggcctccgggaccagggagggaggacgctggttgtcctggagcccgggaagctcaagggctgagagggagccgctgcgcgagtcttccccacctcttcccgatcccggactagcgaacttcggaccaccacgtccctccggggataaagtggggtcgtgggaaaggaggctgtgcttctcctcatgcactggggcctgcggtgtttgtctcgagcgtccaacacactaggatccgggagtccgtcgtcacgctccttggcctcgcggtcgcttgtttgctcggcctgctgggagcgatccggcgctgacagccccgctcagactccgggagccgcttgtgggtctttcggaagataactgaagttcacagttaccagccaagaaaacttgaaaatgagcagccgacggaaacgtgctcctccagtgagggtagatgaggaaaagaggcagcagcttcattggaatatgcatgaggacagaaggaatgaacctatcatcataagtgatgatgacgagcagccctgcccaggttcagatacctcttctgctcattatatcattctaagtgatagtctaaaggaagaagtggctcacagagataagaagaggtgttcaaaagtggtgagcttctcaaaaccaattgaaaaagaagagactgttggtattttttcccctttgtctgtaaagttaaatattgtgatttctccctatcattttgataattcctggaaagcatttctaggagaattaactcttcagcttcttcctgcacagagtttaattgaaaatttttccgaaaggagtattacattgatgagttcagagtcaagcaatcagttcctgatttatgttcattcaaaaggtgaagatgtagagaaacaaaaaaaagaaccgatgagtatttgtgacaagggtattctggtggagtcatccttcagtggtgaaatgttagaagatttggggtggctacaaaagaagagaagaataaaactctatcagaaaccagaaggaaatcacattatcaaggttggaatttatcttttggaagctggcctagcaaaactagacttcttgagtgatgcaaattcaagaatgaaaaagttcaatcagctcatgaagaaagtaatggaaaagttacacaattctattattccagatgtgttggaagaggatgaagatgatccggagagtgagccagagggacaagacattgacgagctgtatcactttgtgaaacaaacacatcagcaagaaacgcagtccatccaagtggatgtccagcatcctgcattgatccctgtgttgagaccctaccaaagagaggctgtcaattggatgctacaacaagagtgtttcagaagcagtcctgctactgaaagtgccctgcacttcttatggcgagagattgttacatctgagggtctgaaactctactataatccatatacaggctgcatcattcgtgagtacccaaattctgggccgcagttgcttggtggaattttagcagatgagatgggtcttggaaagacagtggaggttttggctctgattctgacacatactcgacaagatgtcaagcaagatgctctcactcttcccgagggaaaagtggtgaattattttattccgtcacattattttggaggaaaactgaaaaagacagaaatccagaatatcgaatttgaaccaaaagaaaaagttcaatgccctcctacacgtgtgatgatactgacagctgtgaaagaaatgaatggaaaaaaaggagtgtccatcctttccatctataagtatgtcagttctatatatagatacgatgttcaacggaacaggagtcttttgaaacggatgctgaaatgtttaatttttgaaggtctcgtgaaacagatcaaaggccatggtttttctgggacttttacattgggaaagaattacaaggaagaggatatatgtgataaaacaaaaaagcaggcagtagggagtccaaggaaaattcagaaagaaactagaaaatcagggaacaaggacacagattctgaatacttgccatcagacacctctgatgatgatgatgatccttactattattattataagtccaggagaaatcgcagtaaattgaggaaaaagcttgttccatccacaaaaaaaggaaaaagtcaaccatttatcaatcccgattcacaaggtcactgtccagctactagcgactctggaataactgatgttgctatgtctaaaagtacatgtatctctgaattcaaccaagaacatgaaacagaggactgtgctgaatctctaaatcatgctgatagtgatgtaccaccttctaataccatgagtccctttaacacctctgattaccgctttgagtgtatatgtggtgaacttgatcagatagatcgtaagcctcgtgttcaatgcctgaagtgtcacctgtggcaacatgcaaagtgtgtgaattatgatgagaaaaatctgaagatcaagcctttttactgcccccactgccttgttgcaatggaaccagtgtcaacaagagcaactctgatcatctctccaagttccatctgtcaccagtgggtggatgagatcaacaggcatgtgaggtcgtcatctcttcgagtcttggtatatcaaggagtgaagaaagatggctttttacaacctcattttttggcagaacaggatatagttatcattacctatgatgtactgcgttcagaattaaattatgtcgatatcccacatagcaatagtgaggatgggcgtcgcctacggaaccagaagcgctatatggctatcccgagccccctggtagctgtggagtggtggaggatctgccttgatgaagctcagatggttgagtgtcccacagtaaaagctgcagaaatggcccagcgtttgagtgggattaatcgatggtgtatcagtggcactccagtacagagaggattagaggatctttttgggttagtggtctttcttggtattgaaccttactgtgtcaaacactggtgggttcgacttctctatcggccttactgcaagaagaatcctcagcatctctacagctttattgccaagatactgtggaggtctgcaaagaaagatgtgattgaccaaatccaaataccaccacaaaccgaagaaatacactggctccacttttctccagtggaaaggcatttctatcaccgtcagcatgaggtgtgctgccaggatgtggtggtaaaactcaggaagatttctgactgggctctgaagctcagcagcctagacagaaggactgtcacctctatcctgtatccattgctgaggctcagacaggcctgctgtcacccacaggctgttcgtggagagttcttgccactccaaaaaagcaccatgacaatggaagagctgctgacatctttgcagaagaaatgtggaactgaatgtgaagaagcacatcgacagctagtttgtgctctcaatggcttagcaggcattcatattattaaaggtgagtatgccttggcagcagaattgtacagagaagtgttgcgctcctcggaggaacacaaaggaaaactcaaaactgattcacttcaaagacttcatgctacccataacttgatggaattgttgatagccaggcacccagggataccacctaccttgcgtgatggccgacttgaggaagaggccaaacagctgcgagagcactacatgagcaagtgtaatacagaagttgctgaagcccagcaagctttatatcctgtgcagcagaccatccatgagcttcaaagaaagattcattctaattctccttggtggctaaatgtgatccacagagcaatagaatttactattgatgaggagctagttcagcgagtgcgaaatgaaataaccagcaactacaagcaacaaactggcaagctttctatgtcagagaagttccgtgattgcagaggtcttcagttcttacttacaacacaaatggaagagctaaataaatgccagaagctagtaagagaggctgtaaaaaacctggagggacctccatctcgtaatgttattgagtctgcaacagtctgtcacctccgaccagccagacttcctctcaactgctgtgtcttttgtaaagctgatgaattgttcacagagtatgaatcaaagctattttccaacacagtcaaaggccagactgcaatatttgaggagatgatagaagatgaagaaggactggtggatgatcgagcacctaccaccacccggggtctctgggcaataagtgagacagagcgatctatgaaagcaatactatcatttgcaaaatcacataggtttgatgttgaatttgttgatgaaggaagcacttcaatggatctctttgaagcatggaagaaggaatataagttgcttcatgaatattggatggctctgaggaatcgtgtgtctgctgttgatgaacttgcaatggctacagaacgactaagagtgcgtgatcctagggagccaaagcctaatccgcctgttcttcatatcattgaaccacatgaggtagaacaaaaccgaataaaactactaaatgataaagctgttgctacatcacagcttcagaaaaaacttgggcagcttctttacctaactaatttggagaagtctcaagataaaacatcgggaggtgttaatccagaaccttgcccaatctgtgctcgacagctaggaaaacagtgggcggtactgacctgtggtcactgtttctgtaatgaatgcatttctataattattgaacaatacagcgtgggatctcacagaagctccattaagtgtgcaatctgccgccagaccacatctcacaaagaaatctcgtatgtctttacctcagagaaagcaaaccaggaggaggacatccctgtgaagggcagccattctacaaaagtggaagctgtggtcagaactctgatgaaaatacagcttagagatccaggggccaaagcactcgttttctcaacgtggcaagatgtattagatattatttcaaaagctcttactgacaacaacatggaatttgcacaaatcagtcgtgttaagacatttcaggagaacctttcagcatttaaacgtgatccccaaatcaatattttgctgctgcccctgcacacaggttctaatggattaactatcattgaagcaactcatgttctcttggtggagcccatattgaaccctgcccatgagcttcaggccatagggagggtgcaccgaattggacagacaaaacctactattgtacacagattcttaattaaagcaacaatagaagaaagaatgcaggcaatgctgaaaactgctgagagaagtcacacgaactcatcagcaaagcattcagaggcctctgtcttgactgtggctgacctggcagacctatttaccaaagaaactgaagagcttgaatgaactacacttgattcattccatggactttagtgtattaataaactttcatagctgtagagcaaagttacaagttttaaaaacccagtagataacagtaacactgttcctagttgaatgaatttgtttattgttctggttatacagtggcttagtacttattgagtccttttcaatatactgtttccaaaagatctataaagattcttaattttacactcctaataaaacatttattttttgtcccaaatagtatctatatctgatgatgcaagggaattaagagaggtaatgtgtacttttcttatgataagatcactcataaaacagttggaataagaaagtaaactagtctagttttatatgacctggtatattgaacaaacatagatctcagttttggaagggccttcagtatctttagcacatgattcaccaatatcttgttagtaatattttaagtagatggtgaattatattaacccattttttgagtaggcttataccataactttacacctagtaattttgcagtaatacaactccttacaataagttaatgttagacagttatttgtttcttctgcatcttgctactgaaaacaaaaacaatgaaaatattcatatttctattatgtgtaaagtggtgttagtgtaaacagttaactccaaatagattttttaatgtattaatgattaccttaaaattatctgctttccctactcttaatttttcttattgttaaagtttttatatgttgtaagattgatgtatctgtaaatattgcagtttttatgattttgttttattataacatggaaatatggcagggttttttttttaatcattattatactgatgtcttggattctttttctttcccatgttcttgtgtaactagactttttagccagtgatatctgaattcatggtagagttgttatttcactccagttgttcctcccttaagcacgagtctttgaccttttgaacaagagaatgccatgttcacataagcaaatcgttgcaagtgataataacgaagtgtttcttcttctgtgaggaattaatggactttgaaattgctgaataaggagtagcttgttacatcctagccatttgatgtaatacaaatgttcttatttggcttccttttctttgagccttcagagttttaaaggaccctctttgaccctcaggacttattagctcccagttagctgcctggaaaactcatagaaccattccctagatcccatttgtccgagaggctttggtgcttagttaaggtgcattttcttactctaggtttatagagtgatgtaattcaactgaagaaaacacacacatgcttttggtgcttattcttcagttaaaaggaggtatccattgattcttttatatgaaattaaatcaaactgttatttttatagctcttcacatattaaaaagagctttcatgtacatttacgtgtttaatttacccaacaatcttgtgaagttgatataattaatatctttactgttttaaaggtagagaactgaagttcagatatattgaaactttcttaggacatgtagcaagtaaacagtggagcccaggtcccctgactccatatcttacattcttttactaataacatgtcatgctgagattcagaaggcccatgcttataatttaaaaatgagactatttcagtatttctttgtgtttgttttctactattcctttctgagctatgttttagagaaaacaatttgatatgcttcaattttgtaaaatcaaattacatatatataaaaaaacctatctgtattagacaaaaactgtttttatttatttctgtaagatatccattaaataaatattttagtggaaatgaaaaaaaaaaaaaa

SEQ ID NO: 28 - protein, 1683 aa, Homo sapiens
MSSRRKRAPPVRVDEEKRQQLHWNMHEDRRNEPIIISDDDEQPCPGSDTSSAHYIILSDSLKEEVAHRDKKRCSKVVSFSKPIEKEETVGIFSPLSVKLNIVISPYHFDNSWKAFLGELTLQLLPAQSLIENFSERSITLMSSESSNQFLIYVHSKGEDVEKQKKEPMSICDKGILVESSFSGEMLEDLGWLQKKRRIKLYQKPEGNHIIKVGIYLLEAGLAKLDFLSDANSRMKKFNQLMKKVMEKLHNSIIPDVLEEDEDDPESEPEGQDIDELYHFVKQTHQQETQSIQVDVQHPALIPVLRPYQREAVNWMLQQECFRSSPATESALHFLWREIVTSEGLKLYYNPYTGCIIREYPNSGPQLLGGILADEMGLGKTVEVLALILTHTRQDVKQDALTLPEGKVVNYFIPSHYFGGKLKKTEIQNIEFEPKEKVQCPPTRVMILTAVKEMNGKKGVSILSIYKYVSSIYRYDVQRNRSLLKRMLKCLIFEGLVKQIKGHGFSGTFTLGKNYKEEDICDKTKKQAVGSPRKIQKETRKSGNKDTDSEYLPSDTSDDDDDPYYYYYKSRRNRSKLRKKLVPSTKKGKSQPFINPDSQGHCPATSDSGITDVAMSKSTCISEFNQEHETEDCAESLNHADSDVPPSNTMSPFNTSDYRFECICGELDQIDRKPRVQCLKCHLWQHAKCVNYDEKNLKIKPFYCPHCLVAMEPVSTRATLIISPSSICHQWVDEINRHVRSSSLRVLVYQGVKKDGFLQPHFLAEQDIVIITYDVLRSELNYVDIPHSNSEDGRRLRNQKRYMAIPSPLVAVEWWRICLDEAQMVECPTVKAAEMAQRLSGINRWCISGTPVQRGLEDLFGLVVFLGIEPYCVKHWWVRLLYRPYCKKNPQHLYSFIAKILWRSAKKDVIDQIQIPPQTEEIHWLHFSPVERHFYHRQHEVCCQDVVVKLRKISDWALKLSSLDRRTVTSILYPLLRLRQACCHPQAVRGEFLPLQKSTMTMEELLTSLQKKCGTECEEAHRQLVCALNGLAGIHIIKGEYALAAELYREVLRSSEEHKGKLKTDSLQRLHATHNLMELLIARHPGIPPTLRDGRLEEEAKQLREHYMSKCNTEVAEAQQALYPVQQTIHELQRKIHSNSPWWLNVIHRAIEFTIDEELVQRVRNEITSNYKQQTGKLSMSEKFCDCRGLQFLLTTQMEELNKCQKLVREAVKNLEGPPSRNVIESATVCHLRPARLPLNCCVFCKADELFTEYESKLFSNTVKGQTAIFEEMIEDEEGLVDDRAPTTTRGLWAISETERSMKAILSFAKSHRFDVEFVDEGSTSMDLFEAWKKEYKLLHEYWMALRNRVSAVDELAMATERLRVRDPREPKPNPPVLHIIEPHEVEQNRIKLLNDKAVATSQLQKKLGQLLYLTNLEKSQDKTSGGVNPEPCPICARQLGKQWAVLTCGHCFCNECISIIIEQYSVGSHRSSIKCAICRQTTSHKEISYVFTSEKANQEEDIPVKGSHSTKVEAVVRTLMKIQLRDPGAKALVFSTWQDVLDIISKALTDNNMEFAQISRVKTFQENLSAFKRDPQINILLLPLHTGSNGLTIIEATHVLLVEPILNPAHELQAIGRVHRIGQTKPTIVHRFLIKATIEERMQAMLKTAERSHTNSSAKHSEASVLTVADLADLFTKETEELE

SEQ ID NO: 29 - protein, 1683 aa, Homo sapiens
MSSRRKRAPPVRVDEEKRQQLHWNMHEDRRNEPIIISDDDEQPCPGSDTSSAHYIILSDSLKEEVAHRDKKRCSKVVSFSKPIEKEETVGIFSPLSVKLNIVISPYHFDNSWKAFLGELTLQLLPAQSLIENFSERSITLMSSESSNQFLIYVHSKGEDVEKQKKEPMSICDKGILVESSFSGEMLEDLGWLQKKRRIKLYQKPEGNHIIKVGIYLLEAGLAKLDFLSDANSRMKKFNQLMKKVMEKLHNSIIPDVLEEDEDDPESEPEGQDIDELYHFVKQTHQQETQSIQVDVQHPALIPVLRPYQREAVNWMLQQECFRSSPATESALHFLWREIVTSEGLKLYYNPYTGCIIREYPNSGPQLLGGILADEMGLGKTVEVLALILTHTRQDVKQDALTLPEGKVVNYFIPSHYFGGKLKKTEIQNIEFEPKEKVQCPPTRVMILTAVKEMNGKKGVSILSIYKYVSSIYRYDVQRNRSLLKRMLKCLIFEGLVKQIKGHGFSGTFTLGKNYKEEDICDKTKKQAVGSPRKIQKETRKSGNKDTDSEYLPSDTSDDDDDPYYYYYKSRRNRSKLRKKLVPSTKKGKSQPFINPDSQGHCPATSDSGITDVAMSKSTCISEFNQEHETEDCAESLNHADSDVPPSNTMSPFNTSDYRFECICGELDQIDRKPRVQCLKCHLWQHAKCVNYDEKNLKIKPFYCPHCLVAMEPVSTRATLIISPSSICHQWVDEINRHVRSSSLRVLVYQGVKKDGFLQPHFLAEQDIVIITYDVLRSELNYVDIPHSNSEDGRRLRNQKRYMAIPSPLVAVEWWRICLDEAQMVECPTVKAAEMAQRLSGINRWCISGTPVQRGLEDLFGLVVFLGIEPYCVKHWWVRLLYRPYCKKNPQHLYSFIAKILWRSAKKDVIDQIQIPPQTEEIHWLHFSPVERHFYHRQHEVCCQDVVVKLRKISDWALKLSSLDRRTVTSILYPLLRLRQACCHPQAVRGEFLPLQKSTMTMEELLTSLQKKCGTECEEAHRQLVCALNGLAGIHIIKGEYALAAELYREVLRSSEEHKGKLKTDSLQRLHATHNLMELLIARHPGIPPTLRDGRLEEEAKQLREHYMSKCNTEVAEAQQALYPVQQTIHELQRKIHSNSPWWLNVIHRAIEFTIDEELVQRVRNEITSNYKQQTGKLSMSEKFRDCRGLQFLLTTQMEELNKCQKLVREAVKNLEGPPSRNVIESATVCHLRPARLPLNCCVFCKADELFTEYESKLFSNTVKGQTAIFEEMIEDEEGLVDDRAPTTTRGLWAISETERSMKAILSFAKSHRFDVEFVDEGSTSMDLFEAWKKEYKLLHEYWMALRNRVSAVDELAMATERLRVRDPREPKPNPPVLHIIEPHEVEQNRIKLLNDKAVATSQLQKKLGQLLYLTNLEKSQDKTSGGVNPEPCPICARQLGKQWAVLTCGHCFCNECISIIIEQYSVGSHRSSIKCAICRQTTSHKEISYVFTSEKANQEEDIPVKGSHSTKVEAVVRTLMKIQLRDPGAKALVFSTWQDVLDIISKALTDNNMEFAQISRVKTFQENLSAFKRDPQINILLLPLHTGSNGLTIIEATHVLLVEPILNPAHELQAIGRVHRIGQTKPTIVHRFLIKATIEERMQAMLKTAERSHTNSSAKHSEASVLTVADLADLFTKETEELE

SEQ ID NO: 30 - protein, 1683 aa, Homo sapiens
MSSRRKRAPPVRVDEEKRQQLHWNMHEDRRNEPIIISDDDEQPCPGSDTSSAHYIILSDSLKEEVAHRDKKRCSKVVSFSKPIEKEETVGIFSPLSVKLNIVISPYHFDNSWKAFLGELTLQLLPAQSLIENFSERSITLMSSESSNQFLIYVHSKGEDVEKQKKEPMSICDKGILVESSFSGEMLEDLGWLQKKRRIKLYQKPEGNHIIKVGIYLLEAGLAKLDFLSDANSRMKKFNQLMKKVMEKLHNSIIPDVLEEDEDDPESEPEGQDIDELYHFVKQTHQQETQSIQVDVQHPALIPVLRPYQREAVNWMLQQECFRSSPATESALHFLWREIVTSEGLKLYYNPYTGCIIREYPNSGPQLLGGILADEMGLGKTVEVLALILTHTRQDVKQDALTLPEGKVVNYFIPSHYFGGKLKKTEIQNIEFEPKEKVQCPPTRVMILTAVKEMNGKKGVSILSIYKYVSSIYRYDVQRNRSLLKRMLKCLIFEGLVKQIKGHGFSGTFTLGKNYKEEDICDKTKKQAVGSPRKIQKETRKSGNKDTDSEYLPSDTSDDDDDPYYYYYKSRRNRSKLRKKLVPSTKKGKSQPFINPDSQGHCPATSDSGITDVAMSKSTCISEFNQEHETEDCAESLNHADSDVPPSNTMSPFNTSDYRFECICGELDQIDRKPRVQCLKCHLWQHAKCVNYDEKNLKIKPFYCPHCLVAMEPVSTRATLIISPSSICHQWVDEINRHVRSSSLRVLVYQGVKKDGFLQPHFLAEQDIVIITYDVLRSELNYVDIPHSNSEDGRRLRNQKRYMAIPSPLVAVEWWRICLDEAQMVECPTVKAAEMAQRLSGINRWCISGTPVQRGLEDLFGLVVFLGIEPYCVKHWWVRLLYRPYCKKNPQHLYSFIAKILWRSAKKDVIDQIQIPPQTEEIHWLHFSPVERHFYHRQHEVCCQDVVVKLRKISDWALKLSSLDRRTVTSILYPLLRLRQACCHPQAVRGEFLPLQKSTMTMEELLTSLQKKCGTECEEAHRQLVCALNGLAGIHIIKGEYALAAELYREVLRSSEEHKGKLKTDSLQRLHATHNLMELLIARHPGIPPTLRDGRLEEEAKQLREHYMSKCNTEVAEAQQALYPVQQTIHELQRKIHSNSPWWLNVIHRAIEFTIDEELVQRVRNEITSNYKQQTGKLSMSEKFHDCRGLQFLLTTQMEELNKCQKLVREAVKNLEGPPSRNVIESATVCHLRPARLPLNCCVFCKADELFTEYESKLFSNTVKGQTAIFEEMIEDEEGLVDDRAPTTTRGLWAISETERSMKAILSFAKSHRFDVEFVDEGSTSMDLFEAWKKEYKLLHEYWMALRNRVSAVDELAMATERLRVRDPREPKPNPPVLHIIEPHEVEQNRIKLLNDKAVATSQLQKKLGQLLYLTNLEKSQDKTSGGVNPEPCPICARQLGKQWAVLTCGHCFCNECISIIIEQYSVGSHRSSIKCAICRQTTSHKEISYVFTSEKANQEEDIPVKGSHSTKVEAVVRTLMKIQLRDPGAKALVFSTWQDVLDIISKALTDNNMEFAQISRVKTFQENLSAFKRDPQINILLLPLHTGSNGLTIIEATHVLLVEPILNPAHELQAIGRVHRIGQTKPTIVHRFLIKATIEERMQAMLKTAERSHTNSSAKHSEASVLTVADLADLFTKETEELE